SEQUENCE LISTING (74 sequences)

SEQ ID NO: 1 - protein, 254 aa, Homo sapiens
MDPAASSCMRSLQPPAPVWGCLRNPHSEGNGASGLPHYPPTPFSFHQKPDFLATATAAYPDFSASCLAATPHSLPQEEHIFTEQHPAFPQSPNWHFPVSDARRRPNSGPAGGSKEMGTSSLGLVDTTGGPGDDYGVLGSTANETEKKSSRRRKESSDNQENRGKPEGSSKARKERTAFTKEQLRELEAEFAHHNYLTRLRRYEIAVNLDLSERQVKVWFQNRRMKWKRVKGGQPISPNGQDPEDGDSTASPSSE

SEQ ID NO: 2 - DNA, 2674 bp, Homo sapiens
agccctctgcaggcatttgctctgggctccaggacgaactcctcgtcagctggtgcgtctgggttgggacagtgaaaatgtttagtgtcattggcgacaaatacacatacaggggatcccagtaggtaggctggtgcattggaggggcaggagcaagcccgacataggtgtgtgcacacacataggagtagctggccacatgtgtgtgtactttaaggagagactttagttttgggtttttttttttttttggttcctggggtaaattttctgttgaacatttttccctcctatttagttttttttcttttgcatttttaaaaatttgaacataaaaagtataaagaatcaaatctttgaaaggaccgaggcgtgcagcggacagcagatggatcccgcggccagcagctgcatgaggagcctccagcccccagcccctgtctggggctgccttcgaaacccccactcggaaggcaatggggcctcagggctaccccactacccgcccaccccgttctccttccaccagaaaccagacttcctggcgacagcgacggcagcgtaccctgacttctcagcctcctgcctggcagccaccccacacagcctgccccaggaggagcacatcttcactgagcagcaccccgctttcccacagtcccccaactggcacttccctgtctcagacgcccggcgcaggcccaactcaggcccggcagggggttccaaggaaatggggaccagcagcctgggcctggtggacaccacaggaggcccaggcgatgactacggggtgcttgggagcactgccaatgagacagagaagaaatcatccaggcggagaaaggagagttcagacaaccaggagaacagagggaagccggagggcagcagcaaagcccgcaaggagaggacggccttcaccaaggagcagctgcgagagctggaggcagagtttgcccatcataactacctgactcggctccgcagatatgagattgcggtaaacctggacctctctgagcgccaggtcaaagtgtggttccagaaccgaaggatgaagtggaagcgtgtgaagggaggtcagcccatctcccccaatgggcaggaccctgaggatggggactccacagcctctccaagttcagagtgagattctgcatggaggaaaaatgactaaggactgagccccctacccaactacccccaccccaatcccaccttcaccctcttccttccccagccagggcagcctctccacatctttccctgactcttggatatgaaactgcccagcattcctgggagtcttaggattttctaggaagttctgtccagcctcttagcagcctcttccctagggcctttgctcccacactctcatggaatcagacagagatcctaccgggccggatgaatctggaaacagcttcagagatactgcttctcagcgtctcttggctgccacccatgcctcctcctaccgctgttctcctaggtcagccaggcctcctcctggtctggacaccacctggcctggtgggagaggagctttggaaccagctggcgactcggaaagtaaatgcttcaaaaggaaggaaatgacagagacacacgcccttgcccaccttcctctgtaggctgcacatctgaggctttggggccccttagttgtcccgaaaccccaagaaaaatcagaatgaggagagtcaaggacagcaactcagctgctgcaagccagaaacacatccctgtctccaaatttgttggctaagtggagacacttctgagaactgactagagaagacagaaaaatagcccgatgtaggtttcggtgtccccatataggcccgtccacacaggcttgactgggtggacaagaatgaacccatgacagcacctgctgcttcaaaatcaaaatcaatttagggatacagcaggggctgttgggctgtgctccagagaaaaggagcagctactccttttaaatccacgatttctggattgaaaacctgtccagatgctgagttgttgggctgaacaactaggagctgaaaacaacgtagaggctggaaagtgtcccctgcattctggaggggaggggagataataaggagggctgctgggtgagggcctggagatgtggaaccctggagtggaaggttctccagtgacagtgtcctgtgactgcaaaaggggacaagaaaatccctcttcctccatgggatggatttaagctcttgctgtgtgttctacaaatgctgttattgtgggaggaaatgctaggtttttgtgtgtggactgcccagacctcagccaggtcttctggagatgacatttgaggactgatggccaaagagcatgggggactgaagccctggctgcctcagcgctctgtctcccaacaccagctggtgttgcagagggaggtcaacgtgagtttggatctcttgtacgcagatgtaatcattcacatgtaaaaataaccccacctccccaccccaaaaagggcaagagctgtggaaaatgattgccaaatgagatggctggttagagcatgattttttctaaagcatacttcatatattttcttaagattacatcaagctaattgtgcgagctcaattcactttgtaagaaaactctcggagaaataaaatcaataaaaagcaaa

SEQ ID NO: 3 - DNA, 5883 bp, Homo sapiens
cttgcaatcccagcactttgggaggctgaggcgggtggatcagctgaggtcaggagtttgagaccagcctgcccaacatggtgaaaacccatctctactaaaaatataaaaactaaaggcgggcgtggtggttcgtgcctgtaagcccagcactttgggaggctgaggcaggaggatcacaaggtcagaagatcgagaccatcctggctaacatggtgaaaccccgtctctactaaaaatacaaaaaattagcctggtgtggtggcgggcgcctgtagtcccagctactcaggaggctgaggcaggagaatggcgtgaacccaggaggcggagcttgcagtgagccgagatcgcgccattgcactccagcctgggtgagagtgtgagactctgtctcaaaaaaaaaaaaaaaccatatatatatatatagtttatatatatggtttatatatatagtttatagtttatatatatggtttatatatatagtttttatatatatagtttatatatacatatatatataaactagccgggggtggtggtggatgcctgtaatcccagctactcgggaggctgaggcaggagaattgcttgaacccaggagacggaggttgcagtgagccaacacggtgccactggactctagcctgggtgacagagtgagactctgtctcaaaaaaaaagaataattttttctttaacagaacaaattgcctccctgagtcggaccagctgcccgtggatcctacgctgcagtcacaccagactcctcatgttgtggggtaagtgtgtcccctttctttcctgcttctgtctttatacacacagtccctttcctttgatacgttttctcctctcgtctgcctctgagccagccccaagagtcttctccgtgcttcctgactcctctctgcttagctgttaccccatttgttcctctgcaccatagttaaacctatttacaggtctgtgtcccccaccaaactgtgagctccttgaaggcaggcgatgtccattttgtatccccccgaaaaccatccccctacccccagtacagactctggcacaaaataggcattaagtaaatgaatgaaggaatgaatggatggaaaggatgccattgtaggggggaaagagtaatatctattacttttatttaaaaaaaacttttaatttccacttctaggcatatatcttttccttatctgtcacaaagttcatggcttgagatccctataacaaaagatagattaacaagagaaaagcatacacatttatataataagtttccgtgacatgggagatgaagacccagggaaatagggaatactgtgtatattttatgcttaggtttaatgaggagtagagcgttgtgtagaaacatgatggacgaggccaggcgtggtggctcacgcctgtaatcccagcactttgggaggccgaggcgggtggatcacttgaggtcaggagttcaacaccagcctggccaacaggatgaaaccccgtctctactaaaaatacaaacaattagctgggcatggtagcgcacgcctgtaatcccagctactcgggaggctgagacaggagaattgctggaacccaggaggtagaggttgcagtgagcggagaacacactactgcactccagcctgggagacagagcgagactccatctcaaaaaaaaaaaagaagaagaaatatgatggaagaaagggggtatgatctaatagtaatcaacttgggggagcttagcaaggcctgtttcttcagattcttctctgcttcttcattcctttcctctggatatagggaggacccctctggaatgagggtcttatgacctactttagaagaaagacagaaaattctttcatgatctgcttcaggggagaatagtagcagcaagtcagagagatcttcctccttctgctgcttttgccgaggtgctgtattttggggtagcatgtcctgaactccatcaccatcttgtttgcccctttaggatctgccagcaagattttgagcaaaatcctaatctctggcctcattttagagcttgtagcatcaggtttggaggctaggggttcctggtgccccagcaccagagaggagggtgagaaggccaccttgggagagctgggccatttcaggggaggaaattaaaactaaattgcattaattggttttcatattgaaccctgcttcttaagtgtgctaaatgtctggttaaacaaatagctaaaaagatagaatcactttgctatcattttttgtggtaagggaaaagacaaaaaaacaaagatgttgatttaagatatagcctagggctggggggctgggggaaacctggtgaaggctcctgttgtttggggtcggaggtcgcaggagcagcggctgcctggggatcaggctcaaggttctggaatgatggaaaaacctggcctgacgcttttcctcagcggacccttcactggctaacaggagccagctgagcgaacacagaggcgctgtaaccggcgcagatcccagcttcctctgaaatttccaggcctcttttttcctgtcggattccagacagatgaaattttcctggccccggcccattcctggcagcatctcccctctgaatcatcataaatcagggcttggcggggagcggtgggattttcctgattttcctgatcgacacgctgctgccctggtaaaatggggctccgtttccaaggctaaaaatagctctggggtgcttgcctgagttctccccatcgcaagttgctgcttcgtattgtaaatattcatatctgtctatgattattatttcatcgagaagggctgtggacgtgcagatgcgggccgctgggggcctcggtggtctgaagtgacaccattgttcacaggatcatatgcgggggcctgtgcattcctcaaagcctccatcatccaaggagcccccaattaatttcaatacaaacaccggatatggcctcccccgcgcctttgcagccataacacatgaggtcatgctgctttggttaacccgaggagttgcagagtgagatggggaggcttctaggccccaggggaggcctggggtatctgccctgcctggtttaggcgaggctgtttactgcaatggggtctacggcacacccacactctctcgccttccttcactcagaaactgaaatctctgattcccgatgtctcttcccagccactctccccatctctagccacttggctgccaacccaccacccacccctgtggttctttgcaaactccttaccctccgtgctcttgcatacacagcttcattcacctggggcatctttttacctcttcatcaggctccttctggaagattcacctcaggagtttcttccttcaggaagcatttcctgatcactccattcagggttaagtactgttttcttggctgggagcagtggctcatgcctgtagtcttggtactttggaagacggaggcaggcggatcacttgagctcaggagttcgagaccagtctggccaacatggtgaaaccacatctttactaaaaatacaaaaattagctgggcacagtggtgcatgcctgtaatcccagctactcgggaggccgaggcaggagaatcgcttgaacacagaaggtggaggttgcagtgagccgagattgcgccaatgcattccagcctgggtgacagagtgagactccatctcaacaacaacaacaaaattaaatttaaaaaaaaagtacttgctgggcgtggtggctcgcgcctgtaatcccagcgcttggggaggccgaggctggtggatcacctaaggtcaggagttcaagaccagcctggccaacatggtgaaatcccatctctactaaaaatacaaaaaagtagctgggcatggtggcaggcgcctgtgatcccagctactcaggaggctgagtcaggagaactgcttgaacccgggaggcagaggttgcagtatgccgagatcatgccattgcactccagcctgggcaacaagagcaaaattccatctcaaaaaaaaaaaactaccactttcagccgggcagggtggctcacgcctataatcccagcactttgggaggccgaggtaggtggatcacaaggtcaggagatcgagaccatcctggctaacacaatgaaaccccatctctactaaaaatacaaaaaaattagccaggcgtggtggtgggtgcctgtagtcctagctactcaggaggctgaggcaggagaatggcgtgaacctgggaggcggagcttgcagtgagccgagatcactgcaactgcactccagcctgggcaacagagggagactctgtctcaaaaaaaaaattccactttctcttctgtgttcctacagcttattgattctcaaacattagccctcatcagaatcgcatggagggtttgctaaaacaccgattgctgggcccctcccagtttcagaatcaataggtctgaagtagggcttgcacatttgcatttctaacaagatctcaggagacactgatgctgctacagccccctgtgatcacttcctccattggtggaacttaccctgctgtgctgcaattacccgcctttttcttttcttttcttttttttttttttgtttgagacagggtctcactctgtcacccaggctggagtgcagtggcgccatcttggctcactgcaacctctgcccctgggtttaaacaattctcctgcctcagcctcccgagtagttgggattacaggtgcctgtcaccacacccggttaatttttgtatttttagtagagacggggtttcaccatgttggccaggctggtctcaaactcctgacctcaagtgatccacccgcctcgacctcccaaactgctgggattacaggcgtgagccacctgtccggccacctgcttattttttgttccctcccactgggagggctgggactgtcttttccatttctctatcctactgcttggcaaacagtgaagctgatcactggaggtttgttgactgaatgaattgtggatttggaaccaacctgctagttgtagagctcagttgaggggaggaggtctgctggtgagagggctggttctcagggcttttggggtcatgagtatgttacctgaaagggggcccaatccagatcccaagagaggattcttggaccttgcagaagaaagaattcggggcgagtttatagagtaaagtgaaagcaagtttattaagaaagtaaacgggctgggcgtggtggctcactcctgtaattccagcactttgggaggccgaggaggcggatcaccttaggtcaggagttcgagaccagcctgatcaatatggagagaccccatctctactaaaaatacaaaattagccgggcgtggtgactcacgcctgtaatcccagctactcaggagactgaggcaggagaatcgctggaacccaggaggtggaggtggcagtgagccaagatcgcgccattgcactccaacctgagcaacaaaagcaaaactccgtctcaaaaaaaaaaaaaagaaactaaatgaataaagaatggatactgcataggcagagcggcggcatgagttgcttgactgagtatgcttattgttccggttttttttttttttttttgagacggagtctcgctctgttgtccaagctggagtgcagtggtgcaattcagctcactgcaacctccgcctcctggtttctagcaattctcctgcctcagcctcccaagtagttgggattacagctgtgcgccacaacatcaggctaattttttatatttttagtagagacaggttttcatcatgttggccaggctggtcttgaactcctgacctcaagtgatcctcctg

SEQ ID NO: 4 - protein, 1368 aa, Streptococcus pyogenes [sequence omitted]

SEQ ID NO: 5-14 - skipped (10 entries)

SEQ ID NO: 15 - DNA, 4101 bp, Streptococcus pyogenes
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 16 - protein, 1307 aa, Francisella novicida
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 17 - DNA, 3921 bp, Francisella novicida
atgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatgagaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggcctctggctgggacgtgaataaggagaagaacaatggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgatcggggcgagagaaacctgatctatatcacagtgatcgcctccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgcgaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaac

SEQ ID NO: 18 - DNA, 20 bp, Artificial Sequence
  A synthetic oligonucleotide
aggcttcacttaccctagac

SEQ ID NO: 19 - DNA, 20 bp, Artificial Sequence
  A synthetic oligonucleotide
caataatgggctctgtaagg

SEQ ID NO: 20 - DNA, 26 bp, Artificial Sequence
  A synthetic oligonucleotide
gttcagagttctacagtccgacgatc

SEQ ID NO: 21 - DNA, 21 bp, Artificial Sequence
  A synthetic oligonucleotide
agacgtgtgctcttccgatct

SEQ ID NO: 22 - DNA, 20 bp, Artificial Sequence
  A synthetic oligonucleotide
attccaagcctgaaagagca

SEQ ID NO: 23 - DNA, 20 bp, Artificial Sequence
  A synthetic oligonucleotide
agacactggcttggcttagg

SEQ ID NO: 24 - DNA, 20 bp, Artificial Sequence
  A synthetic oligonucleotide
cagatgagccacaagaggtg

SEQ ID NO: 25 - DNA, 20 bp, Artificial Sequence
  A synthetic oligonucleotide
ccaggcagatgacagtcaga

SEQ ID NO: 26 - DNA, 20 bp, Artificial Sequence
  A synthetic oligonucleotide
atggttcccttcaaccactg

SEQ ID NO: 27 - DNA, 20 bp, Artificial Sequence
  A synthetic oligonucleotide
aagcgttgccctcagtatgt

SEQ ID NO: 28 - DNA, 20 bp, Artificial Sequence
  A synthetic oligonucleotide
gcttggatcagctccctaca

SEQ ID NO: 29 - DNA, 20 bp, Artificial Sequence
  A synthetic oligonucleotide
tgggtcaggttcaagactcc

SEQ ID NO: 30 - DNA, 20 bp, Artificial Sequence
  A synthetic oligonucleotide
gctggggtacaggcatacac

SEQ ID NO: 31 - DNA, 20 bp, Artificial Sequence
  A synthetic oligonucleotide
ggccacaagacactccaagt

SEQ ID NO: 32 - DNA, 20 bp, Artificial Sequence
  A synthetic oligonucleotide
gggagagtagtgcggaacag

SEQ ID NO: 33 - DNA, 20 bp, Artificial Sequence
  A synthetic oligonucleotide
actcatggggagctgctgta

SEQ ID NO: 34 - DNA, 20 bp, Artificial Sequence
  A synthetic oligonucleotide
ctgcgctgcactttagaagt

SEQ ID NO: 35 - DNA, 19 bp, Artificial Sequence
  A synthetic oligonucleotide
ttggaatttgagccaatgg

SEQ ID NO: 36 - DNA, 20 bp, Artificial Sequence
  A synthetic oligonucleotide
ttggtcatcgggagactccg

SEQ ID NO: 37 - DNA, 20 bp, Artificial Sequence
  A synthetic oligonucleotide
aacgacaatagcctttcccc

SEQ ID NO: 38 - DNA, 20 bp, Artificial Sequence
  A synthetic oligonucleotide
tacttttggatacacccacc

SEQ ID NO: 39 - DNA, 20 bp, Artificial Sequence
  A synthetic oligonucleotide
agggcaacgcttgttaagat

SEQ ID NO: 40 - DNA, 20 bp, Artificial Sequence
  A synthetic oligonucleotide
ctaaagtttgaaatccaacg

SEQ ID NO: 41 - DNA, 20 bp, Artificial Sequence
  A synthetic oligonucleotide
cacaggatatggagtccgtg

SEQ ID NO: 42 - DNA, 20 bp, Artificial Sequence
  A synthetic oligonucleotide
gagcgtagccaaaattctgt

SEQ ID NO: 43 - DNA, 20 bp, Artificial Sequence
  A synthetic oligonucleotide
accctactaggacatggcaa

SEQ ID NO: 44 - DNA, 20 bp, Artificial Sequence
  A synthetic oligonucleotide
agcttgcgagaaattcacgt

SEQ ID NO: 45 - DNA, 20 bp, Artificial Sequence
  A synthetic oligonucleotide
tgcctagcttagtagaaggc

SEQ ID NO: 46 - DNA, 20 bp, Artificial Sequence
  A synthetic oligonucleotide
catacgcagctctgtccact

SEQ ID NO: 47 - DNA, 20 bp, Artificial Sequence
  A synthetic oligonucleotide
gtcattatcacgcctccccg

SEQ ID NO: 48 - DNA, 20 bp, Artificial Sequence
  A synthetic oligonucleotide
tgaaccaagactccgacggt

SEQ ID NO: 49 - DNA, 20 bp, Artificial Sequence
  A synthetic oligonucleotide
cgtggtttgtaacctctgaa

SEQ ID NO: 50 - DNA, 20 bp, Artificial Sequence
  A synthetic oligonucleotide
cagatgagccacaagaggtg

SEQ ID NO: 51 - DNA, 20 bp, Artificial Sequence
  A synthetic oligonucleotide
ccaggcagatgacagtcaga

SEQ ID NO: 52 - DNA, 20 bp, Artificial Sequence
  A synthetic oligonucleotide
attaagctgccgagctcttg

SEQ ID NO: 53 - DNA, 22 bp, Artificial Sequence
  A synthetic oligonucleotide
ttgtggtttggtatgagacagc

SEQ ID NO: 54 - DNA, 20 bp, Artificial Sequence
  A synthetic oligonucleotide
gagcacaggccagatctctt

SEQ ID NO: 55 - DNA, 20 bp, Artificial Sequence
  A synthetic oligonucleotide
aacagcagcagcagagcata

SEQ ID NO: 56 - DNA, 20 bp, Artificial Sequence
  A synthetic oligonucleotide
tagggacagctgggattgtc

SEQ ID NO: 57 - DNA, 20 bp, Artificial Sequence
  A synthetic oligonucleotide
gcctcctccagccttctact

SEQ ID NO: 58 - DNA, 20 bp, Artificial Sequence
  A synthetic oligonucleotide
tgagcgaacacaaacagagg

SEQ ID NO: 59 - DNA, 20 bp, Artificial Sequence
  A synthetic oligonucleotide
tggaatccgacaggaaaaag

SEQ ID NO: 60 - DNA, 20 bp, Artificial Sequence
  A synthetic oligonucleotide
agtttgccccagaccctact

SEQ ID NO: 61 - DNA, 20 bp, Artificial Sequence
  A synthetic oligonucleotide
accttgagccaggaccctat

SEQ ID NO: 62 - DNA, 20 bp, Artificial Sequence
  A synthetic oligonucleotide
gcagtggacagcagatggat

SEQ ID NO: 63 - DNA, 20 bp, Artificial Sequence
  A synthetic oligonucleotide
tgcctcaaattccacaaaca

SEQ ID NO: 64 - DNA, 20 bp, Artificial Sequence
  A synthetic oligonucleotide
ggctgagagaggagggtctt

SEQ ID NO: 65 - DNA, 20 bp, Artificial Sequence
  A synthetic oligonucleotide
caggaggaggagggtattga

SEQ ID NO: 66 - DNA, 20 bp, Artificial Sequence
  A synthetic oligonucleotide
tgtgcaaaggacctgggttt

SEQ ID NO: 67 - DNA, 20 bp, Artificial Sequence
  A synthetic oligonucleotide
cttggaggacatggcaggtt

SEQ ID NO: 68 - DNA, 22 bp, Artificial Sequence
  A synthetic oligonucleotide
aaggaactcacctctggtttag

SEQ ID NO: 69 - DNA, 19 bp, Artificial Sequence
  A synthetic oligonucleotide
cctttgcctccctggaatt

SEQ ID NO: 70 - DNA, 24 bp, Artificial Sequence
  A synthetic oligonucleotide
ttaacactgggtggtggtgatggt

SEQ ID NO: 71 - DNA, 21 bp, Artificial Sequence
  A synthetic oligonucleotide
aggctttttgaacagctttgt

SEQ ID NO: 72 - DNA, 18 bp, Artificial Sequence
  A synthetic oligonucleotide
cacagactcgctggacag

SEQ ID NO: 73 - DNA, 31 bp, Artificial Sequence
  A synthetic oligonucleotide
cttcagaagcccaaaatataagctacaccga

SEQ ID NO: 74 - DNA, 4668 bp, Mus musculus
gcattaattttgagtttccaaaattcacattagatagtgtatagcttgtattctgagcttgaggcaagacaccctttccactttgtctggagcgagtggctcactggtatcttagagtctttgctcagtgtatgagaaacactgagtcccagaatgagcacctgcaagcccttctattcatctattagcccagggtgaccttgagcttgtgatcctgttgcctcaacacctgaatgctggttttatgggacacaccaccacacctaagtccaggactgggacactgaatgtcttctaagccttcttctcttctagctaccagcccctcccctgccggtgttgcagaatatctgataaaagttaacacccttgcctggctcagcagctcccactctctctctcctgattgatcccccacctccaaccctaatcattgcaccccaacttccacaatgtcacccagggcacctgcacatctcatctccctggcccttgactaaactttccagggtcctcagcacaaactccctgttgcctgaaattacatcagctttttgacgaaacttgagtcagatgtgtgtgtgtgtgtgtgaaataattctctcttttaaaaaatatccatttatttttatcttgtgtacatgaatgttttgtctgcatgcacgtctgtgtatcacgtgtgtgcagtacctgtagaggcctgaagagattgtgagccactatgtgggtgctgggaaccaaacctgggctgttaggaagaataaccacggagtcacctctccagcccagaaatgctattttctctctctcttttagaaaacagaacaaagagtctagcagagcaggcctagccctcagatcctctgccccagtgctagcagactccccatggtgtgggtgcatgtatatcccttctcttccctgcttctctcttcgctctgggtccattcctctacacccacccacaccccctgacttgcctcttttattttaaaacattagtttgtctatttgatgtttgtctgtaattgtgtgtgcttgaatgcagagctctgagggcaacttgtggagtcagatctctccgtctgtcatgtgggttctgagtttagaactcaggctattagctttggccgcaagccctttctcccactgagccattttagtagcccttacctgccttttgaagcagcgtcaatctcttctgtgcctggtgcctgatccaaggcccgcccccccaaacccccattttggttctttatttactgggctctatgaaccttgagggtaggtaacacctttcatatctcctctgtctctagtcccacaccctccacacagggtcaggcacaaagtaggcatgaatgagtgaatgagtgaatgaatgaatgaacgaaataaacactgcagcagaaaaaaatcttttccttattgtttcttggctagatctggggcagagctctgtgacccccctagcctcaaaccagagctcggaacatcaggtttggaggtccaggttcctggttcccctagcccaaagaggacaataaaggcctcctctggagagccgggtcactccaccggaggacattaaaacaaaactgaattaactactcttcacatggaacccagcttcgtgagtgtgctaagtgtcttattaaacaaacaaccctaaaagatagacgcgccccgctatcgtttctcatagtaagagaaaaaccaaaggcccagggctgggggctttgggggaaacctggctcttattctttggggccggaggtcactggaacagctgctgcctggggatcaagctcaaggttctggagagatggaaaaacctggcctgccgcttttcctccgcggcccctcagctgctaacaggagccagctgagcgaacacaaacagaggcgctgtgaccggcccagatcccagcttcctctgaaatttccagcctcctttttcctgtcggattccagacagatgaaactttcctggccctggcctgttcctggcagcatctctcctctgaatcaccataaatcaggtctgggggtgagcagtggttttttttcctgattgacaagctgctgcctcggtatagcaggatctcggcttcatcggctaataaaataggtcggggtggggtggggggggggggacgcgggagtgttgactaagccctccccgacacaagtctgtttaggtatccctttgtatttgaaagttttaaatctgtctatgattattatttcattgagaagggctgcggatatatagatatgggcctttggggggctgcggtggtcgtaagcaacaccattgtttgagggatcacataggaggcctgtacattccttaaagtctccatcatccatgagtccccaattaatttcaatacaaacaccggatctggcctcctctgtgtctctgtctctgcagccataacacatgaggtcatgcagttttggttaaccaaccggaggtgcctgatggaggtggggggcttccagccctcgggaggctggggtatctgccctgcctgactccagcagggccgtttattgaagcgaggggtcatgggcacccatactcacttctttgatggcttttcctcacccctgcccccttcatctcaagccacccatctgtcatagtgccaccctgtgtttttgttgtttgttttgtaacagggtcttggagtctcccaggctagtcaaggaaggccttgtacttctgatcttcctgtcttctgtccctggttttatgaaatgggggattgaatttagggcttcatgcatgtcaggcaagctacatgctacctcttttcccctttagttttaggtagggacagggtctcatgtatccaagaatgggaatggcttcagacttgctgtgtagctgaagatgatcttgaactcttgatcctgctgtgtccatctcttgcgtgctgggattataggcatgtaccacgctacacctggttggcactcctgggatcaaacccagtgctctgttcaatctaccctcccatccccccttcaccatcccctctccccctcccctccctcctttgcccctctctcccatccctcttcctaccttccccctctgtcccctccccttcctccccttccttccttccctctcctctccctcctcctgcctaccatcccctccctcccactccttaccatccctctccctaacaaacccttccctcacttttttctttttgacccctcccctaatcttcccttttaagataggatctcactgtgtagcctagcctggtctcaaattcaagaccctcctctctcagcctcctgggtgctggggtacaggcatacaccattacaggcttccaccctgtggttctttaaactctcctcccctttagtactctttgcatacgcagctctgtccacttggagtgtcttgtggccttttaatctgtctccttgtttacagcaggagccttcttcctccaggaaggctttctgcctcatttcatccggggttagttcctgcttctacacagcctgttggttctcagacagccattcacatcagaatcacctggagggtttatgaaagtgttgactggtagcctgctcccacattctgcattatctgacttggactgggctgggcctgccaacgtgaatttctcgcaagctctcaggggactccgatgctaccacagcctctggcgtcatcctccttgatatcatttatcctgctggcttgctactactcacttatttgtgtgcctctccctagggacagctgggattgtctttttcctctctaagcctatggtttggacagctgtgtgcacagtgaagctgctctcgctggaggtttgctgattaaatgagttgtagattcagaggaggctggctgactgcctagcttagtagaaggctggaggaggcgcctatctcccagaggaggctgttgggcaggagggctggttttcggggaccacaggatggtgagtggggattgtcttcatcaagggaaatgagcacaactctccccttctgtgcacatctctaggaagagatgggaggccagtggttcaggtgccaacaggaggaggagggtattgaactgtcttagttaaggttttactgctgtgaacagacaccatgaccaaggcaactcttataagggcaacatttaattagggctggcttacaggttcagaggttcagcccagtatcatcaaggtgggaacctggcagcatccagggcaggcatggtgcaggaagagctgagagttctacatctacatctgaaggctgctagcagaatactggcttccaggcagctaggatgagggccttaaagcccacacccacagtgacacacctactccaacagagccacaccttgtaataaagccactccctggactgagcatatacaaaccatcacatgaaatccacctgatttttctctctgagacctgggaaggctggatggtgaaagaatacatggctgttcttttcgttccatggtgtgtgtgtgtatgtacatgcacatgtgtgtgtgcatatgtgtaggccagaagtcaatggtgtgtgttttaataattctctatcttttattttcggaggcaaggtctcttgtttaatgtggagctcacagagtcagccatacttgct

(19) United States
(12) Patent Application Publication
(10) Pub. No.: US 2023/0078089 A1
(43) Pub. Date: Mar. 16, 2023

(54) Regulating Activation of Fibroblasts to Prevent Fibrosis

(71) Applicants: THE J. DAVID GLADSTONE INSTITUTES, A TESTAMENTARY TRUST UNDER THE WILL OF J. DAVID GLADSTONE, San Francisco, CA (US); The Regents of the University of California, Oakland, CA (US)

(72) Inventors: Michael Alexanian, San Francisco, CA (US); Arun Padmanabhan, Oakland, CA (US); Deepak Srivastava, San Francisco, CA (US)

(21) Appl. No.: 17/799,851

(22) PCT Filed: Mar. 2, 2021
(86) PCT No.: PCT/US2021/020372

§ 371 Date: Aug. 15, 2022

Related U.S. Application Data
(60) Provisional application No. 62/984,103, filed on Mar. 2, 2020.

(51) Int. Cl. C12Q 1/6883

(52) U.S. Cl. CPC C12Q 1/6883; C12Q 2600/136; C12Q 2600/158

(57) ABSTRACT

Described herein are methods for treating cardiac conditions that include modulating Meox1 enhancer activity, Meox1 transcription, Meox1 translation, MEOX1 protein function, or a combination thereof. Also described herein are methods for identifying agents that can modulate Meox1 enhancer activity.

### CROSS-REFERENCE

This application claims the benefit of priority to the filing date of U.S. Provisional Application Ser. No. 62/984,103, filed Mar. 2, 2020, the contents of which are specifically incorporated herein by reference in their entirety.

### GOVERNMENT SUPPORT

This invention was made with government support under R01 HL17240 awarded by the National Institutes of Health. The government has certain rights in the invention.

### INCORPORATION BY REFERENCE OF SEQUENCE LISTING

A Sequence Listing is provided herewith as a text file, “2121211.txt” created on Mar. 1, 2021 and having a size of 61,440 bytes. The contents of the text file are incorporated by reference herein in their entirety.

### BACKGROUND

Heart failure (HF) is a major cause of mortality for which current therapies have limited efficacy, representing a significant unmet need. Stress-activated signaling cascades can converge on the chromatin regulatory apparatus to aggravate or precipitate heart failure, triggering broad shifts in transcriptional and cell states, leading to events that fuel a cycle of pathological cardiac remodeling.

### SUMMARY

Described herein are methods for improving cardiac function that involve, for example, inhibiting Meox1 transcription, Meox1 translation, or MEOX1 protein function. As illustrated herein, Meox1 regulatory elements become activated in fibroblasts during stressful cardiac events leading to increased levels of Meox1 and a cascade of profibrotic events that exacerbate the cardiac conditions. Inhibition of such Meox1 regulatory elements can improve cardiac function.

Methods are described herein that involve contacting at least one test agent with a population of cells to provide a test assay mixture and measuring Meox1 levels to thereby identify one or more Meox1 modulating agents. For example, the population of cells can include fibroblasts, activated fibroblasts, resting fibroblasts, myofibroblasts, activated myofibroblasts, or a combination thereof. The population of cells can be from various tissues, such as heart, lungs, liver, kidney, or a combination thereof. The population of cells that is evaluated with the test agent can be from a patient seeking treatment for a heart condition or disease. The patient providing the population of cells exhibits to be tested can have increased Meox1 levels in cardiac fibroblasts, increased nascent Meox1 levels in cardiac fibroblasts, increased chromatin accessibility in a Meox1 enhancer, within cardiac fibroblasts, or a combination thereof.

In some cases, measuring Meox1 levels includes measuring chromatin accessibility of a Meox1 enhancer, measuring Meox1 transcript levels, measuring nascent Meox1 transcript levels, or a combination thereof. Measuring Meox1 levels can include measuring absolute numbers of observed Meox1 transcripts or Meox1 nascent transcripts per gene per cell. The Meox1 enhancer can be on human chromosome 17 between about positions 43,589,381 and 43,595,263. Various test agents can be tested. For example, at least one of the test agents can be an antisense oligonucleotide, a small interfering RNA (siRNA), a small hairpin RNA (shRNA), a CRISPR guide RNA, a CRISPR ribonucleoprotein comprising a guide RNA and a cas nuclease, or a combination thereof. One or more of the Meox1 modulating agents that can modulate Meox1 levels can reduce Meox1 levels, reduce Meox1 enhancer activity, or a combination thereof. For example, one or more of the Meox1 modulating agents can reduce chromatin accessibility of a Meox1 enhancer, reduce Meox1 transcript levels, reduce nascent Meox1 transcript levels, or a combination thereof. Such methods can further include administering one or more of the Meox1 modulating agents to an animal model of a condition or disease and determining whether one or more of the Meox1 modulating agents reduces the symptoms or severity of the condition or disease to thereby identity a therapeutic agent.

In addition, the methods can also include administering one or more of the test agents or therapeutic agents to a subject. Such a subject can have or be suspected of having cardiac fibrosis, lung fibrosis, liver fibrosis, kidney fibrosis, heart failure, congestive heart failure, myocardial infarction, cardiac ischemia, myocarditis, arrhythmia cardiomyopathy, dilated cardiomyopathy, coronary artery disease, hypertension, valvular heart disease, hypertrophic cardiomyopathy (HCM), familial dilated cardiomyopathy (FDCM), restrictive cardiomyopathy (RCM), arrhythmogenic cardiomyopathy (AVC), unclassified cardiomyopathy, or a combination thereof.

Also described herein are methods that involve contacting cells with an agent that inhibits Meox1 RNA transcription, Meox1 chromatin accessibility, Meox1 RNA processing, or Meox1 translation. The cells can include fibroblasts, myofibroblasts, activated fibroblasts, activated myofibroblasts, or a combination thereof. The population of cells can be from various tissues, such as heart, lungs, liver, kidney, or a combination thereof.

The agent can knock down or knock out Meox1 transcription, knock down or knock out Meox1 enhancer activity, or a combination thereof. For example, the agent can include one or more inhibitory nucleic acids, one or more guide RNAs, one or more cas nucleases, one or more cas nuclease: guide RNA ribonucleoprotein complexes, or combinations thereof. Such contacting of the cells can occur in vitro. In some cases, the modified cells can be administered to a subject with a condition or cardiac disease. The cells contacted in vitro can be allogenic or autologous to a patent or subject later administered the modified cells.

The contacting cells with a test agent or a modulating agent can occur in vivo by administering the agent to a subject. Such a subject can have or be suspected of having cardiac fibrosis, lung fibrosis, liver fibrosis, kidney fibrosis, heart failure, congestive heart failure, myocardial infarction, cardiac ischemia, myocarditis, arrhythmia cardiomyopathy, dilated cardiomyopathy, cardiac artery disease, hypertension, valvular heart disease, hypertrophic cardiomyopathy (HCM), familial dilated cardiomyopathy (FDCM), restrictive cardiomyopathy (RCM), arrhythmogenic cardiomyopathy (AVC), unclassified cardiomyopathy, or a combination thereof.

### DESCRIPTION OF THE FIGURES

FIG. 1 shows a map of the Meox1 chromosomal locus.

FIG. 2A-2J illustrate that the dynamic reversibility of heart failure with Bromodomain and Extra-Terminal Domain (BET) inhibitor exposure correlates with myofibroblast cell state. FIG. 2A graphically illustrates left ventricle (LV) ejection fraction (EF) in sham-treated mice (Sham), vehicle-treated mice with induced myocardial infarction (MI-Veh) and in CPI456-treated mice with induced myocardial infarction (MI-CPI456) as quantified by echocardiography (n=17, 21 and 21 for Sham, MI-Veh and MI-CPI456). The myocardial infarction (MI) model involved induced heart failure by a permanent anterior wall myocardial infarction. The BET inhibitor CPI456 (10 mgk) is a JQ1 derivative that was intermittently dosed as illustrated in the timeline above the graph. Statistical significance is shown between MI-Veh and MI-CPI456. FIG. 2B illustrates the left ventricle (LV) ejection fraction (EF) quantified by echocardiography in sham-treated mice (Sham), vehicle-treated Transverse Constriction Model (TAC) mice (TAC-Veh) and in Transverse Constriction Model (TAC) mice treated with the small-molecule BET-inhibitor JQ1 TAC JQ1 (n=4, 6, 6 and 6 for Sham, TAC-Veh, TAC JQ1 and TAC JQ1 withdrawn) in with intermittent dosing of BET inhibitor JQ1 (50 mgk) (no withdrawal of JQ1 vs. withdrawal of JQ1). Statistical significance is shown between TAC JQ1 and TAC JQ1 withdrawn. FIG. 2C schematically illustrates the experimental workflow for generating single cell RNA sequencing samples and Transposase-Accessible Chromatin (ATAC) sequencing samples from heart samples. FIG. 2D shows a uniform manifold approximation and projection (UMAP, Stratton et al. Circ. Res. 125, 662-677 (2019)) plot of all captured cells in the adult mouse populations colored by cluster identity. Total cells n=35,551. As shown, single cell RNA sequencing identifies the major non-cardiomyocyte populations in the adult heart. FIG. 2E shows a uniform manifold approximation and projection (UMAP, Stratton et al. Circ. Res. 125, 662-677 (2019)) plot of all captured cells in the adult mouse populations colored sample identity. Total cells n=35,551. Global transcriptional changes in hearts subjected to BET inhibition are illustrated. FIG. 2F is a dot plot showing expression (avg.exp.scale) and cell percentage of top differentially expressed (DE) marker genes between samples. FIG. 2G shows a UMAP plot of fibroblast (FB) subclusters colored by sample identity. Total cells n=13,937. FIG. 2H shows Periostin (Postn) expression in fibroblasts (FBs) within the samples illustrated as an UMAP feature plot and as a violin plot (y axis is normalized UMI levels). FIG. 2I shows an UMAP plot of FBs subclusters colored by cluster identity with a tree diagram showing cluster relationships. Representative top Gene Ontology (GO) terms for clusters 0,1,4; 2,3; and 5 are shown to the right. For FIGS. 2A and 2B, *P<0.05 and ****P<0.0001 for indicated comparison. Data are shown as means±SEM. FIG. 2J shows a schematic highlighting the approach to integrate scRNAseq with scATACseq. See extended methods for details.

FIG. 3A-3J illustrate that the reversibility of fibroblast chromatin states reveals novel dynamically accessible DNA elements that correlate with heart function. FIG. 3A graphically illustrates chromatin accessibility of distal elements in fibroblast cells derived from scATAC-seq samples. Trimming of 10% most extreme points was performed for better visualization. FIG. 3B illustrates the dynamic accessibility of distal elements in fibroblasts clustered by trend across samples (left) with top three GO terms for nearest genes to distal elements in each cluster (right). FIG. 3C illustrates enrichment scores for transcription factor (TF) motif accessibility in distal elements between samples for the ten most expressed TFs observed during TAC in fibroblasts. FIG. 3D shows a heatmap of PROSeq coverage of differentially transcribed distal regions between Unstimulated (Unstim) and TGFβ-treated fibroblasts. TOP GO terms are shown to the right with average signals for 2 replicates of each condition is shown. FIG. 3E graphically illustrates PROseq coverage measured in Unstim and TGFβ-treated fibroblasts in vitro for scATAC peaks opening (n=8964) or closing (n=1628) between Sham and TAC in vivo. FIG. 3F illustrates the effects of CRISPRi targeting of the Peak11 region upon Postn expression when fibroblasts are stimulated or not stimulated (Unstim) with TGFβ. Postn expression was detected by qPCR in the Unstim and TGFβ-treated fibroblasts in the control line and in the Peak11-CRIPRi-targeted line (each panel was normalized to its Unstim condition). FIG. 3G is a schematic illustrating correlation analysis between LV ejection fraction and chromatin accessibility—highlighting a negative or positive correlation. FIG. 3H is a volcano plot showing correlation coefficients (referred to analysis depicted in FIG. 3G) and corresponding p-values of 470 superenhancers in fibroblasts. A region distal to the Meox1gene has one of the most negative correlation coefficients. For FIG. 3F, **P<0.01 and ****P<0.0001 for indicated comparison. Data are shown as means±SEM. FIG. 3I graphically illustrates chromatin accessibility at distal elements in myeloid cells. FIG. 3J graphically illustrates chromatin accessibility at distal elements in endothelial cells. Trimming of 10% most extreme points was performed for better visualization in FIGS. 3I-3J.

FIG. 4A-4K illustrate chromatin accessibility and nascent transcription of a cis-regulatory element controlling Meox1 expression. FIG. 4A shows an UMAP plot of fibroblasts subclustered-colored by sample identity (same as for FIG. 3G) and Meox1expression in fibroblast in the samples shown as UMAP feature plot and violin plot (y axis is normalized UMI levels). FIG. 4B illustrates the Meox1 locus (gene and enhancer) showing from top to bottom: coverage of scATAC samples in fibroblasts; ChIPseq for BRD4 (GSE46668), H3K27Ac and CTCF (ENCSR000CDF and ENCSR000CBI) in the adult heart; coverage of PROseq in Unstim and TGFβ-treated fibroblasts; and co-accessibility measures between Meox1 promoter and Peak9/10 region in fibroblasts using scATAC. A highly transcribed region (Peak 9/10) is highlighted in red within the large Meox1enhancer. FIG. 4C illustrates chromosome conformation capture (4C) between the Peak9 region (anchor point) and Meox1promoter showing 4C coverage in Unstim and TGFβ-treated fibroblasts. 922 kb (top) and 328 kb (bottom) genomic regions are shown. Last track represents the called TGFβ-induced loops with Peak9 (colored in purple in the original). FIG. 4D shows a schematic illustrating CRISPRi targeting of three regions within the Meox1enhancer (Peaks 5, 9 and 13) at the top. Meox1 expression by qPCR between Unstim and TGFβ-treated fibroblasts in the three CRISPRi fibroblast lines targeting Peak 5, 9 or 13 (each panel is normalized to its Unstim condition). For FIG. 4D, **P<0.01 and ***P<0.001 for indicated comparison. Data are shown as means±SEM. FIG. 4E illustrates chromatin accessibility at the Meox1 super enhance (SE) in fibroblasts, myeloid cells, and endothelial cells that were sham-treated (left-most bar), subjected to TAC (left-center bar), subjected to TAC and treated with JQ1 (right-center bar), or subjected to TAC and treated with JQ1 for a time followed by JQ1 withdrawal (rightmost bar). FIG. 4F illustrates scATAC coverage between samples at the Meox1 super enhancer within fibroblasts identified multiple dynamic peaks during heart failure with pulsatile BET inhibition. FIG. 4G graphically illustrates Meox1 expression as measured by qPCR in Unstim and TGFβ-treated fibroblasts FBs, with or without JQ1 treatment. FIG. 4H graphically illustrates that Peak9/10 is the essential regulatory element controlling Meox1 expression as shown by deletion of Peak9/10 in cardiac fibroblasts. Meox1 expression was measured by qPCR of CRISPR Cas9 treated WT (isogenic line) and Peak9/10 deleted cells that were unstimulated (Unstim) and stimulated with TGFβ. FIG. 4I graphically illustrates Brd2, Brd3 or Brd4 expression as measured by qPCR of individual BET genes in Unstim or TGFβ-treated fibroblasts with siRNA targeting either Ctrl, Brd2, Brd3 or Brd4. FIG. 4J graphically illustrates Meox1expression as measured by qPCR in Unstim or TGFβ-treated fibroblasts with siRNA targeting either Ctrl, Brd2, Brd3 or Brd4. For 4I-4J, *P<0.05, ***P<0.001 and ****P<0.0001 for indicated comparison. Data are shown as means±SEM. Data are shown as means±SEM. FIG. 4K illustrates that deletion of Brd4 improves cardiac function during heart conditions.

FIG. 5A-5J illustrate that MEOX1 is a novel regulator of fibroblast plasticity and profibrotic function. FIG. 5A shows representative images of fibroblasts seeded on compressible collagen gel matrices and assayed for gel contraction after treatment with TGFβ and siRNA targeting Meox1 for 72 h. For comparison, the effects of a control siRNA on collagen gel contraction is also shown. FIG. 5B graphically illustrates quantification of the gel contraction images, as reported as percentage contraction; (n=4 plates per condition). Data are presented as mean±SEM. FIG. 5C graphically illustrates quantification Edu incorporation in fibroblasts after treatment with TGFβ and a Ctrl siRNA or a Meox1-targeted siRNA for 72 h. Data are presented as mean f SEM. For FIGS. 5B and 5C, **P<0.01 and ****P<0.0001 for indicated comparison. FIG. 5D is a heatmap of MEOX1-HA ChIPseq occupancy at protein coding gene (−2 kb from transcriptional start site, +2 kb from transcriptional end site) sorted by the strength of ChIPseq signal (8366 regions shown). FIG. 5E is heatmap of PROseq coverage of differentially transcribed protein coding genes between TGFβ-treated fibroblasts with Ctrl or Meox1 siRNA. Average signal for 2 replicates in each condition is shown. Top related GO terms and example genes are shown to the right. FIG. 5F illustrates coverage of MEOX1 ChIP and PROseq (Unstim and TGFβ-treated fibroblasts with Ctrl or Meox1 siRNA) at the Ctgf or Postn locus (including the PostnPeak11 regulatory element). FIG. 5G graphically illustrates Meox1 expression in human cardiac disease, for example hypertrophic cardiomyopathy (HCM) and dilated cardiomyopathy (DCM). Bulk RNAseq data is shown of human MEOXlexpression between controls and individuals with HCM/DCM (-GSE141910) assessed in heart tissues. FIG. 5H graphically illustrates Meox1 expression in idiopathic pulmonary fibrosis (IPF). Bulk RNAseq data is shown of human MEOXlexpression between controls and individuals with Idiopathic pulmonary fibrosis (-GSE134692), assessed in lung tissues. For FIGS. 5G-5H, p-values are indicated in the panels. FIG. 5I graphically illustrates expression of Meox1 in human fibroblasts from lung, liver and kidney. MEOX1 expression was measured by qPCR in unstimulated fibroblasts (Unstim, left bars), as well as in TGFβ (middle bars) or TGFβ+JQ1 (right bars) treated human fibroblasts from lung, liver and kidney. Data are shown as means±SEM. FIG. 5J schematically illustrates the transcriptional switch that activates fibroblasts correlates with heart disease state. Combining single cell transcriptomic and epigenomic interrogation, the inventors discovered key enhancers and protein coding genes that dynamically regulate fibroblast plasticity and profibrotic function, including the transcription factor MEOX1.

### DETAILED DESCRIPTION

As illustrated herein, Meox1 enhancer elements are fibroblast-specific transcriptional switches that reversibly mediate stress-induced fibroblast activation. Experiments described herein show that inhibition of Meox1 expression and function, and/or inhibition of the Meox1 enhancer can improve cardiac function, lung function, kidney function, liver function, or a combination thereof. This application therefore relates to methods of inhibiting Meox1 transcription, inhibiting activation of Meox1 enhancer, or a combination thereof. Also described are screening methods useful for identifying agents that can modulate Meox1 transcription and/or Meox1 enhancer activity.

For example, using a combination of mouse models of heart failure, small-molecule BET bromodomain inhibitors and single-cell omics, the inventors have demonstrated that cardiac myofibroblasts are exquisitely sensitive to transcriptional inhibition. For example, myofibroblasts exhibit robust reversibility of their cell states, switching between basal fibroblasts and activated myofibroblasts in a manner that directly relates to BET inhibitor exposure. In addition, the data provided herein also shows heightened Meox1 expression in activated fibroblasts from human lungs, kidneys, and livers. Leveraging integrated epigenomic approaches, the inventors discovered and dissected the function of a super enhancer that regulates the expression of the transcription factor Meox1. By modulating this super enhancer, Meox1 expression can modulated to reduce the adverse effects of fibroblast activation.

Meox1 is specifically expressed in fibroblasts and controls their proliferation and contractile activity by directly binding the promoter of fibroblast genes. As described herein, modulation of transcription during disease pathogenesis, coupled with single cell interrogation, uncovered cell states and molecular mechanisms involved in the progression and reversal of chronic diseases, pointing to new therapeutic approaches.

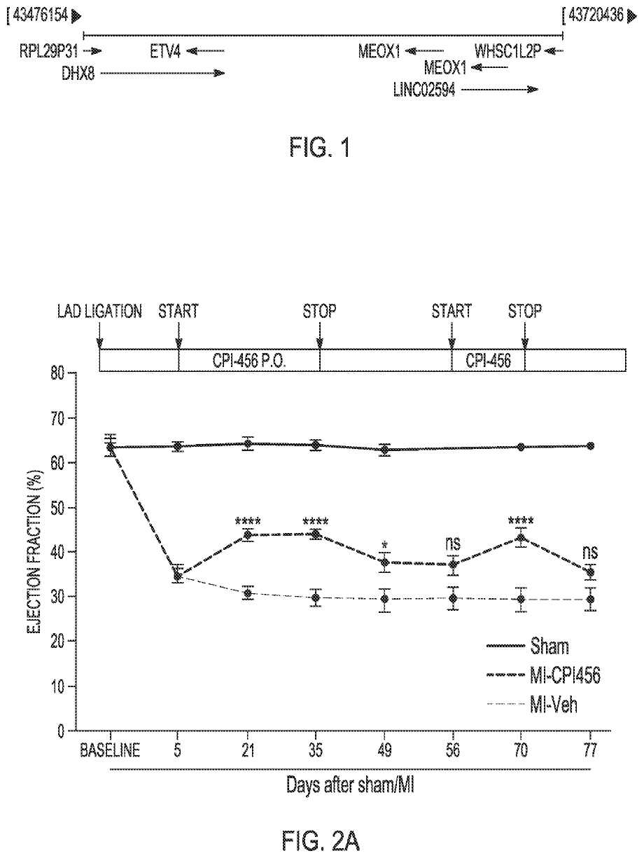

The human Meox1 gene is on chromosome 17 and is located at NC_000017.11 (43640389 . . . 43661977, complement). A map of the Meox1 chromosomal locus is shown in FIG. 1.

An example of a MEOX1 amino acid sequence is available from the NCBI database as accession number NP_004518.1 and shown below as SEQ ID NO:1.

```
    1 MDPAASSCMR SLQPPAPVWG CLRNPHSEGN GASGLPHYPP
  41 TPFSFHQKPD FLATATAAYP DFSASCLAAT PHSLPQEEHI
  81 FTEQHPAFPQ SPNWHFPVSD ARRRPNSGPA GGSKEMGTSS
121 LGLVDTTGGP GDDYGVLGST ANETEKKSSR RRKESSDNQE
161 NRGKPEGSSK ARKERTAFTK EQLRELEAEF AHHNYLTRLR
201 RYEIAVNLDL SERQVKVWFQ NRRMKWKRVK GGQPISPNGQ
241 DPEDGDSTAS PSSE
```

A nucleotide sequence for the above human MEOX1 protein is shown below as SEQ ID NO:2.

| | |
|---|---|
| 1 | AGCCCTCTGC AGGCATTTGC TCTGGGCTCC AGGACGAACT |
| 41 | CCTCGTCAGC TGGTGCGTCT GGGTTGGGAC AGTGAAAATG |
| 81 | TTTAGTGTCA TTGGCGACAA ATACACATAC AGGGGATCCC |
| 121 | AGTAGGTAGG CTGGTGCATT GGAGGGGCAG GAGCAAGCCC |
| 161 | GACATAGGTG TGTGCACACA CATAGGAGTA GCTGGCCACA |
| 201 | TGTGTGTGTA CTTTAAGGAG AGACTTTAGT TTTGGGTTTT |
| 241 | TTTTTTTTTT TGGTTCCTGG GGTAAATTTT CTGTTGAACA |
| 281 | TTTTTCCCTC CTATTTAGTT TTTTTCTTTT TGCATTTTTA |
| 321 | AAAATTTGAA CATAAAAAGT ATAAAGAATC AAATCTTTGA |
| 361 | AAGGACCGAG GCGTGCAGCG GACAGCAGAT GGATCCCGCG |
| 401 | GCCAGCAGCT GCATGAGGAG CCTCCAGCCC CCAGCCCCTG |
| 441 | TCTGGGGCTG CCTTCGAAAC CCCCACTCGG AAGGGAATGG |
| 481 | GGCCTCAGGG CTACCCCACT ACCCGCCCAC CCCGTTCTCC |
| 521 | TTCCACCAGA AACCAGACTT CCTGGCGACA GCGACGGCAG |
| 561 | CGTACCCTGA CTTCTCAGCC TCCTGCCTGG CAGCCACCCC |
| 601 | ACACAGCCTG CCCCAGGAGG AGCACATCTT CACTGAGCAG |
| 641 | CACCCCGCTT TCCCACAGTC CCCCAACTGG CACTTCCCTG |
| 681 | TCTCAGACGC CCGGCGCAGG CCCAACTCAG GCCCGGCAGG |
| 721 | GGGTTCCAAG GAAATGGGGA CCAGCAGCCT GGGCCTGGTG |
| 761 | GACACCACAG GAGGCCCAGG CGATGACTAC GGGGTGCTTG |
| 801 | GGAGCACTGC CAATGAGACA GAGAAGAAAT CATCCAGGCG |
| 841 | GAGAAAGGAG AGTTCAGACA ACCAGGAGAA CAGAGGGAAG |
| 881 | CCGGAGGGCA GCAGCAAAGC CCGCAAGGAG AGGACGGCCT |
| 921 | TCACCAAGGA GCAGCTGCGA GAGCTGGAGG CAGAGTTTGC |
| 961 | CCATCATAAC TACCTGACTC GGCTCCGCAG ATATGAGATT |
| 1001 | GCGGTAAACC TGGACCTCTC TGAGCGCCAG GTCAAAGTGT |
| 1041 | GGTTCCAGAA CCGAAGGATG AAGTGGAAGC GTGTGAAGGG |
| 1081 | AGGTCAGCCC ATCTCCCCCA ATGGGCAGGA CCCTGAGGAT |
| 1121 | GGGGACTCCA CAGCCTCTCC AAGTTCAGAG TGAGATTCTG |
| 1161 | CATGGAGGAA AAATGACTAA GGACTGAGCC CCCTACCCAA |
| 1201 | CTACCCCCAC CCCAATCCCA CCTTCACCCT CTTCCTTCCC |
| 1241 | CAGCCAGGGC AGCCTCTCCA CATCTTTCCC TGACTCTTGG |
| 1281 | ATATGAAACT GCCCAGCATT CCTGGGAGTC TTAGGATTTT |
| 1321 | L1AGGAAGT1 CTGTCCAGCC TCTTAGCAGC CTCTTCCCTA |
| 1361 | GGGCCTTTGC TCCCACACTC TCATGGAATC AGACAGAGAT |
| 1401 | CCTACCGGGC CGGATGAATC TGGAAACAGC TTCAGAGATA |
| 1441 | CTGCTTCTCA GCGTCTCTTG GCTGCCACCC ATGCCTCCTC |
| 1481 | CTACCGCTGT TCTCCTAGGT CAGCCAGGCC TCCTCCTGGT |
| 1521 | CTGGACACCA CCTGGCCTGG TGGGAGAGGA GCTTTGGAAC |
| 1561 | CAGCTGGCGA CTCGGAAAGT AAATGCTTCA AAAGGAAGGA |
| 1601 | AATGACAGAG ACACACGCCC TTGCCCACCI TCCTCTGTAG |
| 1641 | GCTGCACATC TGAGGCTTTG GGGCCCCTTA GTTGTCCCGA |
| 1681 | AACCCCAAGA AAAATCAGAA TGAGGAGAGT CAAGGACAGC |
| 1721 | AACTCAGCTG CTGCAAGCCA GAAACACATC CCTGTCTCCA |
| 1761 | AATTTGTTGG CTAAGTGGAG ACACTTCTGA GAACTGACTA |
| 1801 | GAGAAGACAG AAAAATAGCC CGATGTAGGT TTCGGTGTCC |
| 1841 | CCATATAGGC CCGTCCACAC AGGCTTGACT GGGTGGACAA |
| 1881 | GAATGAACCC ATGACAGCAC CTGCTGCTTC AAAATCAAAA |
| 1921 | TCAATTTAGG GATACAGCAG GGGCTGTTGG GCTGTGCTCC |
| 1961 | AGAGAAAAGG AGCAGCTAGT CCTTTTAAAT CCACGATTTC |
| 2001 | TGGATTGAAA ACCTGTCCAG ATGCTGAGTT GTTGGGCTGA |
| 2041 | ACAACTAGGA GCTGAAAACA ACGTAGAGGC TGGAAAGTGT |
| 2081 | CCCCTGCATT CTGGAGGGGA GGGGAGATAA TAAGGAGGGC |
| 2121 | TGCTGGGTGA GGGCCTGGAG ATGTGGAACC CTGGAGTGGA |
| 2161 | AGGTTCTCCA GTGAGAGTGT CCTGTGACTG CAAAAGGGGA |
| 2201 | CAAGAAAATC CCTCTTCCTC CATGGGATGG ATTTAAGCTC |
| 2241 | TTGCTGTGTG TTCTACAAAT GCTGTTATTG TGGGAGGAAA |
| 2281 | TGCTAGGTTT TTGTGTGTGG ACTGCCCAGA CCTCAGCCAG |
| 2321 | GTCTTCTGGA GATGACATTT GAGGACTGAT GGCCAAAGAG |
| 2361 | CATGGGGGAC TGAAGCCCTG GCTGCCTCAG CGCTCTGTCT |
| 2401 | CCCAACACCA GCTGGTGTTG CAGAGGGAGG TCAAGGTGAG |
| 2441 | TTTGGATCTC TTGTACGCAG ATGTAATCAT TCACATGTAA |
| 2481 | AAATAACCCC ACCTCCCCAC CCCAAAAAGG GCAAGAGCTG |
| 2521 | TGGAAAATGA TTGCCAAATG AGATGGCTGG TTAGAGCATG |
| 2561 | ATTTTTTCTA AAGCATACTT CATATATTTT CTTAAGATTA |
| 2601 | CATCAAGCTA ATTGTGCGAG CTCAATTCAC TTTGTAAGAA |
| 2641 | AACTCTCGGA GAAATAAAAT CAATAAAAAG CAAA |

Other human Meox1 sequences are available with accession numbers NM_013999.3 (GI: 84105330); NM_001040002.2 (GI: 1675087437); and XM_011524818.2 (GI: 1370470996).

An enhancer regulating the expression of the Meox1 transcription factor is present on human chromosome 17 at about positions 43,589,381 to 43,595,263. A sequence of the peak 9/10 region of this enhancer is shown below as SEQ ID NO:3.

```
      1 CTTGCAATCC CAGCACTTTG GGAGGCTGAG GCGGGTGGAT
    41 CAGCTGAGGT CAGGAGTTTG AGACCAGCCT GCCCAACATG
    81 GTGAAAACCC ATCTCTACTA AAAATATAAA AACTAAAGGC
  121 GGGCGTGGTG GTTCGTGCCT GTAAGCCCAG CACTTTGGGA
  161 GGCTGAGGCA GGAGGATCAC AAGGTCAGAA GATCGAGACC
  201 ATCCTGGCTA ACATGGTGAA ACCCCGTCTC TAGTAAAAAT
  241 ACAAAAAATT AGCCTGGTGT GGTGGCGGGC GCCTGTAGTC
  281 CCAGCTACTC AGGAGGCTGA GGCAGGAGAA TGGCGTGAAC
  321 CCAGGAGGCG GAGCTTGCAG TGAGCCGAGA TCGCGCCATT
  361 GCACTCCAGC CTGGGTGAGA GTGTGAGACT CTGTCTCAAA
  401 AAAAAAAAAA AACCATATAT ATATATATAG TTTATATATA
  441 TGGTTTATAT ATATAGTTTA TAGTTTATAT ATATGGTTTA
  481 TATATATAGT TTTTATATAT ATAGTTTATA TATACATATA
  521 TATATAAACT AGCCGGGGGT GGTGGTGGAT GCCTGTAATC
  561 CCAGCTACTC GGGAGGCTGA GGCAGGAGAA TTGCTTGAAC
  601 CCAGGAGACG GAGGTTGCAG TGAGCCAACA CGGTGCCACT
  641 GGACTCTAGC CTGGGTGAGA GAGTGAGACT CTGTCTCAAA
  681 AAAAAAGAAT AATTTTTTCT TTAACAGAAC AAATTGCCTC
  721 GCTGAGTCGG ACCAGCTGCC CGTGGATCCT ACGCTGCAGT
  761 GACACCAGAC TCCTCATGTT GTGGGGTAAG TGTGTCCCCT
  801 TTCTTTCCTG CTTCTGTCTT TATACACACA GTCCCTTTCC
  841 TTTGATACGT TTTCTCCTCT CGTCTGCCTC TGAGCCAGCC
  881 CCAAGAGTCT TCTCCGTGCT TCCTGACTCC TCTCTGCTTA
  921 GCTGTTACCC CATTTGTTCC TCTGCACCAT AGTTAAACCT
  961 ATTTAGAGGT CTGTGTCCCC CACCAAACTG TGAGCTCCTT
1001 GAAGGCAGGC GATGTCCATT TTGTATCCCC CCGAAAACCA
1041 TCCCCCTACC CCCAGTACAG ACTCTGGCAC AAAATAGGCA
1081 TTAAGTAAAT GAATGAAGGA ATGAATGGAT GGAAAGGATG
1121 CCATTGTAGG GGGGAAAGAG TAATATCTAT TACTTTTATT
1161 TAAAAAAAAC TTTTAATTTC CACTTCTAGG CATATATCTT
1201 TTCCTTATCT GTCACAAAGT TCATGGCTTG AGATCCCTAT
1241 AACAAAAGAT AGATTAACAA GAGAAAAGCA TACACATTTA
1281 TATAATAAGT TTCCGTGACA TGGGAGATGA AGACCCAGGG
1321 AAATAGGGAA TACTGTGTAT ATTTTATGCT TAGGTTTAAT
1361 GAGGAGTAGA GCGTTGTGTA GAAACATGAT GGACGAGGCC
1401 AGGCGTGGTG GCTCACGCCT GTAATCCCAG CACTTTGGGA
1441 GGCCGAGGCG GGTGGATCAC TTGAGGTCAG GAGTTCAACA
1481 CCAGCCTGGC CAACAGGATG AAACCCCGTC TCTACTAAAA
1521 ATACAAACAA TTAGCTGGGC ATGGTAGCGC ACGCCTGTAA
1561 TCCCAGCTAC TCGGGAGGCT GAGACAGGAG AATTGCTGGA
1601 ACCCAGGAGG TAGAGGTTGC AGTGAGCGGA GAACACACTA
1641 CTGCACTCCA GCCTGGGAGA CAGAGCGAGA CTCCATCTCA
1681 AAAAAAAAAA AGAAGAAGAA ATATGATGGA AGAAAGGGGG
1721 TATGATCTAA TAGTAATCAA CTTGGGGGAG CTTAGCAAGG
1761 CCTGTTTCTT CAGATTCTTC TCTGCTTCTT CATTCCTTTC
1801 CTCTGGATAT AGGGAGGACC CCTCTGGAAT GAGGGTCTTA
1841 TGACCTACTT TAGAAGAAAG ACAGAAAATT CTTTCATGAT
1881 CTGCTTCAGG GGAGAATAGT AGCAGCAAGT CAGAGAGATC
1921 TTCCTCCTTC TGCTGCTTTT GCCGAGGTGC TGTATTTTGG
1961 GGTAGCATGT CCTGAACTCC ATCACCATCT TGTTTGCCCC
2001 TTTAGGATCT GCCAGCAAGA TTTTGAGCAA AATCCTAATC
2041 TCTGGCCTCA TTTTAGAGCT TGTAGCATCA GGTTTGGAGG
2081 CTAGGGGTTC CTGGTGCCCC AGCACCAGAG AGGAGGGTGA
2121 GAAGGCCACC TTGGGAGAGC TGGGCCATTT CAGGGGAGGA
2161 AATTAAAACT AAATTGCATT AATTGGTTTT CATATTGAAC
2201 CCTGCTTCTT AAGTGTGCTA AATGTCTGGT TAAACAAATA
2241 GCTAAAAAGA TAGAATCACT TTGCTATCAT TTTTTGTGGT
2281 AAGGGAAAAG ACAAAAAAAC AAAGATGTTG ATTTAAGATA
2321 TAGCCTAGGG CTGGGGGGCT GGGGGAAACC TGGTGAAGGC
2361 TCCTGTTGTT TGGGGTCGGA GGTCGCAGGA GCAGCGGCTG
2401 CCTGGGGATC AGGCTCAAGG TTCTGGAATG ATGGAAAAAC
2441 CTGGCCTGAC GCTTTTCCTC AGCGGACCCT TCACTGGCTA
2481 ACAGGAGCCA GCTGAGCGAA CACAGAGGCG CTGTAACCGG
2521 CGCAGATCCC AGCTTCCTCT GAAATTTCCA GGGCTCTTTT
2561 TTCCTGTCGG ATTCCAGACA GATGAAATTT TCCTGGCCCC
2601 GGCCCATTCC TGGCAGCATC TCCCCTCTGA ATCATCATAA
2641 ATCAGGGCTT GGCGGGGAGC GGTGGGATTT TCCTGATTTT
2681 CCTGATCGAC ACGCTGCTGC CCTGGTAAAA TGGGGCTCCG
2721 TTTCCAAGGC TAAAAATAGC TCTGGGGTGC TTGCCTGAGT
2761 TCTCCCCATC GCAAGTTGCT GCTTCGTATT GTAAATATTC
2801 ATATCTGTCT ATGATTATTA TTTCATCGAG AAGGGCTGTG
2841 GACGTGCAGA TGCGGGCCGC TGGGGGCCTC GGTGGTCTGA
2881 AGTGACACCA TTGTTCACAG GATCATATGC GGGGGCCTGT
2921 GCATTCCTCA AAGCCTCCAT CATCCAAGGA GCCCCCAATT
2961 AATTTCAATA CAAACACCGG ATATGGCCTC CCCCGCGCCT
3001 TTGCAGCCAT AACACATGAG GTCATGCTGC TTTGGTTAAC
3041 CCGAGGAGTT GCAGAGTGAG ATGGGGAGGC TTCTAGGCCC
3081 CAGGGGAGGC CTGGGGTATC TGCCCTGCCT GGTTTAGGCG
3121 AGGCTGTTTA CTGCAATGGG GTCTACGGCA CACCCACACT
3161 CTCTCGCCTT CCTTCACTCA GAAACTGAAA TCTCTGATTC
3201 CCGATGTCTC TTCCCAGCCA CTCTCCCCAT CTCTAGCCAC
3241 TTGGCTGCCA ACCCACCACC CACCCCTGTG GTTCTTTGCA
3281 AACTCCTTAC CCTCCGTGCT CTTGCATACA CAGCTTCATT
3321 CACCTGGGGC ATCTTTTTAC CTCTTCATCA GGCTCCTTCT
3361 GGAAGATTCA CCTCAGGAGT TTCTTCCTTC AGGAAGCATT
3401 TCCTGATCAC TCCATTCAGG GTTAAGTACT GTTTTCTTGG
3441 CTGGGAGCAG TGGCTCATGC CTGTAGTCTT GGTACTTTGG
3481 AAGACGGAGG CAGGCGGATC ACTTGAGCTC AGGAGTTCGA
3521 GACCAGTCTG GCCAACATGG TGAAACCACA TCTTTACTAA
3561 AAATACAAAA ATTAGCTGGG CACAGTGGTG CATGCCTGTA
3601 ATCCCAGCTA CTCGGGAGGC CGAGGCAGGA GAATCGCTTG
3641 AACACAGAAG GTGGAGGTTG CAGTGAGCCG AGATTGCGCC
3681 AATGCATTCC AGCCTGGGTG ACAGAGTGAG ACTCCATCTC
3721 AACAACAACA ACAAAATTAA ATTTAAAAAA AAAGTACTTG
3761 CTGGGCGTGG TGGCTCGCGC CTGTAATCCC AGCGCTTGGG
3801 GAGGCCGAGG CTGGTGGATC ACCTAAGGTC AGGAGTTCAA
3841 GACCAGCCTG GCCAACATGG TGAAATCCCA TCTCTACTAA
3881 AAATACAAAA AAGTAGCTGG GCATGGTGGC AGGCGCCTGT
3921 GATCCCAGCT ACTCAGGAGG CTGAGTCAGG AGAACTGCTT
3961 GAACCCGGGA GGCAGAGGTT GCAGTATGCC GAGATCATGC
4041 CATTGCACTC CAGCCTGGGC AACAAGAGCA AAATTCCATC
4081 TCAAAAAAAA AAAACTACCA CTTTCAGCCG GGCAGGGTGG
4121 CTCACGCCTA TAATCCCAGC ACTTTGGGAG GCCGAGGTAG
4161 GTGGATCACA AGGTCAGGAG ATCGAGACCA TCCTGGCTAA
4201 CACAATGAAA CCCCATCTCT ACTAAAAATA CAAAAAAATT
4241 AGCCAGGCGT GGTGGTGGGT GCCTGTAGTC CTAGCTACTC
4281 AGGAGGCTGA GGCAGGAGAA TGGCGTGAAC CTGGGAGGCG
4321 GAGCTTGCAG TGAGCCGAGA TCACTGCAAC TGCACTCCAG
4361 CCTGGGCAAC AGAGGGAGAC TCTGTCTCAA AAAAAAAATT
4401 CCACTTTCTC TTCTGTGTTC CTACAGCTTA TTGATTCTCA
4481 AACATTAGCC CTCATCAGAA TCGCATGGAG GGTTTGCTAA
4521 AACACCGATT GCTGGGCCCC TCCCAGTTTC AGAATCAATA
4561 GGTCTGAAGT AGGGCTTGCA CATTTGCATT TCTAACAAGA
4601 TCTCAGGAGA CACTGATGCT GCTACAGCCC CCTGTGATCA
4641 CTTCCTCCAT TGGTGGAACT TACCCTGCTG TGCTGCAATT
4681 ACCCGCCTTT TTCTTTTCTT TTCTTTTTTT TTTTTTTGTT
4721 TGAGACAGGG TCTCACTCTG TCACCCAGGC TGGAGTGCAG
4761 TGGCGCCATC TTGGCTCACT GCAACCTCTG CCCCTGGGTT
4801 TAAACAATTC TCCTGCCTCA GCCTCCCGAG TAGTTGGGAT
4841 TACAGGTGCC TGTCACCACA CCCGGTTAAT TTTTGTATTT
4881 TTAGTAGAGA CGGGGTTTCA CCATGTTGGC CAGGCTGGTC
4921 TCAAACTCCT GACCTCAAGT GATCCACCCG CCTCGACCTC
4961 CCAAACTGCT GGGATTACAG GCGTGAGCCA CCTGTCCGGC
5001 CACCTGCTTA TTTTTTGTTC CCTCCCACTG GGAGGGCTGG
5041 GACTGTCTTT TCCATTTCTC TATCCTACTG CTTGGCAAAC
5081 AGTGAAGCTG ATCACTGGAG GTTTGTTGAC TGAATGAATT
5121 GTGGATTTGG AACCAACCTG CTAGTTGTAG AGCTCAGTTG
5161 AGGGGAGGAG GTCTGCTGGT GAGAGGGCTG GTTCTCAGGG
5201 CTTTTGGGGT CATGAGTATG TTACCTGAAA GGGGGCCCAA
5241 TCCAGATCCC AAGAGAGGAT TCTTGGACCT TGCAGAAGAA
5281 AGAATTCGGG GCGAGTTTAT AGAGTAAAGT GAAAGCAAGT
5321 TTATTAAGAA AGTAAACGGG CTGGGCGTGG TGGCTCACTC
5401 CTGTAATTCC AGCACTTTGG GAGGCCGAGG AGGCGGATCA
5441 CCTTAGGTCA GGAGTTCGAG ACCAGCCTGA TCAATATGGA
5481 GAGACCCCAT CTCTACTAAA AATACAAAAT TAGCCGGGCG
5521 TGGTGACTCA CGCCTGTAAT CCCAGCTACT CAGGAGACTG
5561 AGGCAGGAGA ATCGCTGGAA CCCAGGAGGT GGAGGTGGCA
5601 GTGAGCCAAG ATCGCGCCAT TGCACTCCAA CCTGAGCAAC
5641 AAAAGCAAAA CTCCGTCTCA AAAAAAAAAA AAAGAAACTA
5681 AATGAATAAA GAATGGATAC TGCATAGGCA GAGCGGCGGC
5721 ATGAGTTGCT TGACTGAGTA TGCTTATTGT TCCGGTTTTT
5761 TTTTTTTTTT TTTGAGACGG AGTCTCGCTC TGTTGTCCAA
5801 GCTGGAGTGC AGTGGTGCAA TTCAGCTCAC TGCAACCTCC
5841 GCCTCCTGGT TTCTAGCAAT TCTCCTGCCT CAGCCTCCCA
5881 AGTAGTTGGG ATTACAGCTG TGCGCCACAA CATCAGGCTA
5921 ATTTTTTATA TTTTTAGTAG AGACAGGTTT TCATCATGTT
5961 GGCCAGGCTG GTCTTGAACT CCTGACCTCA AGTGATCCTC
6001 CTG
```

The Meox1 sequences can vary amongst the human population. Many such variants can include codon variations and/or conservative amino acid changes. However, the Meox1 sequences can also include non-conservative variations. For example, the Meox1 nucleic acids or Meox1 proteins can have at least 85% sequence identity and/or complementary, or at least 90% sequence identity and/or complementary, or at least 95% sequence identity and/or complementary, or at least 96% sequence identity and/or complementary, or at least 97% sequence identity and/or complementary, or at least 98% sequence identity and/or complementary, or at least 99% sequence identity and/or complementary to any of the Meox1 nucleic acid or Meox1 protein sequences described herein.

This Meox1 enhancer described herein can be detected in nascent Moex1 transcripts. Hence, the enhancer is therefore transcribed. Hence, methods of modulating both Meox1 chromosomal sites and Meox1 RNA transcripts can be used to modulate Meox1.

Inhibition of Meox1 transcription, Meox1 translation, or MEOX1 protein function can be used to treat cardiac diseases and conditions. Examples of diseases and conditions that can be treated include heart failure, cardiac fibrosis, lung fibrosis, kidney fibrosis, liver fibrosis, congestive heart failure, myocardial infarction, cardiac ischemia, myocarditis, arrhythmia, or any combination thereof.

The epigenetic acetyl-lysine reader protein BETs (Bromodomain and Extra Terminal) functions may be chromatin co-activators during heart failure pathogenesis that can be pharmacologically targeted in vivo. Administration of the small molecule BET inhibitor JQ1 can prevent and treat HF in several rodent models. However, the endogenous cell states and epigenetic mechanisms that mediate these salutary effects, and their degree of reversibility, remain unknown. Experiments described herein leverage small molecule BET bromodomain inhibition to transiently interdict enhancer-to-promoter signaling in murine heart failure models, coupled with single cell RNA-Seq and single cell ATAC-Seq of heart tissue, to discover the dynamic cell states and active chromatin elements underlying therapeutic responses.

JQ1 is a thienotriazolodiazepine with the structure shown below. It is a potent inhibitor of the BET family of bromodomain proteins. The BET family of bromodomain proteins which include BRD2, BRD3, BRD4, and the testis-specific protein BRDT in mammals.

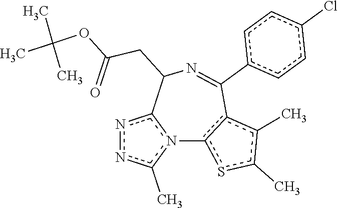

BET inhibitors structurally similar to JQ1 are being tested by various workers during clinical trials for a variety of cancers including NUT midline carcinoma.

### Screening

Agents that modulate Meox1 and thereby reduce the symptoms, severity and/or progression of heart diseases/conditions can be identified by using the methods described herein.

Such a method can, for example, involve contacting a population of cells with one or more test agents to form an assay mixture, and then measuring Meox1 levels to thereby identify one or more Meox1 modulating agents. The population of cells can include cardiac cells, fibroblasts, resting fibroblasts, myofibroblasts, or a combination thereof. In some cases, the population of cells comprises activated fibroblasts. For example, the fibroblasts can be activated by TGFβ.

Measuring Meox1 levels can involve measuring chromatin accessibility of a Meox1 regulatory element, such as an enhancer. For example, the Meox1 regulatory element can be a peak 9/10 enhancer such as the enhancer on human chromosome 17 between about positions 43,589,381 and 43,595,263.

In some cases, the screening method can involve measuring Meox1 transcript or protein levels. For example, measuring Meox1 levels can involve measuring absolute numbers of observed Meox1 transcripts (UMI counts) per gene per cell. Test agents can be selected as Meox1 modulating agents that increase Meox1 levels.

However, test agents are preferably selected as Meox1 modulating agents that reduce Meox1 levels. For example, one or more of the Meox1 modulating agents can reduce Meox1 enhancer activity. Reducing Meox1 enhancer activity can involve, for example, reducing chromosomal accessibility of a Meox1 enhancer. The Meox1 enhancer can be on human chromosome 17 between about positions 43,589,381 and 43,595,263.

In some cases, the population of cells in the test assay are from a patient seeking treatment for or prevention of a heart condition or disease. Such a patient can exhibit increased Meox1 levels in his or her cardiac fibroblasts, increased chromosomal accessibility in one or more Meox1 regulatory elements within cardiac fibroblasts, or a combination thereof.

The screening methods described herein can also include administering one or more of the Meox1 modulating agents to an animal model of a heart condition or disease and determining whether one or more of the Meox1 modulating agents reduces the symptoms or severity of the heart condition or disease to thereby identity a therapeutic agent. In addition, the methods can include administering one or more of the test agents or therapeutic agents to a patient.

### Modulation of Meox1

Meox1 can be modulated by a variety of agents and methods. For example, Meox1 can be modulated by any of test agents, therapeutic agents, inhibitory nucleic acids, guide RNAs, nucleases, a ribonucleoprotein complexes that include a cas nuclease, inhibitory nucleic acids, chromatin stabilizing agents, or combinations thereof described herein.

For example, test agents, therapeutic agents, inhibitory nucleic acids, guide RNAs, nucleases, a ribonucleoprotein complexes that include a cas nuclease, an inhibitory nucleic acid, a chromatin stabilizing agent, or combinations thereof can be administered to subjects such as patients or animals. Patients and animals receiving the test agents or therapeutic agents can be in need thereof of the one or more of the test agents, therapeutic agents, inhibitory nucleic acids, guide RNAs, nucleases, a ribonucleoprotein complexes that include a cas nuclease, an inhibitory nucleic acid, a chromatin stabilizing agent, or combinations thereof. In some cases the subject receiving the test agents, therapeutic agents, inhibitory nucleic acids, guide RNAs, nucleases, a ribonucleoprotein complexes that include a cas nuclease, an inhibitory nucleic acid, a chromatin stabilizing agent, or combinations thereof are animal models of a heart condition or heart disease.

The subjects can have fibroblasts exhibiting increased chromosomal accessibility in a Meox1 regulatory element, such as an enhancer. For example, the Meox1 regulatory element can be a peak 9/10 enhancer, such as the Meox1 enhancer on human chromosome 17 between about positions 43,589,381 and 43,595,263.

The subjects (e.g., patients, animals, and/or the animal model) can have a heart disease or heart condition. Such heart conditions or heart diseases can include cardiac fibrosis, lung fibrosis, kidney fibrosis, liver fibrosis, heart failure, congestive heart failure, myocardial infarction, cardiac ischemia, myocarditis, arrhythmia cardiomyopathy, dilated cardiomyopathy, cardiac artery disease, hypertension, valvular heart disease, hypertrophic cardiomyopathy (HCM), familial dilated cardiomyopathy (FDCM), restrictive cardiomyopathy (RCM), arrhythmogenic cardiomyopathy (AVC), unclassified cardiomyopathy, or a combination thereof. In some cases, the subject may not exhibit any symptoms of a heart disease or heart condition, in which case the test agent or therapeutic agent can be administered to inhibit the onset of a heart disease or a heart condition.

In some cases, knockout or knockdown of the Meox1 regulatory element can be used to modulate a subject's fibroblasts, myofibroblasts or a combination thereof. Such knockout or knockdown of the Meox1 regulatory element can be performed in vivo or in vitro within the cells or a subject. For example, knockout or knockdown of the Meox1 regulatory element can include CRISPR modification of a Meox1 regulatory element or use of a Meox1 inhibitory nucleic acid that targets a Meox1 regulatory element.

In vitro knockout or knockdown of the Meox1 regulatory element within a population of a subject's cells can be used to evaluate the patient's responses or to select a therapeutic agent for treatment of the subject. However, in some cases, in vitro knockout or knockdown of the Meox1 regulatory element within a population of a subject's cells can be used to generate modified cells, followed by reintroducing the modified fibroblasts to the patient. Such modified fibroblasts may not respond to stressful stimuli that would otherwise precipitate a cascade of problematic physiological responses that my result in fibrotic tissues. Hence, cardiac fibrosis, lung fibrosis, kidney fibrosis, liver fibrosis, and the related organ failure can be avoided by reduced Meox1 expression.

The modified cells can for example be modified fibroblasts, modified lung fibroblasts, modified myofibroblasts, modified cardiac fibroblasts, modified lung fibroblasts, modified liver fibroblasts, modified kidney fibroblasts, modified cardiac cells, or a combination thereof.

### Genetic Modulation

Described herein are guide RNAs that can modulate, knockdown or knockout Meox1 regulatory elements, including the Meox1 peak 9/10 enhancer element. The CRISPR-Cas9 genome-editing system can be used to delete modify Meox1 regulatory elements that are activated during heart conditions and diseases. A single guide RNA (sgRNA) can be used to recognize one or more target sequence in a subject's genome, and a nuclease can act as a pair of scissors to cleave a single-strand or a double-strand of genomic DNA. Mutations in the genome that are near the cleavage site can be introduced by an endogenous Non-Homologous End Joining (NHEJ) or Homology Directed Repair (HDR) pathway. Hence, the guide RNAs guide the nuclease to cleave the targeted Meox1 genomic site for deletion and/or modification by endogenous mechanisms.

The Meox1-specific guide RNAs can modify the Meox1 regulatory element so that it becomes less responsive to stress-activation that could induce signaling cascades that would trigger broad shifts in transcription and cell states that exacerbate pathologies.

The Cas system can recognize any sequence in the genome that matches 20 bases of a gRNA. However, each gRNA should also be adjacent to a “Protospacer Adjacent Motif” (PAM), which is invariant for each type of Cas protein, because the PAM binds directly to the Cas protein. See Doudna et al., Science 346(6213): 1077, 1258096 (2014); and Jinek et al., Science 337:816-21 (2012). Hence, the guide RNAs can have a PAM site sequence that can be bound by a Cas protein.

When the Cas system was first described for Cas9, with a “NGG” PAM site, the PAM was somewhat limiting in that it required a GG in the right orientation to the site to be targeted. Different Cas9 species have now been described with different PAM sites. See Jinek et al., Science 337:816-21 (2012); Ran et al., Nature 520:186-91 (2015); and Zetsche et al., Cell 163:759-71 (2015). In addition, mutations in the PAM recognition domain (Table 1) have increased the diversity of PAM sites for SpCas9 and SaCas9. See Kleinstiver et al., Nat Biotechnol 33:1293-1298 (2015); and Kleinstiver et al., Nature 523:481-5 (2015).

Table 1 summarizes information about PAM sites.

TABLE 1
PAM sites

| | PAM sites |
|---|---|
| SpCas9 | NGG |
| SpCas9 VRER variant | NGCG |
| SpCas9 EQR variant | NGAG |
| SpCas9 VQR variant | NGAN or NGNG |
| SaCas9 | NNGRRT |
| SaCas9, KKH variant | NNNRRT |
| FnCas2 (Cpf1) | TTN |
| DNA annotations: | |
| N = A, C, T or G | |
| R = Purine, A or G | |

Note that the guide RNAs for SpCas9 and SaCas9 cover 20 bases in the 5′ direction of the PAM site, while for FnCas2 (Cpf1) the guide RNA covers 20 bases to 3′ of the PAM.

There are a number of different types of nucleases and systems that can be used for gene editing. The nuclease employed can in some cases be any DNA binding protein with nuclease activity. Examples of nuclease include Streptococcus pyogenes Cas (SpCas9) nucleases, Staphylococcus aureus Cas9 (SpCas9) nucleases, Francisella novicida Cas2 (FnCas2, also called dFnCpf1) nucleases, Zinc Finger Nucleases (ZFN), Meganuclease, Transcription activator-like effector nucleases (TALEN), Fok-I nucleases, any DNA binding protein with nuclease activity, any DNA binding protein bound to a nuclease, or any combinations thereof. However, the CRISPR-Cas systems are generally the most widely used. In some cases, the nuclease is therefore a Cas nuclease.

CRISPR-Cas systems are generally divided into two classes. The class 1 system contains types I, III and IV, and the class 2 system contains types IL, V, and VI. The class 1 CRISPR-Cas system uses a complex of several Cas proteins, whereas the class 2 system only uses a single Cas protein with multiple domains. The class 2 CRISPR-Cas system is usually preferable for gene-engineering applications because of its simplicity and ease of use.

A variety of Cas nucleases can be employed in the methods described herein. Three species that have been best characterized are provided as examples. The most commonly used Cas nuclease is a Streptococcus pyogenes Cas9, (SpCas9). More recently described forms of Cas include Staphylococcus aureus Cas9 (SaCas9) and Francisella novicida Cas2 (FnCas2, also called FnCpf1). Jinek et al., Science 337:816-21 (2012); Qi et al., Cell 152:1173-83 (2013); Ran et al., Nature 520-186-91 (2015); Zetsche et al., Cell 163:759-71 (2015).

One example of an amino acid sequence for Streptococcus pyogenes Cas9 (SpCas9) nuclease is provided below (SEQ ID NO:4).

| | |
|---|---|
| 1 | MDKKYSIGLD IGTNSVGWAV ITDEYKVPSK KFKVLGNTDR |
| 41 | HSIKKNLIGA LLFDSGETAE ATRLKRTARR RYTRRKNRIC |
| 81 | YLQEIFSNEM AKVDDSFFHR LEESFLVEED KKHERHPIFG |
| 121 | NIVDEVAYHE KYPTIYHLRK KLVDSTDKAD LRLIYLALAH |
| 161 | MIKFRGHFLI EGDLNPDNSD VDKLFIQLVQ TYNQLFEENP |
| 201 | INASGVDAKA ILSARLSKSR RLENLIAQLP GEKKNGLFGN |
| 241 | LIALSLGLTP NFKSNFDLAE DAKLQLSKDT YDDDLDNLLA |
| 281 | QIGDQYADLE LAAKNLSDAI LLSDILRVNI EITKAPLSAS |
| 321 | MIKRYDEHHQ DLTLLKALVR QQLPEKYKEI FFDQSKNGYA |
| 361 | GYIDGGASQE EFYKFIKPIL EKMDGTEELL VKLNREDLLR |
| 401 | KQRTFDNGSI PHQIHLGELH AILRRQEDFY PFLKDNREKI |
| 441 | EKILTFRIPY YVGPLARGNS RFAWMTRKSE ETITPWNFEE |
| 481 | VVDKGASAQS FIERMTNFDK NLPNEKVLPK HSLLYEYFTV |
| 521 | YNELTKVKYV TEGMRKPAFL SGEQKKAIVD LLFKTNRKVT |
| 561 | VKQLKEDYFK KIECFDSVEI SGVEDRFNAS LGTYHDLLKI |
| 601 | IKDKDFLDNE ENEDILEDIV LTLTLFEDRE MIEERLKTYA |
| 641 | HLFDDKVMKQ LKRRRYTGWG RLSRKLINGI RDKQSGKTIL |
| 681 | DFLKSDGFAN RNFMQLIHDD SLTFKEDIQK AQVSGQGDSL |
| 721 | HEHIANLAGS PAIKKGILQT VKVVDELVKV MGRHKPENIV |
| 761 | IEMARENQTT QKGQKNSRER MKRIEEGIKE LGSQILKEHP |
| 801 | VENTQLQNEK LYLYYLQNGR DMYVDQELDI NRLSDYDVDH |
| 841 | IVPQSFLKDD SIDNKVLTRS DKNRGKSDNV PSEEVVKKMK |
| 881 | NYWRQLLNAK LITQRKFDNL TKAERGGLSE LDKAGFIKRQ |
| 921 | LVETRQITKH VAQILDSRMN TKYDENDKLI REVKVITLKS |
| 961 | KLVSDFRKDF QFYKVREINN YHHAHDAYLN AVVGTALIKK |
| 1001 | YPKLESEFVY GDYKVYDVRK MIAKSEQEIG KATAKYFFYS |
| 1041 | NIMNFFKTEI TLANGEIRKR PLIETNGETG EIVWDKGRDF |
| 1081 | ATVRKVLSMP QVNIVKKTEV QTGGFSKESI LPKRNSDKLI |
| 1121 | ARKKDWDPKK YGGFDSPTVA YSVLVVAKVE KGKSKKLKSV |
| 1161 | KELLGITIME RSSFEKNPID FLEAKGYKEV KKDLIIKLPK |
| 1201 | YSLFELENGR KRMLASAGEL QKGNELALPS KYVNFLYLAS |
| 1241 | HYEKLKGSPE DNEQKQLFVE QHKHYLDEII EQISEFSKRV |
| 1281 | ILADANLDKV LSAYNKHRDK PIREQAENII HLFTLTNLGA |
| 1321 | PAAFKYFDTT IDRKRYTSTK EVLDATLIHQ SITGLYETRI |
| 1361 | DLSQLGGD |

A cDNA that encodes the Streptococcus pyogenes Cas9 (SpCas9) is provided below (SEQ ID NO: 15).

```
      1 GACAAGAAGT ACAGCATCGG CCTGGACATC GGCACCAACT
    41 CTGTGGGCTG GGCCGTGATC ACCGACGAGT ACAAGGTGCC
    81 CAGCAAGAAA TTCAAGGTGC TGGGCAACAC CGACCGGCAC
  121 AGCATCAAGA AGAACCTGAT CGGAGCCCTG CTGTTCGACA
  161 GCGGCGAAAC AGCCGAGGCC ACCCGGCTGA AGAGAACCGC
  201 CAGAAGAAGA TACACCAGAC GGAAGAACCG GATCTGCTAT
  241 CTGCAAGAGA TCTTCAGCAA CGAGATGGCC AAGGTGGACG
  281 ACAGCTTCTT CCACAGACTG GAAGAGTCCT TCCTGGTGGA
  321 AGAGGATAAG AAGCACGAGC GGCACCCCAT CTTCGGCAAC
  361 ATCGTGGACG AGGTGGCCTA CCACGAGAAG TACCCCACCA
  401 TCTACCACCT GAGAAAGAAA CTGGTGGACA GCACCGACAA
  441 GGCCGACCTG CGGCTGATCT ATCTGGCCCT GGCCCACATG
  481 ATCAAGTTCC GGGGCCACTT CCTGATCGAG GGCGACCTGA
  521 ACCCCGACAA CAGCGACGTG GACAAGCTGT TCATCCAGCT
  561 GGTGCAGACC TACAACCAGC TGTTCGAGGA AAACCCCATC
  601 AACGCCAGCG GCGTGGACGC CAAGGCCATC CTGTCTGCCA
  641 GACTGAGCAA GAGCAGACGG CTGGAAAATC TGATCGCCCA
  681 GCTGCCCGGC GAGAAGAAGA ATGGCCTGTT CGGAAACCTG
  721 ATTGCCCTGA GCCTGGGCCT GACCCCCAAC TTCAAGAGCA
  761 ACTTCGACCT GGCCGAGGAT GCCAAACTGC AGCTGAGCAA
  801 GGACACCTAC GACGACGACC TGGACAACCT GCTGGCCCAG
  841 ATCGGCGACC AGTACGCCGA CCTGTTTCTG GCCGCCAAGA
  881 ACCTGTCCGA CGCCATCCTG CTGAGCGACA TCCTGAGAGT
  921 GAACACCGAG ATCACCAAGG CCCCCCTGAG CGCCTCTATG
  961 ATCAAGAGAT ACGACGAGCA CCACCAGGAC CTGACCCTGC
1001 TGAAAGCTCT CGTGCGGCAG CAGCTGCCTG AGAAGTACAA
1041 AGAGATTTTC TTCGACCAGA GCAAGAACGG CTACGCCGGC
1081 TACATTGACG GCGGAGCCAG CCAGGAAGAG TTCTACAAGT
1121 TCATCAAGCC CATCCTGGAA AAGATGGACG GCACCGAGGA
1161 ACTGCTCGTG AAGCTGAACA GAGAGGACCT GCTGCGGAAG
1201 CAGCGGACCT TCGACAACGG CAGCATCCCC CACCAGATCC
1241 ACCTGGGAGA GCTGCACGCC ATTCTGCGGC GGCAGGAAGA
1281 TTTTTACCCA TTCCTGAAGG ACAACCGGGA AAAGATCGAG
1321 AAGATCCTGA CCTTCCGCAT CCCCTACTAC GTGGGCCCTC
1361 TGGCCAGGGG AAACAGCAGA TTCGCCTGGA TGACCAGAAA
1401 GAGCGAGGAA ACCATCACCC CCTGGAACTT CGAGGAAGTG
1441 GTGGACAAGG GCGCTTCCGC CCAGAGCTTC ATCGAGCGGA
1481 TGACCAAGTT CGATAAGAAC CTGCCCAACG AGAAGGTGCT
1521 GCCCAAGCAC AGCCTGCTGT ACGAGTACTT CACCGTGTAT
1561 AACGAGCTGA CCAAAGTGAA ATACGTGACC GAGGGAATGA
1601 GAAAGCCCGC CTTCCTGAGC GGCGAGCAGA AAAAGGCCAT
1641 CGTGGACCTG CTGTTCAAGA CCAACCGGAA AGTGACCGTG
1681 AAGCAGCTGA AAGAGGACTA CTTCAAGAAA ATCGAGTGCT
1721 TCGACTCCGT GGAAATCTCC GGCGTGGAAG ATCGGTTCAA
1761 CGCCTCCCTG GGCACATACC ACGATCTGCT GAAAATTATC
1801 AAGGACAAGG ACTTCCTGGA CAATGAGGAA AACGAGGACA
1841 TTCTGGAAGA TATCGTGCTG ACCCTGACAC TGTTTGAGGA
1881 CAGAGAGATG ATCGAGGAAC GGCTGAAAAC CTATGCCCAC
1921 CTGTTCGACG ACAAAGTGAT GAAGCAGCTG AAGCGGCGGA
1961 GATACACCGG CTGGGGCAGG CTGAGCCGGA AGCTGATCAA
2001 CGGCATCCGG GACAAGCAGT CCGGCAAGAC AATCCTGGAT
2041 TTCCTGAAGT CCGACGGCTT CGCCAACAGA AACTTCATGC
2081 AGCTGATCCA CGACGACAGO CTGACCTTTA AAGAGGACAT
2121 CCAGAAAGCC CAGGTGTCCG GCCAGGGCGA TAGCCTGCAC
2161 GAGCACATTG CCAATCTGGC CGGCAGCCCC GCCATTAAGA
2201 AGGGCATCCT GCAGACAGTG AAGGTGGTGG ACGAGCTCGT
2241 GAAAGTGATG GGCCGGCACA AGCCCGAGAA CATCGTGATC
2281 GAAATGGCCA GAGAGAACCA GACCACCCAG AAGGGACAGA
2321 AGAACAGCCG CGAGAGAATG AAGCGGATCG AAGAGGGCAT
2361 CAAAGAGCTG GGCAGCCAGA TCCTGAAAGA ACACCCCGTG
2401 GAAAACACCC AGCTGCAGAA CGAGAAGCTG TACCTGTACT
2441 ACCTGCAGAA TGGGCGGGAT ATGTACGTGG ACCAGGAACT
2481 GGACATCAAC CGGCTGTCCG ACTACGATGT GGACCATATC
2521 GTGCCTCAGA GCTTTCTGAA GGACGACTCC ATCGACAACA
2561 AGGTGCTGAC CAGAAGCGAC AAGAACCGGG GCAAGAGCGA
2601 CAACGTGCCC TCCGAAGAGG TCGTGAAGAA GATGAAGAAC
2641 TACTGGCGGC AGCTGCTGAA CGCCAAGCTG ATTACCCAGA
2681 GAAAGTTCGA CAATCTGACC AAGGCCGAGA GAGGCGGCCT
2721 GAGCGAACTG GATAAGGCCG GCTTCATCAA GAGACAGCTG
2761 GTGGAAACCC GGCAGATCAC AAAGCACGTG GCACAGATCC
2801 TGGACTCCCG GATGAACACT AAGTACGACG AGAATGACAA
2841 GCTGATCCGG GAAGTGAAAG TGATCACCCT GAAGTCCAAG
2881 CTGGTGTCCG ATTTCCGGAA GGATTTCCAG TTTTACAAAG
2921 TGCGCGAGAT CAACAACTAC CACCACGCCC ACGACGCCTA
2961 CCTGAACGCC GTCGTGGGAA CCGCCCTGAT CAAAAAGTAC
3001 CCTAAGCTGG AAAGCGAGTT CGTGTACGGC GACTACAAGG
3041 TGTACGACGT GCGGAAGATG ATCGCCAAGA GCGAGCAGGA
3081 AATCGGCAAG GCTACCGCCA AGTACTTCTT CTACAGCAAC
3121 ATCATGAACT TTTTCAAGAC CGAGATTACC CTGGCCAACG
3161 GCGAGATCCG GAAGCGGCCT CTGATCGAGA CAAACGGCGA
3201 AACCGGGGAG ATCGTGTGGG ATAAGGGCCG GGATTTTGCC
3241 ACCGTGCGGA AAGTGCTGAG CATGCCCCAA GTGAATATCG
3281 TGAAAAAGAC CGAGGTGCAG ACAGGCGGCT TCAGCAAAGA
3321 GTCTATCCTG CCCAAGAGGA ACAGCGATAA GCTGATCGCC
3361 AGAAAGAAGG ACTGGGACCC TAAGAAGTAG GGCGGCTTCG
3401 ACAGCCCCAC CGTGGCCTAT TCTGTGCTGG TGGTGGCCAA
3441 AGTGGAAAAG GGCAAGTCCA AGAAACTGAA GAGTGTGAAA
3481 GAGCTGCTGG GGATCACCAT CATGGAAAGA AGCAGCTTCG
3521 AGAAGAATCC CATCGACTTT CTGGAAGCCA AGGGCTACAA
3561 AGAAGTGAAA AAGGACCTGA TCATCAAGCT GCCTAAGTAC
3601 TCCCTGTTCG AGCTGGAAAA CGGCCGGAAG AGAATGCTGG
3641 CCTCTGCCGG CGAACTGCAG AAGGGAAACG AACTGGCCCT
3681 GCCCTCCAAA TATGTGAACT TCCTGTACCT GGCCAGCCAC
3721 TATGAGAAGC TGAAGGGCTC CCCCGAGGAT AATGAGCAGA
3761 AACAGCTGTT TGTGGAACAG CACAAGCACT ACCTGGACGA
3801 GATCATCGAG CAGATCAGCG AGTTCTCCAA GAGAGTGATC
3841 CTGGCCGACG CTAATCTGGA CAAAGTGCTG TGCGCCTACA
3881 ACAAGCACCG GGATAAGCCC ATCAGAGAGC AGGCCGAGAA
3921 TATCATCCAC CTGTTTACCC TGACCAATCT GGGAGCCCCT
3961 GCCGCCTTCA AGTACTTTGA CACCACCATC GACCGGAAGA
4001 GGTACACCAG CACCAAAGAG GTGCTGGACG CCACCCTGAT
4041 CCACCAGAGC ATCACCGGCC TGTAGGAGAC ACGGATCGAC
4081 CTGTCTCAGC TGGGAGGCGA C
```

An amino acid sequence for a Francisella novicida Cas2 (FnCas2, also called FnCpf1) is shown below (SEQ ID NO:16).

| | |
|---|---|
| 1 | MTQFEGFTNL YQVSKTLRFE LTPQGKTLKH IQEQGFTEED |
| 41 | KARNDHYKEL KPIIDRIYKT YADQCLQLVQ LDWENLSAAI |
| 81 | DSYRKEKTEE TRNALIEEQA TYRNAIHDYF IGRTDNLTDA |
| 121 | INKRHAEIYK GLFKAELFNG KVLKQLGTVT TTEHENALLR |
| 161 | SFDKFTTYFS GFYENRKNVF SAEDISTAIP HRIVQDNFPK |
| 201 | FRENCHTFTR LITAVPSLRE HFENVKKAIG TFVSTSIEEV |
| 241 | FSFPFYNQLL TQTQIDLYNQ LLGGISREAG TEKIKGLNEV |
| 281 | LNLAIQKNDE TAHIIASLPH RFIPLFKQIL SDRNTLSFTL |
| 321 | EEFKSDEEVI QSFCKYKTLL RNENVLETAE ALFNELNSID |
| 361 | LTHIFISHKK LETISSALCD HWDTLRNALY ERRISELTGK |
| 401 | ITKSAKEKVQ RSLKHEDINL QEIISAAGKE LSEAFKQKTS |
| 441 | EILSHAHAAL DQPLPTTLKK QEEKEILKSQ LDSLLGLYHL |
| 481 | LDWFAVDESN EVDPEFSARL TGIKLEMEPS LSFYNKARNY |
| 521 | ATKKPYSVEK FKLNFQMPTL ASGWDVNKEK NNGAILFVKN |
| 561 | GLYYLGIMPK QKGRYKALSF EPTEKTSEGF DKMYYDYFPD |
| 601 | AAKMIPKCST QLKAVTAHFQ THTTPILLSN NFIEPLEITK |
| 641 | EIYDLNNPEK EPKKFQTAYA KKTGDQKGYR EALCKWIDFT |
| 681 | RDFLSKYTKT TSIDLSSLRP SSQYKDLGEY YAELNPLLYH |
| 721 | ISFQRIAEKE IMDAVETGKL YLFQIYNKDF AKGHHGKPNL |
| 761 | HTLYWTGLFS PENLAKTSIK LNGQAELFYR PKSRMKRMAH |
| 801 | RLGEKMLNKK LKDQKTPIPD TLYQELYDYV NHRLSHDLSD |
| 841 | EARALLPNVI TKEVSHEIIK DRRFTSDKFF FHVPTTLNYQ |
| 881 | AANSPSKFNQ RVNAYLKEHP ETPIIGIDRG ERNLIYITVI |
| 921 | DSTGKILEQR SLNTIQQFDY QKKLDNREKE RVAARQAWSV |
| 961 | VGTIKDLKQG YLSQVIHEIV DLMIHYQAVV VLENLNFGFK |
| 1001 | SKRTGIAEKA VYQQFEKMLI DKLNCLVLKD YPAEKVGGVL |
| 1041 | NPYQLTDQFT SFAKMGTQSG FLFYVPAPYT SKIDPLTGFV |
| 1081 | DPFVWKTIKN HESRKHFLEG FDFLHYDVKT GDFILHFKMN |
| 1121 | RNLSFQRGLP GFMPAWDIVE EKNETQFDAK GTPFIAGKRI |
| 1161 | VPVIENHRFT GRYRDLYPAN ELIALLEEKG IVFRDGSNIL |
| 1201 | PKLLENDDSH AIDTMVALIR SVLQMRNSNA ATGEDYINSP |
| 1241 | VRDLNGVCFD SRFQNPEWPM DADANGAYHI ALKGQLLLNH |
| 1281 | LKESKDLKLQ NGISNQDWLA YIQELRN |

A cDNA that encodes the foregoing Francisella novicida Cas2 (FnCas2, also called FnCpf1) polypeptide is shown below (SEQ ID NO: 17).

```
      1 ATGACACAGT TCGAGGGCTT TACCAACCTG TATCAGGTGA
    41 GCAAGACACT GCGGTTTGAG CTGATCCCAC AGGGCAAGAC
    81 CCTGAAGCAC ATCCAGGAGC AGGGCTTCAT CGAGGAGGAC
  121 AAGGCCCGCA ATGATCACTA CAAGGAGCTG AAGCCCATCA
  161 TCGATCGGAT CTACAAGACC TATGCCGACC AGTGCCTGCA
  201 GCTGGTGCAG CTGGATTGGG AGAACCTGAG CGCCGCCATC
  241 GACTCCTATA GAAAGGAGAA AACCGAGGAG ACAAGGAACG
  281 CCCTGATCGA GGAGCAGGCC ACATATCGCA ATGCCATCCA
  321 CGACTACTTC ATCGGCCGGA CAGACAACCT GACCGATGCC
  361 ATCAATAAGA GACACGCCGA GATCTACAAG GGCCTGTTCA
  401 AGGCCGAGCT GTTTAATGGC AAGGTGCTGA AGCAGCTGGG
  441 CACCGTGACC ACAACCGAGC ACGAGAACGC CCTGCTGCGG
  481 AGCTTCGACA AGTTTACAAC CTACTTCTCC GGCTTTTATG
  521 AGAACAGGAA GAACGTGTTC AGCGCCGAGG ATATCAGCAC
  561 AGCCATCCCA CACCGCATCG TGCAGGACAA CTTCCCCAAG
  601 TTTAAGGAGA ATTGTCACAT CTTCACACGC CTGATCACCG
  721 CCGTGCCCAG CCTGCGGGAG CACTTTGAGA ACGTGAAGAA
  761 GGCCATCGGC ATCTTCGTGA GCACCTCCAT CGAGGAGGTG
  801 TTTTCCTTCC CTTTTTATAA CCAGCTGCTG ACACAGACCC
  841 AGATCGACCT GTATAACCAG CTGCTGGGAG GAATCTCTCG
  881 GGAGGCAGGC ACCGAGAAGA TCAAGGGCCT GAACGAGGTG
  921 CTGAATCTGG CCATCCAGAA GAATGATGAG ACAGCCCACA
  961 TCATCGCCTC CCTGCCACAC AGATTCATCC CCCTGTTTAA
1001 GCAGATCCTG TCCGATAGGA ACACCCTGTC TTTCATCCTG
1041 GAGGAGTTTA AGAGCGACGA GGAAGTGATC CAGTCCTTCT
1081 GCAAGTACAA GACACTGCTG AGAAACGAGA ACGTGCTGGA
1121 GACAGCCGAG GCCCTGTTTA ACGAGCTGAA CAGCATCGAC
1161 CTGACACACA TCTTCATCAG CCACAAGAAG CTGGAGACAA
1201 TCAGCAGCGC CCTGTGCGAC CACTGGGATA CACTGAGGAA
1241 TGCCCTGTAT GAGCGGAGAA TCTCCGAGCT GACAGGCAAG
1281 ATCACCAAGT CTGCCAAGGA GAAGGTGCAG CGCAGCCTGA
1321 AGCACGAGGA TATCAACCTG CAGGAGATCA TCTCTGCCGC
1361 AGGCAAGGAG CTGAGCGAGG CCTTCAAGCA GAAAACCAGC
1401 GAGATCCTGT CCCACGCACA CGCCGCCCTG GATCAGCCAC
1441 TGCCTACAAC CCTGAAGAAG CAGGAGGAGA AGGAGATCCT
1481 GAAGTCTCAG CTGGACAGCC TGCTGGGCCT GTACCACCTG
1521 CTGGACTGGT TTGCCGTGGA TGAGTCCAAC GAGGTGGACC
1561 CCGAGTTCTC TGCCCGGCTG ACCGGCATCA AGCTGGAGAT
1601 GGAGCCTTCT CTGAGCTTCT ACAACAAGGC CAGAAATTAT
1641 GCCACCAAGA AGCCCTACTC CGTGGAGAAG TTCAAGCTGA
1681 ACTTTCAGAT GCCTACACTG GCCTCTGGCT GGGACGTGAA
1721 TAAGGAGAAG AACAATGGCG CCATCCTGTT TGTGAAGAAC
1761 GGCCTGTACT ATCTGGGCAT CATGCCAAAG CAGAAGGGCA
1801 GGTATAAGGC CCTGAGCTTC GAGCCCACAG AGAAAACCAG
1841 CGAGGGCTTT GATAAGATGT ACTATGACTA CTTCCCTGAT
1881 GCCGCCAAGA TGATCCCAAA GTGCAGCACC CAGCTGAAGG
1921 CCGTGACAGC CCACTTTCAG ACCCACACAA CCCCCATCCT
1961 GCTGTCCAAC AATTTCATCG AGCCTCTGGA GATCACAAAG
2001 GAGATCTAGG ACCTGAACAA TCCTGAGAAG GAGCCAAAGA
2041 AGTTTCAGAC AGCCTACGCC AAGAAAACCG GCGACCAGAA
2081 GGGCTACAGA GAGGCCCTGT GCAAGTGGAT CGACTTCACA
2121 AGGGATTTTC TGTCCAAGTA TACCAAGACA ACCTGTATCG
2161 ATCTGTCTAG CCTGCGGCCA TCCTCTCAGT ATAAGGACCT
2201 GGGCGAGTAC TATGCCGAGC TGAATCCCCT GCTGTACCAC
2241 ATCAGCTTCC AGAGAATCGC CGAGAAGGAG ATCATGGATG
2281 CCGTGGAGAC AGGCAAGCTG TACCTGTTCC AGATCTATAA
2321 CAAGGACTTT GCCAAGGGCC ACCACGGCAA GCCTAATCTG
2361 CACACACTGT ATTGGACCGG CCTGTTTTCT CCAGAGAACC
2401 TGGCCAAGAC AAGCATCAAG CTGAATGGCC AGGCCGAGCT
2441 GTTCTACCGC CCTAAGTCCA GGATGAAGAG GATGGCACAC
2481 CGGCTGGGAG AGAAGATGCT GAACAAGAAG CTGAAGGATC
2521 AGAAAACCCC AATCCCCGAC ACCCTGTACC AGGAGCTGTA
2561 CGACTATGTG AATCACAGAC TGTCCCACGA CCTGTCTGAT
2601 GAGGCCAGGG CCCTGCTGCC CAACGTGATC ACCAAGGAGG
2641 TGTCTCACGA GATCATCAAG GATAGGCGCT TTACCAGCGA
2681 CAAGTTCTTT TTCCACGTGC CTATCACACT GAACTATCAG
2721 GCCGCCAATT CCCCATCTAA GTTCAACCAG AGGGTGAATG
2761 CCTACCTGAA GGAGCACCCC GAGACACCTA TCATCGGCAT
2801 CGATCGGGGC GAGAGAAACC TGATCTATAT CACAGTGATC
2841 GCCTCCACCG GCAAGATCCT GGAGCAGCGG AGCCTGAACA
2881 CCATCCAGCA GTTTGATTAC CAGAAGAAGC TGGACAACAG
2921 GGAGAAGGAG AGGGTGGCAG CAAGGCAGGC CTGGTCTGTG
2961 GTGGGCACAA TCAAGGATCT GAAGCAGGGC TATCTGAGCC
3001 AGGTCATCCA CGAGATCGTG GACCTGATGA TCCACTACCA
3041 GGCCGTGGTG GTGCTGGAGA ACCTGAATTT CGGCTTTAAG
3081 AGCAAGAGGA CCGGCATCGC CGCGAAGGCC GTGTACCAGC
3121 AGTTCGAGAA GATGCTGATC GATAAGCTGA ATTGCCTGGT
3161 GCTGAAGGAC TATCCAGCAG AGAAAGTGGG AGGCGTGCTG
3201 AACCCATACC AGCTGACAGA CCAGTTCACC TCCTTTGCCA
3241 AGATGGGCAC CCAGTCTGGC TTCCTGTTTT ACGTGCCTGC
3281 CCCATATACA TCTAAGATCG ATCCCCTGAC CGGCTTCGTG
3321 GACCCCTTCG TGTGGAAAAC CATCAAGAAT CACGAGAGCC
3361 GCAAGCACTT CCTGGAGGGC TTCGACTTTC TGCACTACGA
3401 CGTGAAAACC GGCGACTTCA TCCTGCACTT TAAGATGAAC
3441 AGAAATCTGT CCTTCCAGAG GGGCCTGCCC GGCTTTATGC
3481 CTGCATGGGA TATCGTGTTC GAGAAGAACG AGACACAGTT
3521 TGACGCCAAG GGCACCCCTT TCATCGCCGG CAAGAGAATC
3561 GTGCCAGTGA TCGAGAATCA CAGATTCACC GGCAGATACC
3601 GGGACCTGTA TCCTGCCAAC GAGCTGATCG CCCTGCTGGA
3641 GGAGAAGGGC ATCGTGTTCA GGGATGGCTC CAACATCCTG
3681 CCAAAGCTGC TGGAGAATGA CGATTCTCAC GCCATCGACA
3721 CCATGGTGGC CCTGATCCGC AGCGTGCTGC AGATGCGGAA
3761 CTCCAATGCC GCCACAGGCG AGGACTATAT CAACAGCCCC
3801 GTGCGCGATC TGAATGGCGT GTGCTTCGAC TCCCGGTTTC
3841 AGAACCCAGA GTGGCCCATG GACGCCGATG CCAATGGCGC
3881 CTACCACATC GCCCTGAAGG GCCAGCTGCT GCTGAATCAC
3921 CTGAAGGAGA GCAAGGATCT GAAGCTGCAG AACGGCATCT
3961 CCAATCAGGA CTGGCTGGCC TACATCCAGG AGCTGCGCAA
4001 C
```

Nucleic Acids that Inhibit Meox1

As described herein, reduction in Meox1 expression can improve cardiac function. Moreover, the data provided herein shows that the peak 9/10 enhance is transcribed in Meox1 nascent transcripts.

Inhibitory nucleic acids can be used to reduce the expression and/or translation of Meox1. Such inhibitory nucleic acids can specifically bind to Meox1 nucleic acids, including nascent RNAs, that encode Meox1 and/or an Meox1 enhancer (e.g., the peak 9/10 Meox1 enhancer element). Anti-sense oligonucleotides have been used to silence other enhancers, including enhancers that can regulate cardiac fibroblast proliferation, migration, and survival (see, e.g., Micheletti et al. Sci. Transl. Med. 9 (395) eaai9l 18 (2017)). Hence, even though an enhancer may be distance from an amino acid coding region, an enhancer can still be silenced by inhibitory nucleic acids.

An inhibitory nucleic acid can have at least one segment that will hybridize to Meox1 nucleic acid under intracellular or stringent conditions. The inhibitory nucleic acid can reduce processing, expression, and/or translation of a nucleic acid encoding Meox1. An inhibitory nucleic acid may hybridize to a genomic DNA, a messenger RNA, nascent RNA, or a combination thereof. An inhibitory nucleic acid may be incorporated into a plasmid vector or viral DNA. It may be single stranded or double stranded, circular, or linear.

An inhibitory nucleic acid can be a polymer of ribose nucleotides (RNAi) or deoxyribose nucleotides having more than 13 nucleotides in length. An inhibitory nucleic acid may include naturally occurring nucleotides; synthetic, modified, or pseudo-nucleotides such as phosphorothiolates; as well as nucleotides having a detectable label such as P³², biotin or digoxigenin. An inhibitory nucleic acid can reduce the expression, processing, and/or translation of a Meox1 nucleic acid. Such an inhibitory nucleic acid may be completely complementary to a segment of Meox1 nucleic acid (e.g., a Meox1 mRNA or Meox1 nascent transcript that includes at least one Meox1 enhancer element such as the peak 9/10 enhancer).

An inhibitory nucleic acid can hybridize to a Meox1 nucleic acid under intracellular conditions or under stringent hybridization conditions and is sufficient to inhibit expression of a Meox1 nucleic acid. Intracellular conditions refer to conditions such as temperature, pH and salt concentrations typically found inside a cell, e.g. a target cell described herein.

Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to a Meox1 coding or flanking sequence, can each be separated by a stretch of contiguous nucleotides that are not complementary to adjacent coding sequences, and such an inhibitory nucleic acid can still inhibit the function of a Meox1 nucleic acid. In general, each stretch of contiguous nucleotides is at least 4, 5, 6, 7, or 8 or more nucleotides in length. Non-complementary intervening sequences may be 1, 2, 3, or 4 nucleotides in length.

One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme, or an antisense nucleic acid molecule.

The inhibitory nucleic acid molecule may be single (e.g., an antisense oligonucleotide) or double stranded (e.g., a siRNA) and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex. Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-O alkyl (usually in chimeras with RNase-H dependent antisense), peptide nucleic acid (PNA), locked nucleic acid (LNA) and morpholino antisense.

Small interfering RNAs (siRNAs), for example, may be used to specifically reduce Meox1 processing or translation such that production of the encoded polypeptide is reduced. SiRNAs mediate post-transcriptional gene silencing in a sequence-specific manner. See, for example, website at invitrogen.com/site/us/en/home/Products-and-Services/Applications/rnai.html. Once incorporated into an RNA-induced silencing complex, siRNA can mediate cleavage of the homologous endogenous mRNA transcript by guiding the complex to the homologous mRNA transcript, which is then cleaved by the complex. The siRNA may be homologous to any region of the Meorl mRNA transcript. The region of homology may be 50 nucleotides or less, 30 nucleotides or less in length, such as less than 25 nucleotides, or for example about 21 to 23 nucleotides in length. SiRNA is typically double stranded and may have two-nucleotide 3′ overhangs, for example, 3′ overhanging UU dinucleotides. Methods for designing siRNAs are available, see, for example, Elbashir et al. Nature 411: 494-498 (2001); Harborth et al. Antisense Nucleic Acid Drug Dev. 13: 83-106 (2003).

The pSuppressorNeo vector for expressing hairpin siRNA, commercially available from IMGENEX (San Diego, Calif.), can be used to make siRNA or shRNA for inhibiting Meox1 expression. The construction of the siRNA or shRNA expression plasmid involves the selection of the target region of the mRNA, which can be a trial-and-error process. However, Elbashir et al. have provided guidelines that appear to work ˜80% of the time. Elbashir, S. M., et al., Analysis of gene function in somatic mammalian cells using small interfering RNAs. Methods, 2002. 26(2): p. 199-213. Accordingly, for synthesis of synthetic siRNA or shRNA, a target region may be selected preferably 50 to 100 nucleotides downstream of the start codon. The 5′ and 3′ untranslated regions and regions close to the start codon should be avoided as these may be richer in regulatory protein binding sites. As siRNA can begin with AA, have 3′ UU overhangs for both the sense and antisense siRNA strands, and have an approximate 50% G/C content. An example of a sequence for a synthetic siRNA or shRNA is 5′-AA(N19)UU, where N is any nucleotide in the mRNA sequence and should be approximately 50% G-C content. The selected sequence(s) can be compared to others in the human genome database to minimize homology to other known coding sequences (e.g., by Blast search, for example, through the NCBI website).

Inhibitory nucleic acids (e.g., siRNAs, and/or anti-sense oligonucleotides) may be chemically synthesized, created by in vitro transcription, or expressed from an expression vector or a PCR expression cassette. See, e.g., website at invitrogen.com/site/us/en/home/Products-and-Services/Applications/rnai.html.

When an siRNA is expressed from an expression vector or a PCR expression cassette, the insert encoding the siRNA may be expressed as an RNA transcript that folds into an siRNA hairpin or a shRNA. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, or about 3 to 23 nucleotides in length, and may include various nucleotide sequences including for example, AUG, CCC, UUCG, CCACC, CTCGAG, AAGCUU, and CCACACC. SiRNAs also may be produced in vivo by cleavage of double-stranded RNA introduced directly or via a transgene or virus. Amplification by an RNA-dependent RNA polymerase may occur in some organisms.

An inhibitory nucleic acid such as a short hairpin RNA siRNA or an antisense oligonucleotide may be prepared using methods such as by expression from an expression vector or expression cassette that includes the sequence of the inhibitory nucleic acid. Alternatively, it may be prepared by chemical synthesis using naturally-occurring nucleotides, modified nucleotides, or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target Meox1 nucleic acid.

### Delivery

There are different ways to deliver inhibitory nucleic acids, guide RNAs, nucleases, or combinations thereof. In some cases, the inhibitory nucleic acids, guide RNAs, nucleases, or combinations thereof are directly administered to a subject. In other cases, the inhibitory nucleic acids, guide RNAs, nucleases, or combinations thereof can be encoded in one or more expression cassettes or expression vectors, and the expression cassettes/vectors can be administered to a subject. Hence, the inhibitory nucleic acids, guide RNAs, nucleases, or combinations thereof can be expressed in vivo from expression cassettes/expression vectors.

The first and probably the most straightforward approach is to use a vector-based CRISPR-Cas9 system encoding the nuclease and guide RNA (e.g., sgRNA) from the same vector, thus avoiding multiple transfections of different components. The second is to deliver the mixture of the Cas9 mRNA and the sgRNA, and the third strategy is to deliver the mixture of the Cas9 protein and the sgRNA.

In some cases, the guide RNAs can be delivered to cells or administered to subjects in the form of an expression cassette or vector that can express one or more of the guide RNAs. Nucleases can also be delivered to cells or administered to the subjects in the form of an expression cassette or vector that can express one or more nucleases. The nucleases can also be combined with their respective gRNAs and delivered as RNA-protein complexes (RNPs). Hence, the RNPs can be pre-assembled outside of the cell and introduced into the cell.

Inhibitory nucleic acids, guide RNAs can be expressed from expression cassettes or vectors. A nuclease can also be expressed in the same cell with one or more gRNAs. The inhibitory nucleic acids, guide RNAs and nucleases can be introduced in form of a nucleic acid molecules encoding the inhibitory nucleic acids, guide RNAs and/or nucleases. Such nucleic acid molecules can be provided in expression cassettes or expression vectors.

The expression cassettes can be within vectors. Vectors can, for example, be expression vectors such as viruses or other vectors that is readily taken up by the cells. Examples of vectors that can be used include, for example, adeno-associated virus (AAV) gene transfer vectors, lentiviral vectors, retroviral vectors, herpes virus vectors, e.g., cytomegalovirus vectors, herpes simplex virus vectors, varicella zoster virus vectors, adenovirus vectors, e.g., helper-dependent adenovirus vectors, adenovirus-AAV hybrids, rabies virus vectors, vesicular stomatitis virus (VSV) vectors, coronavirus vectors, poxvirus vectors and the like. Non-viral vectors may be employed to deliver the expression vectors, e.g., liposomes, nanoparticles, microparticles, lipoplexes, polyplexes, nanotubes, and the like. In one embodiment, two or more expression vectors are administered, for instance, each encoding a distinct inhibitory nucleic acid, guide RNA, a distinct nuclease, or a combination thereof.

The expression cassettes or expression vectors include promoter sequences that are operably linked to the nucleic acid segment encoding the inhibitory nucleic acids, guide RNAs, nucleases, or combinations thereof. Methods for ensuring expression of a functional inhibitory nucleic acid, guide RNA, nuclease or combinations thereof can involve expression from a transgene, expression cassette, or expression vector. For example, the nucleic acid segments encoding the selected inhibitory nucleic acids, guide RNAs, nucleases, or combinations thereof can be present in a vector, such as for example a plasmid, cosmid, virus, bacteriophage, or another vector available for genetic engineering. The coding sequences inserted in the vector can be synthesized by standard methods or isolated from natural sources. The coding sequences may further be ligated to transcriptional regulatory elements, termination sequences, and/or to other amino acid encoding sequences. Such regulatory sequences can provide initiation of transcription, internal ribosomal entry sites (IRES) (Owens, Proc. Natl. Acad. Sci. USA 98: 1471-1476 (2001)) and optionally regulatory elements ensuring termination of transcription and stabilization of the transcript.

Non-limiting examples for regulatory elements ensuring the initiation of transcription comprise a translation initiation codon, transcriptional enhancers such as e.g. the SV40-enhancer, insulators and/or promoters. The promoter can be a constitutive promoter, and inducible promoter, or a tissue-specific promoter. Examples of promoters that can be used include the cytomegalovirus (CMV) promoter, SV40-promoter, RSV-promoter (Rous sarcoma virus), the lacZ promoter, chicken beta-actin promoter, CAG-promoter (a combination of chicken beta-actin promoter and cytomegalovirus immediate-early enhancer), the gai10 promoter, human elongation factor 1α-promoter, AOX1 promoter, GAL 1 promoter CaM-kinase promoter, the lac, trp or tac promoter, the lacUV5 promoter, the Autographa californica multiple nuclear polyhedrosis virus (AcMNPV) polyhedral promoter, or a globin intron in mammalian and other animal cells. Non-limiting examples for regulatory elements ensuring transcription termination include the V40-poly-A site, the tk-poly-A site, or the SV40, lacZ or AcMNPV polyhedral polyadenylation signals, which are to be included downstream of the nucleic acid sequence of the invention. Additional regulatory elements may include translational enhancers, Kozak sequences and intervening sequences flanked by donor and acceptor sites for RNA splicing. Moreover, elements such as origin of replication, drug resistance gene or regulators (as part of an inducible promoter) may also be included.

The expression cassettes and/or expression vectors can be introduced into cells. The cells can be any mammalian or avian cell. For example, the cells can be human cells, or cells from a domesticated animal, a zoo animal, or an experimental animal.

The cells can be obtained from a subject in need of treatment. The cells can be autologous or allogenic cells relative to a subject. In some cases, the cells can be fibroblasts, myofibroblasts, cardiac fibroblasts, induced pluripotent stem cells, cardiac progenitor cells, cardiomyocytes and/or cardiac cells. The allogenic cells can be typed to match those of a subject.

The guide RNAs can also be introduced into cells or administered to subjects in the form of RNA-protein complexes (RNPs). The nuclease can be pre-bound with one or more gRNAs prior to introduction into cells. The advantage RNP delivery of Cas-gRNA complexes is that complex formation it is readily controlled ex vivo and the selected Cas polypeptides can independently be complexed with selected guide RNAs so that the structure and compositions of the desired complexes is known with certainty. The RNPs are quite stable, with no apparent exchange of gRNAs. Hence, the nuclease-gRNA RNP can carry a selected gRNA to the site of genomic editing.

For example, Cas RNP can be prepared by incubating the Cas proteins with the selected gRNA using a molar excess of gRNA relative to protein (e.g., using about a 1:1.1 to 1:1.4 protein to gRNA molar ratio). The buffer to be used during such incubation can include 20 mM HEPES (pH 7.5), 150 mM KCl, 1 mM MgCl, 10% glycerol and 1 mM TCEP. Incubation can be done at 37° C. for about 5 minutes to about 30 minutes (usually 10 minutes is sufficient). When reference DNA or an HDR template is used, it can be added to the Cas RNP.

Nucleofection can be employed to introduce the Cas RNP into cells. See Lin et al., Enhanced homology-directed human genome engineering by controlled timing of CRISPR/Cas9 delivery. Elife 3:e04766. For example, nucleofection reactions can involve mixing approximately 1×10 μl×1×10-cells in about 10 μl to 40 μl of nucleofection reagent with about 5μ to 30 μl of RNP:DNA. In some instances, about 2×10, cells are mixed with about 20 sd of nucleofection reagent and about 10 μl RNP:DNA. After electroporation, growth media is added, and the cells are transferred to tissue culture plates for growth and evaluation. The nucleofection reagents and machines are available from Lonza (Allendale, N.J.).

Thus, the invention provides agents for use in medical therapy, such as gene therapy vectors that treat, inhibit, or prevent cardiac conditions and diseases.

### Administration

Guide RNAs, or expression cassettes/expression vectors that can express the guide RNA can be administered to subjects. Cells (e.g., fibroblasts) that have been modified reduce Meox1 transcription and/or Meox1 enhancer activation can also be administered to subjects. Such guide RNAs, expression cassettes, expression vectors, and cells generated as described herein can be employed for treatment or prevention of cardiac conditions and/or diseases in a human patient or other subjects. Patients or subjects can be in need of such treatment. In some cases, the patients or subjects may not yet exhibit any symptoms of a cardiac condition/disease or another medical condition.

The guide RNAs, expression cassettes, expression vectors, and cells are administered in a manner that permits them to be incorporated into, graft or migrate to a specific tissue site, such as into cardiac tissues. Such guide RNAs, expression cassettes, expression vectors, and cells can reconstitute or regenerate functionally deficient areas of tissues, including cardiac tissues. Devices are available that can be adapted for administering cells, for example, to cardiac tissues.

For therapy, guide RNAs, expression cassettes, expression vectors, and/or cells (e.g., fibroblasts, myofibroblasts, cardiac cells, and the like) can be administered locally or systemically. Administration can be by injection, catheter, implantable device, or the like. The guide RNAs, expression cassettes, expression vectors, and cells can be administered in any physiologically acceptable excipient or carrier that does not adversely affect the subject. For example, the guide RNAs, expression cassettes, expression vectors, and cells can be administered intravenously or through an intracardiac route (e.g., epicardially or intramyocardially). Methods of administering the guide RNAs, expression cassettes, expression vectors, and/or cells to subjects, particularly human subjects, include injection or implantation of the guide RNAs, expression cassettes, expression vectors, and cells into target sites or they can be inserted into a delivery device which facilitates introduction, uptake, incorporation, or implantation of the expression cassettes, expression vectors, and cells. Such delivery devices include tubes, e.g., catheters, for introducing cells, expression vectors, and fluids into the body of a recipient subject. The tubes can additionally include a needle, e.g., a syringe, through which the cells of the invention can be introduced into the subject at a desired location. Multiple injections may be made using this procedure.

As used herein, the term “solution” includes a carrier or diluent in which the guide RNAs, expression cassettes, expression vectors, and cells of the invention remain viable and/or functional. Carriers and diluents that can be used include saline, aqueous buffer solutions, solvents and/or dispersion media. The use of such carriers and diluents are available in the art. The solution is preferably sterile and fluid to the extent that easy syringability exists.

The guide RNAs, expression cassettes, expression vectors, and cells can also be embedded in a support matrix. Suitable ingredients include matrix proteins that support or promote the incorporation of adhesion of the guide RNAs, expression cassettes, expression vectors, and modified cells. In another embodiment, the composition may include physiologically acceptable matrix scaffolds. Such physiologically acceptable matrix scaffolds can be resorbable and/or biodegradable.

In some cases, cardiac cells can be modified to express the guide RNAs and optionally the nuclease. In addition, cardiac cells can be modified by the guide RNAs and nucleases to generate a population of modified cells that have reduced Meox1 expression and/or reduced activation of at least one Meox1 regulatory element.

A population of modified cells generated by the methods described herein can include low percentages of non-fibroblast cells (e.g., other cardiac cells and/or endothelial cells). For example, a population of modified cells for use in compositions and for administration to subjects can have less than about 90% non-fibroblast cells, less than about 85% non-fibroblast cells, less than about 80% non-fibroblast cells, less than about 75% non-fibroblast cells, less than about 70% non-fibroblast cells, less than about 65% non-fibroblast cells, less than about 60% non-fibroblast cells, less than about 55% non-fibroblast cells, less than about 50% non-fibroblast cells, less than about 45% non-fibroblast cells, less than about 40% non-fibroblast cells, less than about 35% non-fibroblast cells, less than about 30% non-fibroblast cells, less than about 25% non-fibroblast cells, less than about 20% non-fibroblast cells, less than about 15% non-fibroblast cells, less than about 12% non-fibroblast cells, less than about 10% non-fibroblast cells, less than about 8% non-fibroblast cells, less than about 6% non-fibroblast cells, less than about 5% non-fibroblast cells, less than about 4% non-fibroblast cells, less than about 3% non-fibroblast cells, less than about 2% non-fibroblast cells, or less than about 1% non-fibroblast cells of the total cells in the cell population.

Many cell types are capable of migrating to an appropriate site for regeneration and differentiation within a subject. To determine the suitability of various therapeutic administration regimens and dosages of cell compositions, the fibroblasts or other types of cells can first be tested in a suitable animal model. At one level, cells are assessed for their ability to survive and maintain their phenotype in vivo. Cells can also be assessed to ascertain whether they migrate to diseased or injured sites in vivo, or to determine an appropriate number, or dosage, of cells to be administered. Cell compositions can be administered to immunodeficient animals (such as nude mice, or animals rendered immunodeficient chemically or by irradiation). Tissues can be harvested after a period of regrowth and assessed as to whether the administered cells or progeny thereof are still present, are alive, and/or have migrated to desired or undesired locations.

Injected fibroblasts or other cell types can be traced by a variety of methods. For example, cells containing or expressing a detectable label (such as green fluorescent protein, or beta-galactosidase) can readily be detected. The cells can be pre-labeled, for example, with BrdU or [³H]-thymidine, or by introduction of an expression cassette that can express green fluorescent protein, or beta-galactosidase. Alternatively, the modified cells can be detected by their expression of a cell marker that is not expressed by the animal employed for testing (for example, a human-specific antigen when injecting cells into an experimental animal). The presence and phenotype of the administered population of modified cells can be assessed by fluorescence microscopy (e.g., for green fluorescent protein, or beta-galactosidase), by immunohistochemistry (e.g., using an antibody against a human antigen), by ELISA (using an antibody against a human antigen), or by RT-PCR analysis using primers and hybridization conditions that cause amplification to be specific for RNA indicative of a cardiac phenotype.

Modified cells can be included in the compositions in varying amounts depending upon the extent of disease or the condition of the subject. For example, the compositions can be prepared in liquid form for local or systemic administration containing about 10³ to about 10¹² modified cells, or about 10⁴ to about 10¹⁰ modified cells, or about 10⁵ to about 10⁸ modified cells.

One or more RNPs containing a guide RNA or expression vectors that can express one or more guide RNAs, nuclease, or a combination thereof can also be administered with or without the cells.

The guide RNA, nuclease, and/or RNP with or without additional cells may be administered in a composition as a single dose, in multiple doses, in a continuous or intermittent manner, depending, for example, upon the recipient's physiological condition, whether the purpose of the administration is in response to a stressful event or for more sustained therapeutic purposes, and other factors known to skilled practitioners. The administration of the compositions of the invention may be as a single dose, or essentially continuous over a preselected period of time, or it may be in a series of spaced doses. Both local and systemic administration is contemplated.

It will be appreciated that the amounts of guide RNAs, nucleases, RNPs, and/or cells for use in treatment will vary not only with the particular carrier selected but also with the route of administration, the nature of the condition being treated and the age and condition of the patient. Ultimately, the attendant health care provider may determine proper dosage.

The following examples illustrate some of the work involved in the development of the invention. The following publication by the inventors provides further details. Alexanian et al. (A Transcriptional Switch Governing Fibroblast Plasticity Underlies Reversibility of Chronic Heart Disease, bioRxiv (July 2020) doi.org/10.1101/2020.07.21.214874, which is incorporated herein by reference in its entirety).

### Example 1: Materials and Methods

This Example describes some of the materials and methods used in the development of the invention.

### Animal Models

All protocols concerning animal use were approved by the Institutional Animal Care and Use Committees at the University of California San Francisco and conducted in strict accordance with the National Institutes of Health Guide for the Care and Use of Laboratory Animals. Studies were conducted with age-matched male C57Bl/6J. Mice were housed in a temperature- and humidity-controlled pathogen-free facility with 12-hour light/dark cycle.

### Preparation of JQ1

JQ1 was synthesized and purified in the laboratory of Jun Qi (Dana-Farber Cancer Institute), as described by Filippakopoulos et al. (Nature 468, 1067-1073 (2010)). For in vivo experiments, a stock solution [50 mg/ml JQ1 in dimethyl sulfoxide (DMSO)] was diluted to a working concentration of 5 mg/ml in an aqueous carrier (10% hydroxypropyl b-cyclodextrin; Sigma C0926) using vigorous vortexing. Mice were injected at a dose of 50 mg/kg given intraperitoneally once daily. Vehicle control was an equal amount of DMSO dissolved in 10% hydroxypropyl b-cyclodextrin carrier solution. All solutions were prepared and administered using sterile technique. For in vitro experiments, JQ1 was dissolved in DMSO and administered to cells at 500 nM final concentration using an equal volume of DMSO as control.

### Mouse Model of Transverse Aortic Constriction, Echocardiography and Fibrosis Quantification

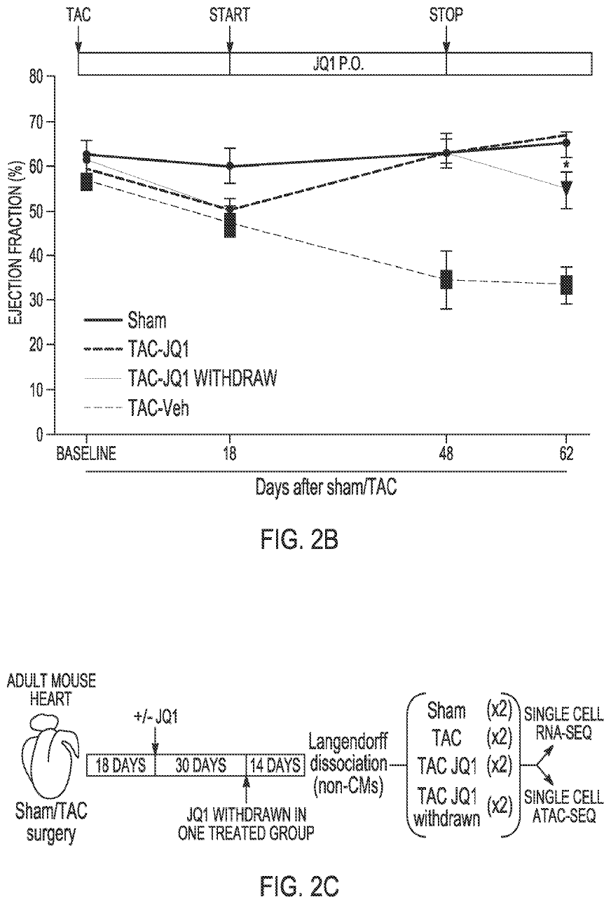

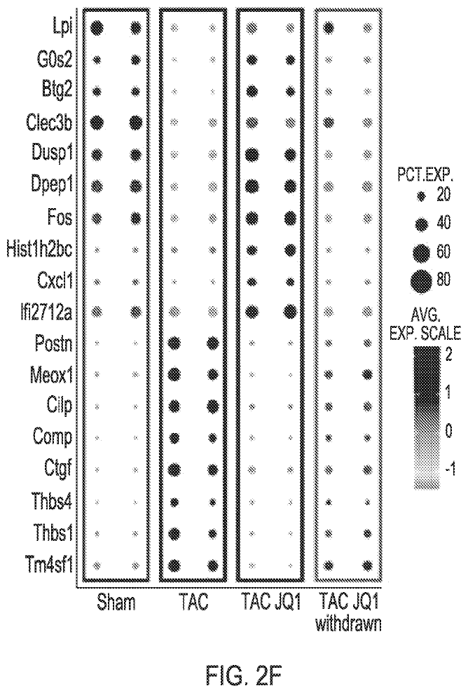

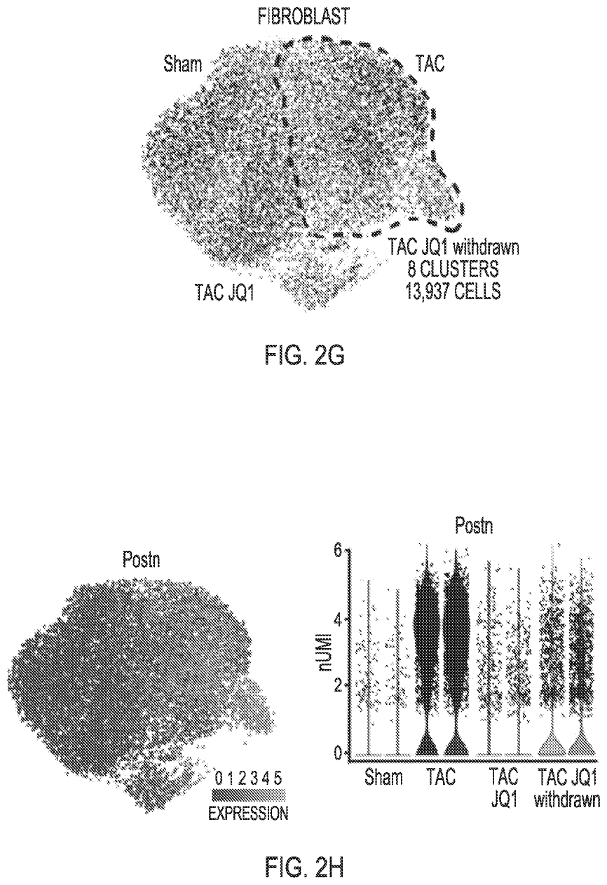

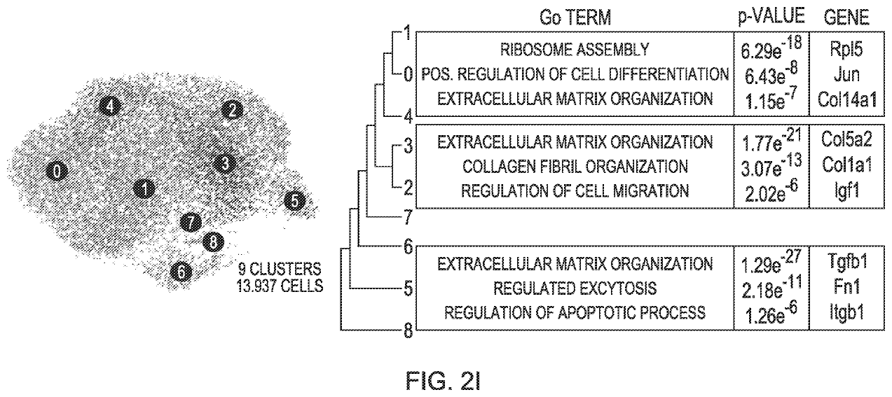

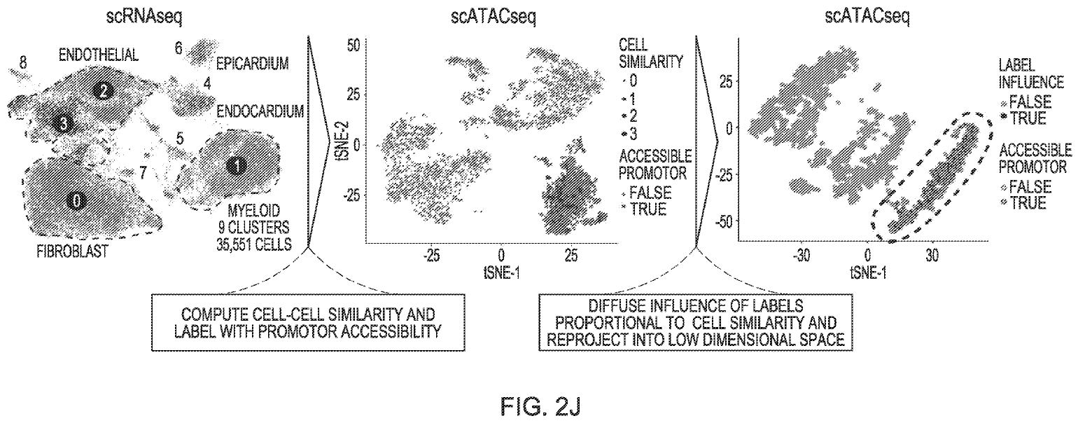

All mice were male C57Bl/6J mice aged 8-10 weeks from The Jackson Laboratory (Stock No: 000664). Mice were placed on a temperature-controlled small-animal surgical table to help maintain body temperature (37° C.) during surgery. Mice were anesthetized with isoflurane, mechanically ventilated (Harvard Apparatus), and subjected to thoracotomy. For transverse aortic constriction (TAC) surgery, the aortic arch was constricted between the left common carotid and the brachiocephalic arteries using a 7-0 silk suture and a 25-gauge needle, as described by Anand (Cell 154: 569-582 (2013)). Intraperitoneal injections of JQ1 (50 mg/kg per day) or vehicle were administered 18 days after TAC surgery and continued as described in FIG. 2B. For sham surgeries, thoracotomy was performed as above, and the aorta was surgically exposed without any further intervention. For echocardiography, mice were anesthetized with 1% inhalational isoflurane and imaged using the Vevo 3100 High Resolution Imaging System (FujiFilm VisualSonics Inc.) and the MX550S probe. Measurements were obtained from M-mode sampling and integrated electrocardiogram-gated kilohertz visualization (EKV) images taken in the ventricle (LV) short axis at the midpapillary level, as described by Anand (2013). Left ventricle areas and ejection fraction were obtained from high-resolution two-dimensional measurements at the end-diastole and end-systole, as described by Anand (2013). For quantifying fibrosis in the TAC JQ1 and TAC JQ1 withdrawn animals, mice were euthanized 62 days post TAC. Hearts were explanted after perfusion with 10 ml of PBS via introduction of a 22.5 G needle into the left ventricle apex and clipping of the right atrium. Hearts were washed with PBS and fixed overnight in 2% PFA followed by dehydration to 100% EtOH in a graded series. For paraffin embedding, hearts were processed in an automated system through successive PBS washes, increasing series of alcohols (Aga), Clear Rite 3@ (Richard-Allan Scientific) and Shandon Histoplast (Thermo Scientific) at 56° C. Hearts were included in paraffin and sectioned transversally (3 μm sections). Sections representative of different z-positions of the ventricles from base of the atria to the apex were dewaxed, rehydrated and Picro Sirius Red stained (ab150681) according the manufacturer's protocol. Sections were diaphanized in xylene and mounted in DPX Mountant for histology (06522, Sigma-Aldrich®). Images were acquired in a Leica DMi8 Widefield with 4× objective and analyzed using FIJI software. In Fiji, images were split into red, green, and blue channels and fibrosis and total tissue section area were measured using the threshold tool into the green and blue channel, respectively. Percentage of fibrosis was normalized to total tissue section area and a total of 24 sections were analyzed per animal. The statistical analysis of the data was performed using Prism 8 with statistical significance determined at p<0.05. Tukey's multiple comparison test was applied. Normality was not verified through D'Agostino-Pearson omnibus normality test and consequently the Independent Samples Mann-Whitney U test was used. A blinded approach (labeling samples with an alphanumeric code) was implemented for analyzing fibrosis.

### Langendorff Perfusion and Cells and Nuclei Isolation for Subsequent Single Cell RNA and ATAC Seq

Cell isolation from mouse hearts was performed as described by Li et al. (J. Vis. Exp. (2014)) with modifications. Briefly, after proper anesthesia level was reached, thoracotomy was performed, and mouse heart was isolated. The isolated heart was cannulated and perfused with perfusion buffer (120.4 mM NaCl, 14.7 mM KCl, 0.6 mM KH₂PO₄, 0.6 mM Na2HPO₄, 1.2 mM MgSO4, 10 mM Na-HEPES, 4.6 mM NaHCO₃, 30 mM taurine, 10 mM 2,3-butanedione monoxime, and 5.5 mM glucose, pH 7.0) in a Langendorff perfusion system (Radnoti 120108EZ) for 5-10 minutes at 37° C. The cannulated heart was then digested by digestion buffer (perfusion buffer with 300 units/mL collagenase II (Worthington Biochemical) and 50 μM CaCl2)) for about 10 min at 37° C. At the end of digestion, the atria and great vessels were removed, and the ventricular tissue was transferred to and gently teased into small pieces in stop buffer (perfusion buffer with 10% fetal bovine serum) at 37° C. After gently pipetting, cell suspension was passed through a 250 uM strainer in a falcon tube and then at 30×g for 3 minutes at room temperature (RT). Then, the supernatant—containing most of the non-cardiomyocytes (CMs)—was divided from the pellet (containing the CM fraction). The non-CM fraction was centrifuged again at 30×g for 3 minutes at RT and the supernatant kept. The supernatant was then filtered with a cell strainer (70 um) and finally centrifuged at 400×g for 3 minutes at RT for eliminating debris. The non-CM pellet was finally resuspended in 1 mL cold PBS 0.5% BSA. 30 k cells were counted with trypan blue using a hemocytometer and then used for subsequent 10× Genomics Chromium single cell RNAseq preparation. For single cell ATAC, 500 k isolated and purified non-CMs were resuspended in OOuL lysis buffer (Tris-HCl 10 mM pH 7.4, NaCl 10 mM, MgCl₂ 3 mM, Tween-20 0.1%, P40 0.1%, Digitonin 0.01%, BSA 1% in Nuclease-free water), pipetted 10 times and kept on ice for 5 minutes. Nuclei were then washed 1 mL 1× PBS with 1% BSA and then centrifuged at for 5 minutes at 4° C. The nuclei pellet was resuspended in 1 mL 1× PBS with 1% BSA and filtered with a 10 uM strainer (pluriSelect #43-50010-00). Nuclei were then counted with DAPI using a hemocytometer and finally 30 k non-CM nuclei used for subsequent 10× Genomics Chromium single cell ATACseq preparation. The CM-fraction was, after the first centrifugation, centrifugated again at 30×g for 3 minutes at RT in stopping buffer and the supernatant was discarded. The CM pellet was finally centrifuged at 400×g for 3 minutes in stopping buffer at RT and after discarding the supernatant, the cell pellet was lysed in Qiazol (miRNeasy kit—Qiagen) for subsequent RNA extraction.

### Bulk RNA Sequencing on Purified Cardiomyocytes

Total RNA from CMs was extracted using miRNeasy kit (Qiagen) according to the manufacturer's instructions and quantified with Nanodrop (Thermo scientific). After RNA quality control with bioanalyzer Agilent 2100 (Agilent Technologies), Paired-end Poly(A)-enriched RNA libraries were prepared with the ovation RNA-seq Universal kit (NuGEN; strand specific) from the Gladstone Genomic core for 9 samples: Sham (×3), TAC-Veh (×3) and TAC-JQ1 (×3). High-throughput sequencing was done using a PE75 run on a NextSeq 500 instrument (Illumina). Reads were mapped to the mm10 reference mouse genome using STAR (v 2.7.3a) and assigned to Ensembl genes. The inventors quantified gene expression using raw counts and kept the protein coding genes that showed an average FPKM value across the samples>0.5 FPKM, where FPKM is the Fragments Per Kilobase of transcript per Million mapped reads. The inventors then performed differential expression gene testing with DESeq2 (v.1.24.0 R package, see Love et al. (Genome Biol. 15, 550 (2014)) using default settings. Statistical significance was set at 5% false discovery rate (FDR; Benjamini-Hochberg).

### Single-Cell Transcriptome Library Preparation, Sequencing, and Processing

Single-cell droplet libraries from the non-CM cell suspension (2× Sham, 2× TAC-Veh, 2× TAC-JQ1 and 2× TAC-JQ1 withdrawn) (FIG. 2C) were generated in the 10× Genomics Chromium controller according to the manufacturer's instructions in the Chromium Single Cell 3′ Reagent Kit v.2 User Guide. Additional components used for library preparation include the Chromium Single Cell 3′ Library and Gel Bead Kit v.2 (PN-120237) and the Chromium Single Cell 3′ Chip kit v.2 (PN-120236). Libraries were prepared according to the manufacturer's instructions using the Chromium Single Cell 3′ Library and Gel Bead Kit v.2(PN-120237) and Chromium 7 Multiplex Kit (PN-120262). Final libraries were sequenced on the NextSeq 500 (Illumina) for a quality control run and then on the NovaSeq (Illumina) for deeper sequencing. All the 8 samples were pooled and sequenced in one single lane. Sequencing parameters were selected according to the Chromium Single Cell v.2 specifications. All libraries were sequenced to a mean read depth of at least 50,000 total aligned reads per cell. Raw sequencing reads were processed using the Cell Ranger v.2.2.0 pipeline from 10× Genomics. In brief, reads were demultiplexed, aligned to the mouse mm10 genome and the absolute number of observed transcripts (UMI counts) were quantified per gene per cell to generate a gene-barcode matrix. Data from the 8 samples were aggregated and normalized to the same sequencing depth, resulting in a combined gene-barcode matrix of all samples.

### Cell Filtering and Cell-Type Clustering for Transcriptomic Analysis

The transcriptomes were sequenced from 35,551 cells that were captured from our 8 samples (FIG. 2C). Filtering and clustering analyses of these cells were performed with the Seurat v.2.2 R package. Cells were normalized for genes expressed per cell and per total expression, then multiplied by a scale factor of 10,000 and log transformed. Low quality cells were excluded from our analyses—this was achieved by filtering out cells with greater than 4,000 and fewer than 1,000 genes and cells with high percentage of mitochondrial genes (higher than 0.2%). Following the filtering step, we normalized the data (NormalizeData function, 10,000 default scale factor) and performed a linear regression on all genes (ScaleData function). The inventors then performed a linear dimensional reduction (RunPCA function). Significant principal components were used for downstream graph-based, semi-unsupervised clustering into distinct populations (FindClusters function) and uniform manifold approximation and projection (UMAP) (Becht et al. Nat. Biotechnol. 37: 38-44 (2018)) dimensionality reduction was used to project the cell population in two dimensions. For clustering, the resolution parameter was approximated based on the number of cells according to Seurat guidelines; a vector of resolution parameters was passed to the FindClusters function and the optimal resolution that established discernible clusters with distinct marker gene expression was selected. The inventors obtained a total of 9 clusters representing the major adult cardiac non-CM cell populations. To identify marker genes driving each cluster, the clusters were compared pairwise for differential gene expression (FindAllMarkers function) using the Likelihood ratio test assuming an underlying negative binomial distribution (negbinom). The inventors then isolated specific clusters (WhichCells function) for subsequent analysis on the fibroblast (cluster 0), myeloid (cluster 1) and endothelial (clusters 2 and 3) populations. Differential expression analysis between samples in the 3 major cell population was performed using the function diffExp=FindMarkers. For visualization of gene expression data between different samples a number of Seurat functions were used: FeaturePlot, VlnPlot and DotPlot. For calculating the normalized expression score, a specific set of genes was passed into the Seurat object to generate a score. For analyzing the gene signature score in every fibroblast cell, the inventors summarized the mean-scaled and z-score normalized gene expression for a given gene in each cell (Hu et al. Nat. Methods 17. 833-843 (2020)). The resulting score was then plotted in Violin plot.

### Single-Cell ATAC Library Preparation, Sequencing, and Processing

After successful nuclei isolation, nuclei were then processed according to the 10× Genomics Single Cell ATAC kit v1.0 (PN-1000110) by first incubating with Tn5 Transposase for 1 hour, followed by GEM generation and barcode amplification using the 10× Genomics Chromium controller according to the manufacturer's instructions. Additional components used for library preparation include the Chromium Chip E Single Cell ATAC kit (PN-1000082) and Chromium i7 Multiplex Kit N, Set A primers (PN-1000084). Final libraries were sequenced on the NextSeq 500 (Illumina) for a quality control run and then on the NovaSeq (Illumina) for deeper sequencing. All the 8 samples (2× Sham, 2× TAC-Veh, 2× TAC-JQ1 and 2× TAC-JQ1 withdrawn) (FIG. 2C) were pooled and sequenced in one NovaSeq single lane. Sequencing parameters were selected according to the Chromium Single Cell ATAC v1.0 specifications. All libraries were sequenced to a median read depth of at least 2,500 fragments/nuclei. Raw sequencing reads were processed using the Cell Ranger ATAC v1.0 pipeline from 10× Genomics. In brief, reads were demultiplexed and aligned to the mouse mm10 genome. As a test of sample quality, a minimum of 70% of fragments overlapped targeted regions as defined by CellRanger. Peaks are then are called on aggregated fragments and then barcodes with fewer fragments than an automatically determined threshold (usually around 200) within these peaks are discarded. The remaining fragments are counted to generate a peak-by-barcode matrix.

Identifying Cell Types in Single Cell ATAC Samples Based on scRNA-Seq

The inventors identified a list of marker genes for each cluster in the scRNAseq data. Then, for each cell in each scATACseq sample, the inventors computed the fraction of that cell's accessible peaks that were in the promoters of each cluster's marker genes. The inventors computed a full cell-cell similarity matrix for each sample using Jaccard similarity of the binarized peak-by-cell matrix generated by CellRanger. FIG. 2J shows an example of mapping cluster 1 from the scRNA-seq data to scATAC-seq sample. For example, the cells marked with darker dots in the right-most panel of FIG. 2J have accessible promoters for Myeloid marker genes. The shading of each cell in FIG. 2J reflects how similar they are to a selected single cell. The inventors then used the method described in Przytycki & Polland to compute global influence scores of each label (BioRxiv(2019). doi:10.1101/847657). For each sample the inventors chose a parameters that maximized the median influence of labels corresponding to three cell types of interest (Fibroblast, Myeloid, and Endothelial) on cells that had accessible promoters for three selected marker genes (Dcn, Lyz2, Fabp4 respectively). The inventors assigned each cell a type based on the label with the highest influence on that cell. FIG. 2J shows that if cells are re-projected into tSNE space using influence scores, cells with the same label cluster together (this projection is only used for illustrative purposes). Using this method, the inventors identified 5,215 fibroblast, 4,278 endothelial, and 3,444 as myeloid cells across eight samples.

### Determining Cell Type Enriched Peaks in Single Cell ATAC

To avoid bias, the inventors computed cell type enriched peaks separately for each sample by comparing accessibility in cells of each type to cells of other types. For each cell type, to determine which peaks are cell type enriched, we repeatedly (ten times each) sampled a set of the same number of cells not of that type and with similar numbers of accessible peaks, and computed a one-tailed Wilcoxon test to determine if each peak was more accessible in the cell type being examined. The inventors then combined sampled p-values using Fisher's method and adjusted for multiple hypothesis correction using the Benjamini-Hochberg procedure. The inventors considered a peak to be cell type enriched in a sample if it was significant at FDR<0.1. For each cell type, the inventors discarded any peaks that did not replicate in at least one other sample. This resulted in 22,467 fibroblast enriched peaks, 12,602 endothelial enriched peaks, and 11,156 myeloid enriched peaks. Note that peaks can be enriched in more than once cell type.

### Computing Single Cell ATAC Normalized Accessibility Scores

For each peak the inventors computed a normalized accessibility score for each cell as one over the total number of accessible peaks in that cell. To compute overall accessibility trends in a cell type and condition, the inventors calculated the fraction of all cell type enriched peaks in a cell that are accessible in that condition. For genome-wide normalized accessibility (e.g. for browser tracks) we instead normalized by number of fragments. To do this the inventors first found all fragments that correspond to barcodes of cells that we want to calculate genome-wide accessibility for. The inventors merge bed files for those cells using the unionBedGraphs function in bedtools2 to create a bedgraph. The inventors assigned an accessibility score to each region of the union bedgraph as the sum of number of fragments in each cell in that region over the total number of fragments for that cell over the number of cells used in the union. To compute the co-accessibility between a peak and a promoter the inventors counted the number of cells in which the promoter and peak were both accessible, normalized by the accessibility of the promoter.

Calculating TF Enrichment Scores from Single Cell ATAC Data

To assess the significance of changes in transcription factor binding between conditions for each cell type the inventors trained a supervised learning model to link transcription factor binding locations to changes in gene expression. The inventors used transcription factors (TF) with known vertebrate motifs included in HOMER, and the top 10 were selected that had the most expressed TFs in TAC in fibroblast, myeloid and endothelial cells. First, all accessible binding sites for each transcription factor were determined in each condition using the “-find” option with the “findMotifsGenome.pl” command in HOMER using the set of distal cell type enriched peaks for that condition. The inventors then generated an g-by-m matrix M^{c} for each condition c, where g is the number of genes and m is the number of transcription factors, by computing the distance from each binding site to the transcription start site of each gene. For each gene i and each binding location k for motif j, the corresponding entry in the matrix was defined as:

$${M}_{i , j}^{c} = {\sum }_{k} \frac{1}{1 + dist ( k , {tss}_{g} )}$$

The difference in motif binding strength between two conditions c₁ and c₂ is then computed as M^{c}^{₁}-M^{c}^{₂}. Then, given a vector Y of length g of log-fold change in expression of genes between the two conditions, the inventors computed the importance of each transcription factor as the difference in change in expression for genes linked to that transcription factor minus genes not linked to that transcription factor while accounting for the effects of other transcription factors by using the targeted Maximum Likelihood Estimation (tMLE) approach described in Stone et al. (Cell Stem Cell 25: 87-102.e9 (2019).

Generating Atlas of Super-Enhancers and Correlate Chromatin Accessibility with Ejection Fraction

To find super-enhancers, the inventors first stitched together all cell type enriched peaks within 12.5 kb of each other that were not separated by a gene using the single cell ATAC data from the TAC Veh samples. The inventors then computed the normalized accessibility for each potential super-enhancer and used the ROSE algorithm (Whyte et al. Cell 153: 307-319 (2013)) to determine the threshold at which regions could be called super-enhancers. The inventors used this method to build a catalog of super-enhancers for fibroblast, myeloid and endothelial cells in the diseased heart (TAC Veh). The inventors then calculated how well each super-enhancer's accessibility correlates with left ventricle ejection fraction (EF). For each super-enhancer we fit a linear model with a vector of EF observations across the four conditions as the response variable and the mean accessibility across the given cell type and across other cells as types as two term vectors. The fitted coefficient of the model for the given cell type is amount of change in EF explained by changes in accessibility in that cell type when controlling for changes in accessibility in other cell types. The inventors call this value the correlation coefficient for each super-enhancer for each cell type, with the significance of each coefficient determined by the p-value of that term in the linear model. For comparison, super-enhancers were generated using the same methodology using H3K27Ac peaks from in vitro unstimulated and TGF-β cell lines (see below).

### Generation of Cardiac Fibroblast Immortalized Cell Line, Culture Condition and TGFβ Stimulation

A Tcf21MCM mouse (Acharya et al., Genesis 49, 870-877 (2011)) was crossed with a Rosa26-Ai6 mouse (Jackson Laboratory stock #007906) to generate a Tcf21^{MCM/+}; Rosa26^{Ai6/+} mouse. At 10 weeks of age, intraperitoneal injection of Tamoxifen (75 mg tamoxifen/kg) was done for 5 days (once a day). Tamoxifen was prepared following Jackson Laboratory guidelines (see website atjax.org/research-and-faculty/resources/cre-repository/tamoxifen#). After 5 days of injection, the mouse was sacrificed the non-cardiomyocyte cells were isolated through Langendorff perfusion (see method section “Langendorff perfusion and cells and nuclei isolation) for subsequent single cell RNA and ATAC seq” for more details. 100 k ZsGreen positive cells were sorted with BD AriaII sorter and cultured for 3 days at 37° C. in a humidified incubator with 5% CO₂ and maintained in fibroblast medium: high glucose DMEM (Life Technologies) supplemented with 10% fetal bovine serum (FBS) (Hyclone, GE Healthcare), 1× Non-Essential Amino Acid (NEAA), 10 U/ml penicillin/streptomycin and 1 mM sodium pyruvate (all from Life Technologies). As primary cells only undergo a pre-determined and finite number of cell divisions in culture, and then enter a state of replicative senescence, the inventors employed a widely used method for immortalizing mammalian cells in culture based on Simian virus 40 (SV40) T antigen expression. For fibroblast immortalization, 3 days after sorting, fibroblast were trypsinized and re-seeded at 5×10⁵ per 100 mm plate in the afternoon. Next morning, fresh media was added to the plates and 2 hours after, and the fibroblasts were infected with 5 μl of SV40 T antigen expressing VSV-G pseudotyped lentiviral particles (Alstem, #CILVO1) per 100 mm plate in the presence of Polybrene (added to a final concentration of 5 μg/ml) following manufacturer's instructions. The next day, the medium containing the viral supernatant was removed and fibroblasts were switched back to high glucose DMEM (Life Technologies) supplemented with 10% fetal bovine serum (FBS) (Hyclone, GE Healthcare), 1× Non-Essential Amino Acid (NEAA), 10 U/ml penicillin/streptomycin and 1 mM sodium pyruvate (all from Life Technologies). After 72 hours, the cells were trypsinized and split 1:2 in two 100 mm plates. Then, Puromycin was added to the medium (final concentration of 1 ug/ml) to positively select for infected cells for stable cell-line generation. After 10 days of puromycin selection, multiple clones were picked for expansion. For daily cell-line maintenance, fibroblasts were split every 2-3 days and the media was changed every other day. Same media was used for HEK-293T cell culture for lentiviral production (see next sections). For the TGF-β stimulation, fibroblasts were seeded at 1×10⁵/well of 6 well plate at day 1. On day 2, the media was changed to the same basal media with 0.5% FBS. On the day 3, TGF-β1 (Peprotech #100-21C) was added into the media at a concentration of 10 ng/mi. Cells were collected on day 5 for downstream analysis.

### RNA Extraction, RT-PCR, and Real-Time PCR Analysis

Quantitative RT-PCR. Cells were harvested in TRIzoI™ LS reagent (Invitrogen) and total RNA was extracted using the Direct-Zol RNA kit (Zymo Research) according to manufacturer instruction. 500 ng of RNA was converted to cDNA using SuperScrip™ III First-strand Synthesis SuperMix for qRT-PCR (Invitrogen). For Taqman real-time PCR, 1/50 cDNA was applied for quantitative PCR reaction using Taqman Universal PCR master mix (Life technologies). The PCR was conducted in 7900HT Fast Real-Time system (Applied Biosystem). The Taqman probes are listed in the ‘Taqman probes table’. For eRNA expression analysis, 1/30 cDNA was applied for quantitative PCR reaction using SsoAdvanced Universal SYBR green supermix (Bio-Rad). Primer sequences are listed in the ‘Syber primers for enhancer RNAs (eRNAs) table’. All gene expressions were normalized with Actb gene.

### Precision Nuclear Run-on Sequencing (PROseq)

PRO-seq experiments were performed as reported by Kwak et al. (Science 339: 950-953 (2013)) with a few modifications. Briefly, 3 million Cardiac fibroblasts were cultured as described previously in this method. After 48 h of TGFβ treatment, cells were washed 3 times with cold PBS and then sequentially swelled in swelling buffer (10 mM Tris-HCl pH7.5, 2 mM MgCl₂, 3 mM CaCl₂) for 10 min on ice, harvested, and lysed in lysis buffer (swelling buffer plus 0.5% NP-40, 20 units of SUPERase-In, and 10% glycerol). The resultant nuclei were washed two more times with 5 ml lysis buffer, resuspended in 200 μl of freezing buffer (50 mM Tris-HCl pH8.3, 40% glycerol, 5 mM MgCl₂, 0.1 mM EDTA), and split in two equal aliquots, (aliquot A: no decapping; aliquot B: decapping). For the run-on assay, resuspended nuclei were mixed with an equal volume of reaction buffer (10 mM Tris-HCl pH 8.0, 5 mM MgCl₂, 1 mM DTT, 300 mM KCl, 20 units of SUPERase-In, 1% sarkosyl, 100 M A/GTP, 100 μM biotin-11-C/UTP (Perkin-Elmer) and incubated for 5 min at 30° C. The resultant nuclear-runon RNA (NRO-RNA) was then extracted with TRIzol® LS reagent (Life Technologies, Cat #10296-028) following manufacturer's instructions. NRO-RNA was fragmented to 200-500 nt by alkaline base hydrolysis on ice for 30 min and neutralized by adding 1×volume of 1 M Tris-HCl pH 6.8, Excessive salt and residual NTPs were removed by using P-30 column (Bio-Rad, Cat #732-6250). Fragmented nascent RNA was precipitated twice using 10 μl of MyOne Streptavidin C1 dynabeads (Invitrogen, Cat #65001) following the manufacturer's instructions to enrich for the biotinylated RNA. The beads were washed twice in high salt (2 M NaCl, 50 mM Tris-HCl pH 7.5, 0.5% Triton X-100, 0.5 mM EDTA), once in medium salt (1M NaCl, 5 mM Tris-HCl pH 7.5, 0.1% Triton X-100, 0.5 mM EDTA), and once in low salt (5 mM Tris-HCl pH 7.5, 0.1% Triton X-100). Bound RNA was extracted from the bead using Trizol (Invitrogen, Cat #15596-018) in two consecutive extractions, and the RNA fractions were pooled, followed by ethanol precipitation.

At this step, only aliquot B has been incubated 1 hour at 37° C. with RNA 5′ Pyrophosphohydrolase (Rpph, NEB M0356S) in decapping mix (1× Thermopol Buffer NEB(B9004S), 20 units of SUPERase-In), to remove 5′ cap from nascent RNA. Decapping reaction was stopped by heating the samples 5 min. at 65° C. and RNA has been extracted using Trizol (Invitrogen, Cat #15596-018) followed by ethanol precipitation. Then, 5′ phosphorylation was performed on both aliquots by incubation in T4 reaction mix (T4 polynucleotide kinase (NEB #M0201L), 1× PNK Buffer (NEB #B201S), 10 mM ATP (NEB #B0706A)) for 1 h at 37° C. RNA has been extracted using Trizol (Invitrogen, Cat #15596-018) followed by ethanol precipitation. Libraries were generated using the NEBNext® Multiplex Small RNA Library Prep Set. (NEB, Cat #E7300S) following the manufacturer's instructions. The cDNA products were separated on a 10% polyacrylamide TBE-urea gel and only those fragments migrating between 200-500 bp were excised and recovered by gel extraction. Finally, libraries were quantified by Qubit and sent to sequence SR75 bp on a HiSeq 4000 platform (Illumina).

### PROseq Analysis

FastQ files resulting from the deep sequencing were cleaned from low quality reads using Trimmomatic (Bolger et al. Bioinformatics 30: 2114-2120 (2014)). Trimmed FastQ file from aliquot A (no decapping) and aliquot B (decapping) have been aligned together to the reference genome (mm10) using Bowtie2 (see website bowtie-bio.sourceforge.net/bowtie2/). The resulting alignment file has been used to create a Tag Directory for downstream analysis using homer (see website homer.ucsd.edu/homer/). Differential expression of coding genes and distal elements has been performed using homer commands analyzeRepeats.pl and getDiffExpression.pl (see homer.ucsd.edu/homer/ngs/diffExpression.html). A threshold of minimal transcription was used to select differentially transcribed genes (x>20 average row counts in all samples for distal elements, x>40 average row counts in all samples for protein coding genes). Histogram plots have been generated using the histogram mode of the command annotatePeaks (see homer.ucsd.edu/homer/ngs/annotation.html). The inventors used the tool called makeMetaGeneProfile.pl to generate Metagene histograms (see homer.ucsd.edu/homer/ngs/quantification.html). Finally, enhancers have been called based on PROseq tag density using homer commands findPeaks and getDistalPeaks.pl (see homer.ucsd.edu/homer/ngs/groseq/groseq.html).

### CRISPR Interference (CRISPRi) for Sequence-Specific Repression

For repressing enhancer activity, CRISPRi was used. The lentiviral plasmid, pHR-SFFV-KRAB-dCas9-mcherry (gift from Dr. Jonathan Weissmen, Addgene: 60954) was used. For constructing gRNA lentiviral vector, we modified pU6-sgRNAEF1Alpha-puroT2A-BFP (gift from Dr. Jonathan Weissmen, Addgene: 60955) by replacing the puromycin gene with Hygromycin gene and made pU6-sgRNAEF1Alpha-HygT2A-BFP. Pairs of synthesized gRNA oligos (‘CRISPRi guide RNAs targeting enhancers table’) with 5′ and 3′ overhangs were annealed and sub-cloned into BstXI and BipI double digested pU6-sgRNAEF1Alpha-HygT2A-BFP by T4 ligase mediated ligation. The construct was sequencing verified (Quintara Bio, Berkeley, Calif., USA). The gRNAs for repressing enhancer peaks were chosen by the program Chopchop (see chopchop.cbu.uib.no). For generating the lentiviral particles, 2×10⁶ HEK-293T cells were seeded on a 100 mm plate one day prior the transfection and cultured in 10 ml fibroblast media. On the day of transfection, the old media was replenished with 8 ml of fresh media, then 5 μg of desired lentiviral vector was co-transfected with 2.5 μg of envelope protein vector pMD2.G (Addgene:12258), and 2.5 μg of the packaging vector psPAX2 (Addgene: 12260) into HEK 293T cells using 59 ul of FUGENE HD transfection reagent (Promega, San Luis Obispo, Calif., USA) following the manufacturer's instruction. 48 hours after transfection, supernatant was collected and 12 ml of fresh media was added to the plate and culture for another 24 hours for secondary collection of the supernatant. All supernatants were filtered through 45 μm PVDF syringe filter (Thermo Fisher Scientific) to remove the cell debris contamination. The obtained supernatant was then used for performing transduction on cardiac fibroblasts. Firstly, the inventors generated a fibroblast CRISPRi control line. We transduced the fibroblasts with lentiviral vector that expressed the KRAB-dCas9-mcherry. The inventors then collected the fibroblasts and single cell sorted in to 96 well plate by flow cytometry (BD Arial II) and generated a clonal fibroblast line expressing the CRISPRi machinery. The expression of the dCas9 was confirmed by western blot (data not shown) and the fibroblast line with the highest dCas9 expression (referred as clone C1) was used for subsequent experiments. For generating the fibroblast lines with targeted repression of the enhancer regions in this study, the C1 clone was transduced with three gRNA viral vectors that target the desired region (one gRNA targeting the center of the enhancer peak, the other targeting the two extreme parts of the peak). Pure polyclonal population of targeting gRNAs were selected by hygromycin selection at 200 μg/ml for 7 days. These gRNA-C1 cell lines were then used together with the C1 control line for TGF-β stimulation (see other sections herein).

### Generation of Meox1 Enhancer Peak 9/10 Deletion in Cardiac Fibroblast

To generate the deletion in immortalized cardiac fibroblast, the inventors used CRISPR/Cas9 ribonucleoprotein complex (RNP) mediated genome editing. Two crRNAs oligo (upper-crRNA: AGGCTTCACTTACCCTAGAC (SEQ ID NO:18); Down-crRNA: CAATAATGGGCTCTGTAAGG (SEQ ID NO:19)), flanked to the desired deletion region (Mm Chr17: chr17:43, 591, 446-43,592,491) were synthesized together with a TracRNA oligo and obtained from Integrated DNA Technologies (IDT). Before transfection, 240 pmol from each crRNAs oligos were annealed to same amount of the TracRNA to form guide RNA complexes by heating the mixture at 95° C. for 5 minutes and cooling to room temperature for 30 minutes. Then guide RNA mixture was mixed with 40 pmol spCas9 protein (Macro Lab, UCB) and incubated with 20 ul of transfection solution from P2 primary cell 4D-nuclofector x Kit (Lonza) in room temperature for 15 minutes.

The final Cas9:gRNA RNP complex were nucleofected to 1×10⁵ cardiac fibroblast cells using Prog EN-150 in Lonza 4D nucleofector (Lonza). After transfection, cells were seeded to 1 well of 96 well plate and cultured for two days. On the day 3, cells were dissociated by trypsin and suspended in 1 ml of fibroblast culture medium. The cells were counted, and 300 cells were seeded in to three 96 well plates to get a cell per well. Only the wells with a single cell were marked after seeding. Two weeks later, when clones became confluent, a quarter of the cells was passed for continuing culture, other three of fourth was collected for extracting genomic DNA. Detection of deletion was performed by PCR analysis using pair of primer (Table: Primers for detection of Meox1 enhancer Peak 9/10 deletion) chosen from upper and downstream of the deletion region using PrimSTAR polymerase (TAKARA Bio). The PCR products were sequenced by sanger sequencing to confirm the presence or absence of the deletion. The clones were further analyzed with digital PCR to identify the biallelic Peak9/10 deletion clone. Digital PCR (ddPCR) assay mix contain 50 to 100 ng genomic DNA, 1× ddPCR supermix (BioRad Laboratories), WT primers and Del primers at the final concentration of 900 nM and 220 nM with FAM or HEX labeled probes in a 22 ul final volume was prepared. 20 ul of assay mix and 70 ul of ddPCR droplet oil (BioRAd Laboratories) were transferred onto a QX100/200 DG cartridge (BioRad Laboratories), then loaded into the QX100 Droplet Generator (BioRad Laboratories). The generator pulling individual samples and oil through a flow-focusing junction to produce water-in oil droplets. 40 ul of the oil and sample droplets emulsions were then transferred into a 96 well plate and went through PCR reaction in T100 Thermo Cycler (BioRAd Laboratories) for 95° C. for 10 minutes, 94° C. for 1 min and 60° C. for 1 min (repeated for 40 cycles), then 98° C. for 10 mins. After PCR completed, the plate was then transferred to a QX200 Droplet Reader (BioRad Laboratories) and analyzed with QX software (BioRad Laboratories). The clones with and without deletion were used for TGF-β1 stimulation study.

### Generation of MEOX1-HA Cardiac Fibroblast Line

The pHR-HAtag-mMeox1 vector was constructed by PCR amplifying the HA tag-mMeox1 fragment from the vector HA-tag-MEOX1 mouse (Twist Biosciences). For generating the pHR-HAtag-mMEOX1 construct, the KRAB and dCas9 cassettes from the pHR-SFFV-KRAB-dCas9-mCherry vector were replaced with the HA-tag-MEOX1 cassette using a Cold-fusion cloning kit from SBI System BioSciences (Palo Alto, Calif., USA) by following the instruction provided by the manufacturer. The construct was verified by sequencing. For generating the lentiviral particles, the same procedure described in the section “CRISPR interference (CRISPRi) for sequence-specific repression” was used. The obtained supernatant was then used for performing transduction on immortalized cardiac fibroblasts with the lentiviral vector that express HA-tag-MEOX1 and mCherry. Pure polyclonal population of HA-tag-MEOX1 fibroblasts were sorted by flow cytometry (BD Arial II) for stable mCherry expression. This HA-tag-MEOX1 fibroblast line was used for subsequent chromatin immunoprecipitation followed by sequencing (ChIPseq).

### ChIP Assay, Library Preparation for Sequencing and Analysis

For ChIP experiments, 10×10⁶ cardiac immortalized fibroblasts (HA-tag-MEOX1 fibroblast for MEOX1 ChIPseq) in unstimulated and TGFβ-treated condition were pelleted and suspended in 10 ml DMEM and cross-linked in 1% formalin solution (Thermo Fisher Scientific) by rocking in room temperature for 10 minutes. Then glycine (final concentration 0.125M) was added to quench the cross-link for 5 minutes. Samples were centrifuged at 1000 ref for 5 minutes at 4° C. Cells were washed with 10 ml of cold 1× PBS supplemented with proteinase inhibitors and phosphatase inhibitors (Roche #4693132001) and the pellets were snap frozen in liquid nitrogen. All samples were stored at −80° C. until use. When ready, cell pellets were incubated in cell lysis buffer (20 mM Tris-HCl, pH 8, 85 mM KCl, 0.5% NP-40, protease inhibitors for 10 min on a rotator at 4° C. Nuclei were isolated by centrifugation (2,500×g, 5 min, 4° C.), resuspended in nuclear lysis buffer (50 mM Tris-HCl, pH 8, 10 mM EDTA, pH 8, 1% SDS, protease inhibitors) and incubated on a rotator for 30 min at 4° C. Chromatin was sheared using a Covaris S2 sonicator (Covaris Inc) for 20 min (60 s cycles, 20% duty cycle, 200 cycles/burst, intensity=5) until DNA was in the 200-700 base-pair range. Chromatin was diluted 3-fold in ChIP dilution buffer (0.01% SDS, 1.1% Triton X-100, 1.2mMEDTA, 16.7mMTris-HCl, pH 8, 167 mM NaCl, protease inhibitors) and incubated with 3 ul of anti-HA antibody (Abcam #9110) or 2 ul of anti-H3K27ac (Abcam #4729) or 2 ul of anti-H3K9m3 (Abcam #8898) at 4° C. overnight under rotation. Antibody-protein complexes were immunoprecipitated using Pierce Protein A/G magnetic beads at 4° C. for 2 h under rotation. Beads were washed five times (2-min/wash under rotation) with cold RIPA buffer (50 mM HEPES-KOH, pH 7.5, 500 mM LiCl, 1 mM EDTA, 1% NP-40, 0.7% Na-deoxycholate), followed by one wash in cold final wash buffer (1×TE, 50 mM NaCl). Immunoprecipitated chromatin was eluted at 65° C. with agitation for 30 min in elution buffer (50mMTris-HCl pH 8.0, 10mMEDTA, 1% SDS). High-salt buffer (250 mM Tris-HCl, pH 7.5, 32.5 mM EDTA, pH 8, 1.25M NaCl) and Proteinase K (New England Biolabs Inc (NEB)) were added and crosslinks were reversed overnight at 65° C. Samples were treated with RNase A, and DNA was purified with AMPure XP beads (Beckman Coulter cat #A63881). For following ChIPseq, fragmented ChIP and input DNA were end-repaired, 5′-phosphorylated and dA-tailed with NEBNext Ultra 11 DNA Library Prep Kit for Illumina (NEB, E7645). Samples were ligated to adaptor oligos for multiplex sequencing (NEB, E7335), PCR amplified, and sequenced on an Illumina NextSeq 500 at the Gladstone Institutes. For ChIP qPCR, ChIPed and input DNA were amplified using primers spanning defined region in the Postn and Meox1 locus (see table for primer sequences) and RT-qPCR was run.

For the ChIPseq analysis, trimming of known adapters and low-quality regions of reads was performed using Fastq-mcf. Sequence quality control was assessed using FastQC (see www.bioinformatics.babraham.ac.uk/projects/fastqc/). Alignment to the mm10 reference genome was performed using Bowtie 2.2.4 (Langmead et al., Nat. Methods 9, 357-359 (2012)). Replicates were tested for correlation using multiBamSummary from deeptools 3.5.0 (Ramirez et al. Nucleic Acids Res. 44, W160-(2016)). Peaks were called using GEM (Guo et al. PLoS Comput. Biol. 8, e1002638 (2012)). Read counts per peak were generated with featureCounts (Liao et al. Bioinformatics 30, 923-930 (2014)) and normalized to account for differences in sequencing depth between samples using upper quartile normalization separately for the ChIP and input sample. For MEOX1 anti-HA and H3K27ac ChIPseq, regions enriched with MEOX1 or H3K27ac were determined using empirical Bayes F-tests for a quasi-likelihood negative binomial generalized log-linear model of the count data as implemented in edgeR. Specifically, the inventors tested for a significant (i.e., non-zero at FDR<5%) log 2 fold-increase in normalized peak signal for ChIP versus the corresponding input sample. Region intersections were found using BEDTools (Quinlan & Hall, Bioinformatics 26, 841-842 (2010)). MEOX1 coverage distributions were calculated first by first computing a read normalized average across all three replicates per condition (unstimulated and TGFβ) using bamCompare from deeptools 3.5.0 (this also outputs a bigwig file that is used for plotting tracks) and then scored using computeMatrix from deeptools 3.5.0 with the scale-regions options. Coverage for H3K27Ac regions was calculated similarly, except a bed file was first generated for H3K27Ac data with ranges centered on each peak extending 1 kb upstream and downstream; computeMatrix was run on these bed files without the scale-regions option. For calculating how many protein coding gene loci overlap with MEOX1 peaks the inventors kept peaks present in at least two of the three replicates in the MEOX1 ChIP in TGFβ treatment.

### Circularized Chromosome Conformation Capture (4C)

4C-seq experiments were largely performed as reported by Stadhouders et al. (Nat. Protocol. 8: 509-524 (2013)) with a few modifications. Briefly, 10 million cardiac fibroblasts were cultured as previously described in this method. After 48 h of TGF-β treatment, cells were washed 3 times with cold PBS and then sequentially cross-linked with 1% formaldehyde for 10 min. The reaction was quenched by addition of glycine to a final concentration of 0.125 M. Cells were washed once again with cold PBS and nuclei were extracted by incubation 10 min in ice with 5 ml of ice-cold lysis buffer (10 mM Tris-HCl, pH 8.0, 10 mM NaCl, 0.2% NP-40+protease inhibitor). Nuclei were resuspended in restriction enzyme buffer (NEB Cat #B7006S) and incubated with 0.3% SDS for 1 h at 37° C. and further incubated with 2% Triton X-100 for 1 h. 400U of DpnII restriction enzyme (NEB Cat #R0543S) was added and incubated overnight. Restriction enzyme was heat inactivated at 65° C. for 20 min. Ligation of DNA regions in close physical proximity was performed using 1000U of T4 DNA ligase (NEB M0202M) overnight. The following day, 300 μg of proteinase K were added and decrosslinking was performed at 65° C. overnight. After de-crosslinking, DNA was purified using phenol/chloroform precipitation and the second digestion was performed by adding 400U of NiaIII restriction enzyme (NEB Cat #R0125S), then incubation at 65° C. for 4 h. After the second digestion, ligation of DNA was performed again using T4 DNA ligase as described above. 4C-seq libraries were amplified using PCR with primer containing partial Illumina sequence adaptors (1^{st} primer: gttcagagttctacagtccgacgatc (SEQ ID NO:20); 2^{nd} primer: agacgtgtgctcttccgatct (SEQ ID NO:21). The first primer was designed on each viewpoint and the second primer designed beside the closest NiallI cutting site to the viewpoint. The primer sequences used are listed in the ‘4C primers table’.

Full-length Illumina sequencing adaptors and barcodes were added by the second round of PCR. Finally, libraries were quantified by Qubit and library quality checked with bioanalyzer Agilent 2100 (Agilent Technologies). High-throughput sequencing was done using a SE75 run on a NextSeq 500 instrument (Illumina). Sequencing results have been analyzed as described by Krijger et al. (Methods 170, 17-32 (2020)). Peak calling over background has been performed using de function “doPeakC” directly on the rds files produced by the pipeline as described by Krijger (2020).

siRNA Transfection on Cardiac Fibroblasts

1×10⁵ of immortalized fibroblasts or primary adult cardiac fibroblasts were seeded before the day of transfection in 6 well plate. On the day of transfection, the wells for the subsequent TGFβ treatment were switched on fibroblast medium with 0.5% FBS, while the Unstimulated wells were switched with normal fibroblast medium with 10% FBS. Cells were transfected with 15 nM of mouse Meox1, siBrd2, siBrd3, siBrd4, siSmad2 or siSmuad3 siRNA or 15 nM siRNA Control (all siRNA from Sigma, reference numbers are provided below in tables), using 7 μl of Lipofectamine™ RNAiMAX transfection reagent (Thermo Fisher) for each well according to the manufacturer's instruction. 24 hours after transfection, the wells with 0.5% FBS medium were treated with TGF-β1 (detailed procedure described in another section).

### Smooth Muscle Actin Protein Immunostaining

Unstimulated and TGBβ-treated fibroblasts (with siRNA control or Meox1) were stimulated as described above (see “Generation of cardiac fibroblast immortalized cell line, culture condition and TGFβ stimulation” section for more details”). For alpha smooth muscle actin staining, cells were fixed with 4% PFA for 15 min, washed and permeabilized for 5 min in 0.2% Triton X-100 (Sigma). After blocking with MOM blocking solution (VECTOR Laboratories), cells were then incubated for 30 min with a primary antibody against alpha smooth muscle actin (aSMA; DAKO, M0851; 1:100) followed with a donkey anti-mouse IgG 555 antibody (Invitrogen; 1:400) and Hoechst (Thermo Scientific, 62246; 1:10000) stain diluted in MOM diluent solution (VECTOR Laboratories). Imaging was performed on a Zeiss Axio Observer Z1 using the same acquisition settings across samples and the fold change of aSMA expression quantified using FIJI. aSMA average fluorescence intensity was quantified using the Measure tool (Set Measurements ‘mean grey value’) and normalized to the total cell number. Total cell number were quantified by analyzing the total nuclei per field using threshold, watershed and analyze particle tools. A total of 10 regions of interests were analyzed per well. The statistical analysis of the data was performed using Prism 8 with statistical significance determined at p<0.05. Tukey's multiple comparison test was applied. Normality was not verified through D'Agostino-Pearson omnibus normality test and consequently the Independent Samples Mann-Whitney U test was used. A blinded approach (labeling samples with an alphanumeric code) was implemented for analyzing fibroblast aSMA expression.

### Primary Adult Cardiac Fibroblast Isolation and Culture

Primary adult mouse ventricular fibroblasts (AMVFs) were prepared with minor modifications to the protocol described previously (Travers et al. J. Am. Coll. Cardiol. 70, 958-971 (2017)). Briefly, mice were anesthetized with isoflurane and administered 100 μL of heparin (100 U/mL) via intraperitoneal injection. The hearts were excised and immediately suspended on a Langendorff apparatus by cannulation of the aortic root and perfused at a constant rate of 4 mL/min at 37° C. starting with 4 minutes of perfusion buffer (113 mM NaCl, 4.7 mM KCl, 0.6 mM KH2PO₄, 0.6 mM Na2HPO₄, 1.2 mM MgSO₄, 10 mM HEPES, 12 mM NaHCO₃, 10 mM KHCO₃, 30 mM Taurine, 10 mM 2,3-Butanedione monoxime, 5.5 mM D-(+)-glucose, pH 7.4). Subsequently, enzymatic digestion was achieved by 3 min of perfusion with calcium-free digestion buffer (400 units/mL of collagenase II in perfusion buffer; Worthington LS004177) followed by 12 min of perfusion with digestion buffer containing 50 μM CaCl₂). Hearts were removed from the perfusion apparatus, atria were removed, and ventricles placed in Stopping Buffer (10% FBS and 12.5 μM CaCl2 in perfusion buffer). Ventricles were gently mechanically disrupted using transfer pipettes until tissue was sufficiently digested. The cell suspension was filtered through a 250 μm mesh and CMs were allowed to settle by gravity for 10 min; the supernatant, containing the first non-CM fraction, was collected. CMs were resuspended in an additional 10 mL Stopping Buffer and subsequently allowed to settle for 10 minutes. Supernatant was collected and both non-CM fractions were centrifuged at 500×g for 5 min. CMs were discarded. Non-CMs were resuspended, combined, and plated in growth medium consisting of DMEM/F12 media (Corning 10-092-CV) supplemented with 10% BenchMark™ FBS (Gemini Bio-Products 100-106), 1% Penicillin Streptomycin L-Glutamine (Corning 30-009-Cl) and 1 μmol/L ascorbic acid. Upon reaching 80% confluency, AMVFs were passaged once to P1, in an attempt to deter spontaneous activation, and plated appropriately for downstream assays.

### Collagen Gel Contraction Assay

Compressible collagen matrices were prepared in 24-well plates using PureCol EZ Gel Solution (Advanced BioMatrix 5074) by incubating at 37C for 1.5 hrs. Passage 1 AMVFs suspended in serum-supplemented growth medium were seeded (150,000 cells/gel) on the collagen gels for 24 hrs prior to equilibration by serum deprivation (0.1% FBS) overnight. During serum-starvation, cells were also transfected with an siRNA directed against murine Meox1 (or a negative control siRNA) using Lipofectamine™ RNAiMAX Transfection Reagent (ThermoFisher Scientific 13778030) according to the manufacturer's instructions. At the initiation of contraction, gels were released from the walls of the well, transfection reagent was removed, and cells were treated with 10 ng/mL TGF-β1 (Fisher Scientific 50725143) for 72 hrs. Well images were captured every 24 hrs; gel area for each well was determined using ImageJ software and data are reported as percent contraction.

### EdU (5-Ethynyl-2′-Deoxyuridine) Incorporation Assay

Passage 1 AMVFs were seeded in 12-well plates containing glass coverslips at a density of 20,000 cells/well for 24 hrs, followed by equilibration in serum-starvation media (0.1% FBS) overnight. During serum-starvation, cells were also transfected with an siRNA directed against murine Meox1 (or a negative control siRNA) using Lipofectamine™ RNAiMAX Transfection Reagent (ThermoFisher Scientific 13778030) according to the manufacturer's instructions. Media was exchanged and cells were stimulated with 10 ng/mL TGF-β1 (Fisher Scientific 50725143) for 48 hrs; AMVFs were simultaneously incubated with 10 μM 5-ethynyl-2′-deoxyuridine (EdU) to label proliferative cells over the 48-hour period of stimulation. AMVFs were fixed in 3.7% formaldehyde and EdU-positive cells were detected using the Click-iT® EdU Imaging Kit for Imaging (ThermoFisher Scientific C10337) according to the manufacturer's protocol. Coverslips were mounted using ProLong™ Diamond Antifade Mountant (Invitrogen P36961) and allowed to cure overnight prior to imaging on a Keyence BZ-X710 fluorescence microscope. Percent EdU incorporation was determined by quantifying the percent of EdU positive cells relative to the number of nuclei detected per field.

Human Fibroblasts from Lung, Liver, and Kidney

Human fibroblasts from lung (ATCC, #CRL-4058), liver (CELL APPLICATIONS INC, #712-05f) and kidney (Cell Biologics, #H-6016) were passaged and grown like the mouse cardiac fibroblasts (see “Generation of cardiac fibroblast immortalized cell line, culture condition and TGFβ stimulation” section for more details”). In brief, human fibroblasts were seeded at 1×10⁵/well of 6 well plate at day 1. On day 2, the media was changed to the same basal media with 0.5% FBS. On the day 3, TGF-β1 (Peprotech #100-21C) was added into the media at a concentration of 10 ng/ml. JQ1 was added at a final concentration of 0.5 uM. Cells were collected on day 5 for RT-qPCR and gene expression analysis.

### Gene Ontology (GO) Analysis

GO analysis on distal elements was performed using GREAT (see great.stanford.edu/public/html/). GO analysis on protein coding genes was performed using Enrichr (Chen et al. BMC Bioinformatics 14, 128 (2013)).

### Statistics and Reproducibility

Standard statistical analyses were performed using GraphPad Prism 8. When several conditions were to compare, we performed a one-way ANOVA, followed by Tukey range test to assess the significance among pairs of conditions. When only two conditions were to test, we performed Student's t-test. All the p-values related to the figures showing chromatin accessibility were obtained with two-tailed Wilcoxon tests. For all quantifications related to cardiac function, gene expression by RT-qPCR, collagen contraction and EdU incorporation, the means±SEM are reported in the figures. For gene expression data by RT-qPCR analysis or RNAseq FPKM, the number of replicates is indicated as data points in the graphs. The level of significance in all graphs is represented as follow. * P<0.05, ** P<0.01, *** P<0.001, **** P<0.0001.

TABLE 2
Gene Reference Numbers

| Gene | Reference # |
|---|---|
| Actb | Mm01205647 |
| Meox1 | Mm00440285 |
| Postn | Mm01284919 |
| Col8a1 | Mm01344185 |
| Ctgf | Mm01192933 |
| Wisp2 | Mm00497471 |
| Smad2 | Mm00487530 |
| Smad3 | Mm01170760 |
| Brd2 | Mm01271171 |
| Brd3 | Mm01333576 |
| Brd4 | Mm00480394 |
| ACTB | Hs01060665 |
| MEOX1 | Hs00244943 |

TABLE 3
Syber primers for enhancer RNAs (eRNAs) table

| Gene | Forward sequence | Reverse sequence |
|---|---|---|
| Postn | ATTCCAAGCCTG | AGACACTGGCTT |
| Peak 8 | AAAGAGCA | GGCTTAGG |
| eRNA | (SEQ ID NO: 22) | (SEQ ID NO: 23) |
| Postn | CAGATGAGCCAC | CCAGGCAGATGA |
| Peak | AAGAGGTG | CAGTCAGA |
| 10/11 | (SEQ ID NO: 24) | (SEQ ID NO: 25) |
| eRNA | | |
| Postn | ATGGTTCCCTTC | AAGCGTTGCCCT |
| Peak 19 | AACCACTG | CAGTATGT |
| eRNA | (SEQ ID NO: 26) | (SEQ ID NO: 27) |
| Meox1 | GCTTGGATCAG | TGGGTCAGGTTC |
| Peak 5 | CTCCCTACA | AAGACTCC |
| eRNA | (SEQ ID NO: 28) | (SEQ ID NO: 29) |
| Meox1 | GCTGGGGTACA | GGCCACAAGAC |
| Peak | GGCATACAC | ACTCCAAGT |
| 9/10 | (SEQ ID NO: 30) | (SEQ ID NO: 31) |
| eRNA | | |
| Meox1 | GGGAGAGTAGTG | ACTCATGGGGAG |
| Peak 13 | CGGAACAG | CTGCTGTA |
| eRNA | (SEQ ID NO: 32) | (SEQ ID NO: 33) |

TABLE 4
CRISPRi guide RNAs targeting enhancers table

| | Location | |
|---|---|---|
| Enhancer | in the | |
| Peaks | peak | Guide RNAs |
| Postn Peak 8 | Side | CTGCGCTGCACTTTAGAAGT |
| | | (SEQ ID NO: 34) |
| Postn Peak 8 | Side | TTGGAATTTGAGCCAATGG |
| | | (SEQ ID NO: 35) |
| Postn Peak 11 | Center | TTGGTCATCGGGAGACTCCG |
| | | (SEQ ID NO: 36) |
| Postn Peak 11 | Side | AACGACAATAGCCTTTCCCC |
| | | (SEQ ID NO: 37) |
| Postn Peak 11 | Side | TACTTTTGGATACACCCACC |
| | | (SEQ ID NO: 38) |
| Postn Peak 19 | Side | AGGGCAACGCTTGTTAAGAT |
| | | (SEQ ID NO: 39) |
| Postn Peak 19 | Side | CTAAAGTTTGAAATCCAACG |
| | | (SEQ ID NO: 40) |
| Meox1 Peak 5 | Center | CACAGGATATGGAGTCCGTG |
| | | (SEQ ID NO: 41) |
| Meox1 Peak 5 | Side | GAGCGTAGCCAAAATTCTGT |
| | | (SEQIDNO: 42) |
| Meox1 Peak 5 | Side | ACCCTACTAGGACATGGCAA |
| | | (SEQ IDNO: 43) |
| Meox1 Peak 9 | Center | AGCTTGCGAGAAATTCACGT |
| | | (SEQ ID NO: 44) |
| Meox1 Peak 9 | Side | TGCCTAGCTTAGTAGAAGGC |
| | | (SEQ ID NO: 45) |
| Meox1 Peak 9 | Side | CATACGCAGCTCTGTCCACT |
| | | (SEQIDNO: 46) |
| Meox1 Peak 13 | Center | GTCATTATCACGCCTCCCCG |
| | | (SEQ ID NO: 47) |
| Meox1 Peak 13 | Side | TGAACCAAGACTCCGACGGT |
| | | (SEQ ID NO: 48) |
| Meox1 Peak 13 | Side | CGTGGTTTGTAACCTCTGAA |
| | | (SEQ ID NO: 49) |

TABLE 5
Primers for ChIP qPCR

| Gene | Forward sequence | Reverse sequence |
|---|---|---|
| Postn | CAGATGAGCC | CCAGGCAGAT |
| Peak 11 | ACAAGAGGT | GACAGTCAGA |
| ChIP | G (SEQ ID NO: 50) | (SEQ ID NO: 51) |
| qPCR | | |
| Postn | ATTAAGCTGC | TTGTGGTTTGG |
| Peak 10 | CGAGCTCTTG | TATGAGACAG |
| ChIP | (SEQ ID NO: 52) | C(SEQ ID NO: 53) |
| qPCR | | |
| Postn | GAGCACAGGCC | AACAGCAGCAGC |
| Promoter | AGATCTCTT | AGAGCATA |
| ChIP | (SEQ ID NO: 54) | (SEQ ID NO: 55) |
| qPCR | | |
| Meox1 | TAGGGACAGCT | GCCTCCTCCAG |
| Peak 9 | GGGATTGTC | CCTTCTACT |
| ChIP | (SEQ ID NO: 56) | (SEQ ID NO: 57) |
| qPCR | | |
| Meox1 | TGAGCGAACA | TGGAATCCGA |
| Peak 10 | CAAACAGAG | CAGGAAAAAG |
| ChIP | G (SEQ ID NO: 58) | (SEQ ID NO: 59) |
| qPCR | | |
| Meox1 | AGTTTGCCCC | ACCTTGAGCC |
| Promoter | AGACCCTACT | AGGACCCTAT |
| ChIP | (SEQ ID NO: 60) | (SEQ ID NO: 61) |
| qPCR | | |

TABLE 6
4C primers table

| Primer | Sequence |
|---|---|
| Meox1 promoter | GCAGTGGACAGCAGATGGAT |
| 1^{st} primer | (SEQ ID NO: 62) |
| Meox1 promoter | TGCCTCAAATTCCACAAACA |
| 2^{nd} primer | (SEQ ID NO: 63) |
| Peak9 1^{st} primer | GGCTGAGAGAGGAGGGTCTT |
| | (SEQ ID NO: 64) |
| Peak9 2^{nd} primer | CAGGAGGAGGAGGGTATTGA |
| | (SEQ ID NO: 65) |

TABLE 7
Primers for detection of Meox1
enhancer peak 9 deletion table

| | | Product |
|---|---|---|
| Primer | Sequence | size |
| Meox1-Peak9- | TGTGCAAAGGACCTGGGTTT | Wildtype: |
| up-F | (SEQ ID NO: 66) | 6516 bp |
| Meox1-Peak9- | CTTGGAGGACATGGCAGGTT | Deletion: |
| down-R | (SEQ ID NO: 67) | ≈544 bp |
| ddPCR-WT-F | AAGGAACTCACCTCTGGTT | 99 bp |
| | TAG (SEQ ID NO: 68) | |
| ddPCR-WT-R | CCTTTGCCTCCCTGGAATTA | |
| | (SEQ ID NO: 69) | |
| ddPCR-WT- | TTAACACTGGGTGGTGGT | |
| probe | GATGGT* | |
| | (SEQ ID NO: 70) | |
| ddPCR-Del-F | AGGCTTTTTGAACAGCT | 169 bp |
| | TTGT (SEQ ID NO: 71) | |
| ddPCR-Del-R | CACAGACTCGCTGGACAG | |
| | (SEQ ID NO: 72) | |
| ddPCR-Del- | CTTCAGAAGCCCAAAATAT* | |
| probe | AAGCTACACCGA* | |
| | (SEQ ID NO: 73) | |

*Modified with 5′ 6-FAM/ZEN/3′ IB®FQ; ** Modified with 5′ HEX /ZEN/ 3′ B®FQ

siRNAs

| Target gene | Sigma # |
|---|---|
| Negative control | SIC001 |
| Meox1 | SASI_Mm01_00059250 |
| Brd4 | SASI_Rn01 0086984 |
| Brd3 | SASI_Rn02 00239998 |
| Brd2 | SASI_Rn01 0086984 |
| Smad2 | SASI_Mm01_0002-2387 |
| Smad3 | SASI_Mm01_0015-3031 |

### Example 2: Dynamic Reversibility of Heart Failure with BET Inhibition Tracks with Myofibroblast Cell State

This Example describes investigation of the therapeutic effects of small molecule BET bromodomain inhibition in mouse models of heart failure, and whether such heart failure is reversible upon initiation, withdrawal, and re-initiation of CPI-456 administration.

In initial studies, the compound CPI-456 was evaluated in a mouse model of heart failure induced by a permanent anterior wall myocardial infarction (MI). CPI-456 is an orally bioavailable BET bromodomain inhibitor with sub-nanomolar potency and drug-like pharmacokinetic properties, initially developed as a clinical candidate for cancer therapy.

One month of CPI-456 treatment commenced at post-MI day 5 significantly improved left ventricle (LV) systolic function (FIG. 2A). Discontinuation of CPI-456 for the next 3 weeks led to a regression of LV systolic function (FIG. 2A). Re-initiation for the next 2 weeks improved LV systolic function to the same degree as the initial treatment phase and once again, subsequent discontinuation of CPI-456 in the final week of the study led to a regression of LV function back to that of untreated controls.

Similar reversibility was observed when using the small-molecule BET-inhibitor JQ1 in a well-established mouse model of LV pressure overload induced heart failure achieved via transverse aortic constriction (TAC) (FIG. 2B). The structure of JQ1 is shown below.

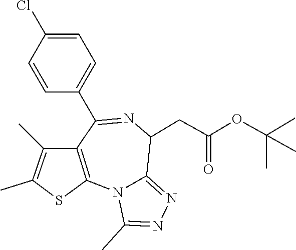 [SMILES: Cc1sc2c(c1C)C(c1ccc(Cl)cc1)=NC(CC(=O)OC(C)(C)C)c1nnc(C)n1-2]

Together, by using chemically diverse BET bromodomain inhibitors in different murine heart failure models, these studies demonstrate significant therapeutic reversibility in LV function.

As BET bromodomain inhibition reversibly disrupts enhancer-to-promoter signaling, the inventors hypothesized that exposure to BET inhibitors could drive reversible changes in cardiac cell states in vivo in a manner that correlates with their observed therapeutic efficacy.

Given the striking protective effect of BET inhibition on LV systolic function, initial experiments focused on profiling cardiomyocytes (CM). All subsequent in vivo transcriptomic and epigenomic analyses were performed in the mouse TAC model, which exerts stress on all regions of the LV in a stereotypic and highly reproducible manner. As the vast majority of adult CMs are too large to be adequately accommodated in the typical single-cell microfluidic workflow, the inventors isolated adult CMs and analyzed them by bulk RNA-Seq.

Surprisingly, the effects of JQ1 on the transcriptome of isolated adult CMs was modest when compared to the previously published transcriptomic signature of whole LV tissue (<3% overlap), strongly indicating that the most robust effects on gene expression changes were occurring in non-CM populations. Therefore, the inventors performed single cell RNA sequencing (scRNAseq) in the non-CM compartment of mouse hearts using the 10× Genomics platform.

The inventors sequenced over 35,000 individual cells collected from four experimental groups: Sham, TAC vehicle-treated (TAC), TAC JQ1-treated (TAC JQ1), and TAC JQ1-treated followed by JQ1 withdrawal (TAC JQ1 withdrawn) (FIG. 2C). Unsupervised clustering of the scRNAseq appropriately identified a diverse array of cardiac cell subpopulations, including fibroblasts (FBs), endothelial cells, myeloid cells and epicardial cells (FIG. 2D). The most striking finding in this clustering was evident in the fibroblast population, where TAC caused a large shift in cell state and JQ1 treatment lead to a dramatic reversion of this cell state to one that closely approached the Sham state (FIG. 2E). Withdrawal of JQ1 was associated with a shift of the FB population back to a TAC-like stressed state, highlighting a reversible sensitivity of this cellular compartment to JQ1 exposure (FIG. 2E). Interestingly, JQ1 exposure also led to dynamic transcriptomic shifts in the endothelial and myeloid compartments (FIG. 2E). However, in contrast to the reversible transitions of FBs between Sham- and TAC-like states, the crisp reversibility of JQ1-mediated shifts in cell state was less evident in endothelial and myeloid cells.

Given the nearly complete bi-directional reversibility of fibroblast cell states in response to JQ1 exposure/withdrawal, the inventors focused our attention on dissecting the transcriptional plasticity of the fibroblast compartment. Differential expression analysis of 13,937 individual fibroblast transcriptomes pointed to strong similarities between Sham and TAC JQ1 groups and highlighted a core signature of pro-fibrotic genes highly attenuated by BET inhibition (FIG. 2F). Sub-setting and re-clustering of fibroblasts further illustrated the robust reversibility of fibroblasts between Sham and TAC states in response to JQ1 exposure (FIG. 2G).

Cardiac stress can trigger the transition of resident fibroblasts into a contractile and synthetic state called the myofibroblast (myoFB). Overlay of the myoFB marker gene Postn demonstrated that TAC leads to myoFB activation. However, administration of JQ1 shifted the myoFB cell state back toward a Sham-like state, while withdrawal of JQ1 reverts these cells back to myoFBs (FIG. 2H). Sub-clustering of fibroblasts showed that there were 10 clusters exhibiting demarcation of basal FB states (encompassing Sham and TAC JQ1 cells; clusters 0, 1, and 4) versus the myoFB state (encompassing TAC and TAC JQ1 withdrawn cells; clusters 2, 3, and 5) (FIG. 2I).

Gene ontology (GO) analysis highlighted how gene-programs associated with basal fibroblast homeostasis were enriched in Sham and TAC JQ1 cells, while the TAC and TAC JQ1 withdrawn populations were enriched for pro-fibrotic, secretory, proliferative, and migratory gene programs (FIG. 2I). Together, these data demonstrate that the reversible transition between the basal fibroblast and activated myoFB states can be robustly toggled using transcriptional inhibition, indicating that BET protein function in myoFBs influence the trajectory of heart failure pathogenesis. Given the dynamic regulation of fibrosis-inducing and secretory proteins in fibroblasts the effects of BET inhibition may be cell autonomous and non-cell autonomous.

### Example 3: Chromatin Accessibility and Enhancer Activation in Heart Failure

The inventors hypothesized that the observed transcriptional reversibility that results from JQ1 exposure would be supported by corresponding changes in chromatin accessibility and enhancer activation in cardiac fibroblasts and other endogenous cardiac cell types during heart failure pathogenesis. To test this, the inventors integrated the single cell transcriptomic analysis with single cell Assay for Transposase-Accessible Chromatin sequencing (scATACseq) from the same hearts used for scRNAseq (FIG. 2C).

The inventors identified 490,020 accessible sites distributed among 31,766 individual cells and assigned cellular identity based on chromatin signature. The focus of this study was to dissect distal regulatory elements. The inventors therefore excluded accessible sites in promoters and gene bodies and defined a catalog of fibroblast-, myeloid- and endothelial-enriched distal elements that were used for all subsequent analyses.

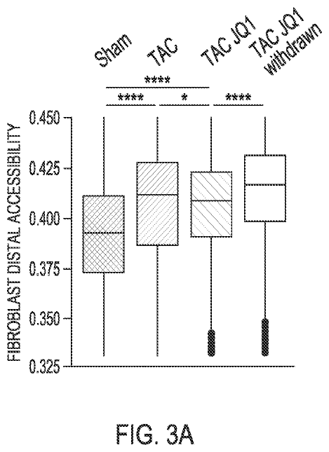

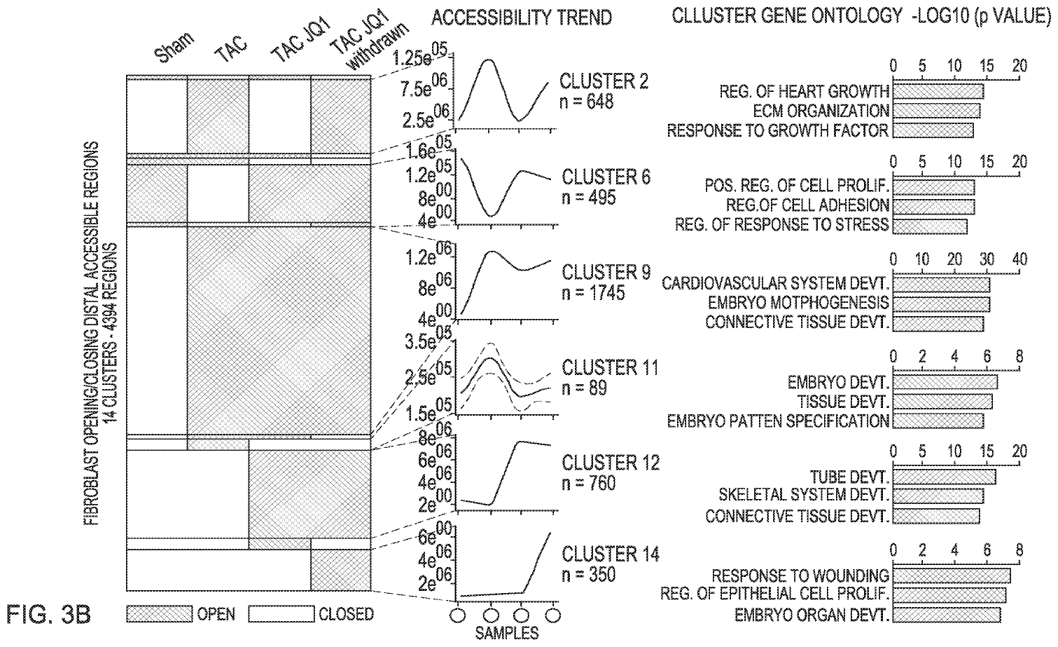

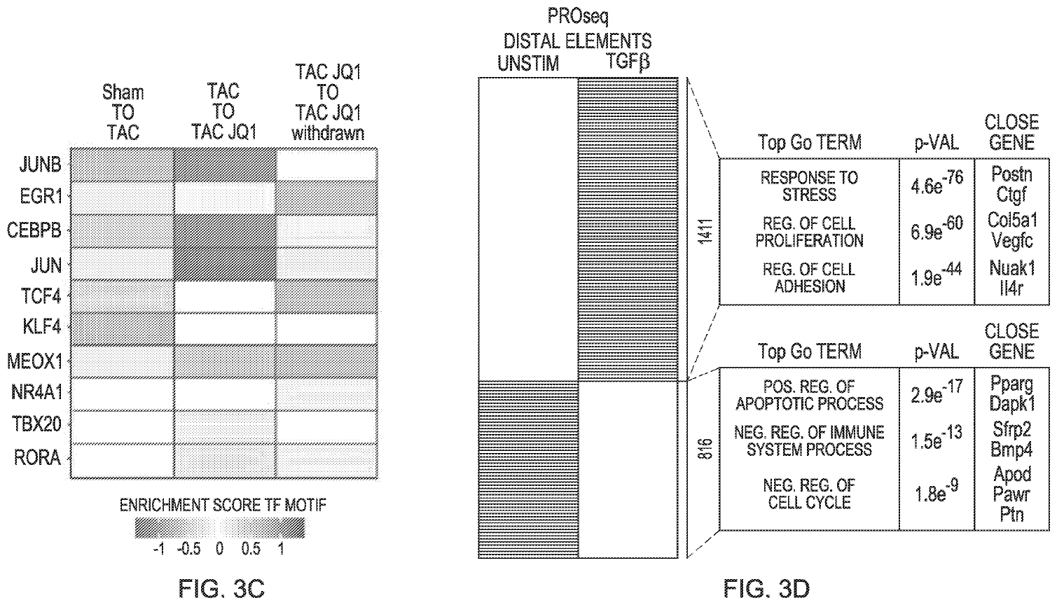

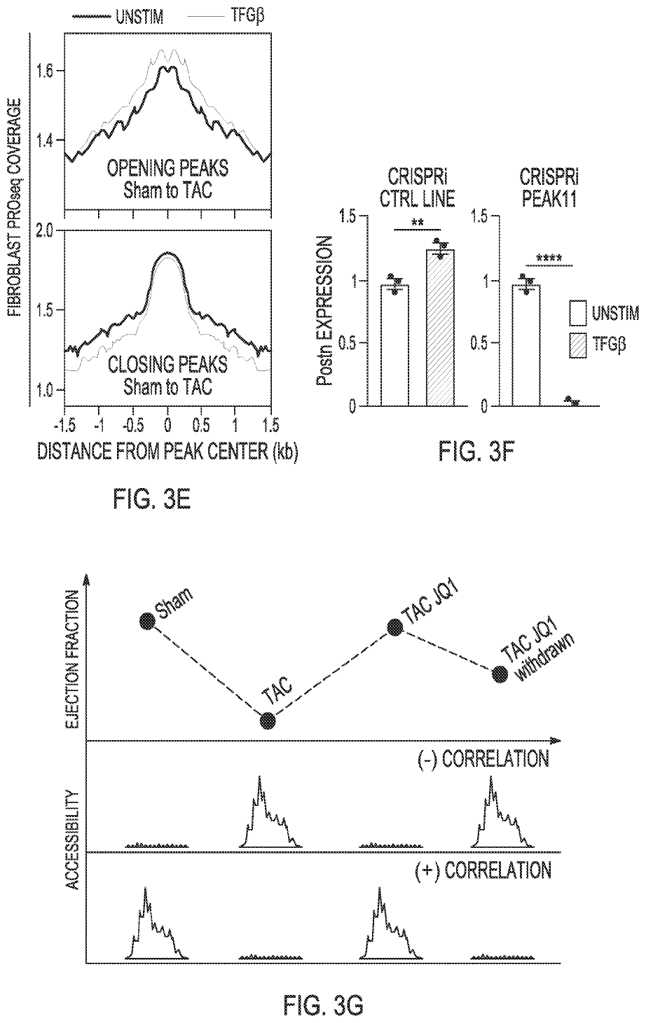

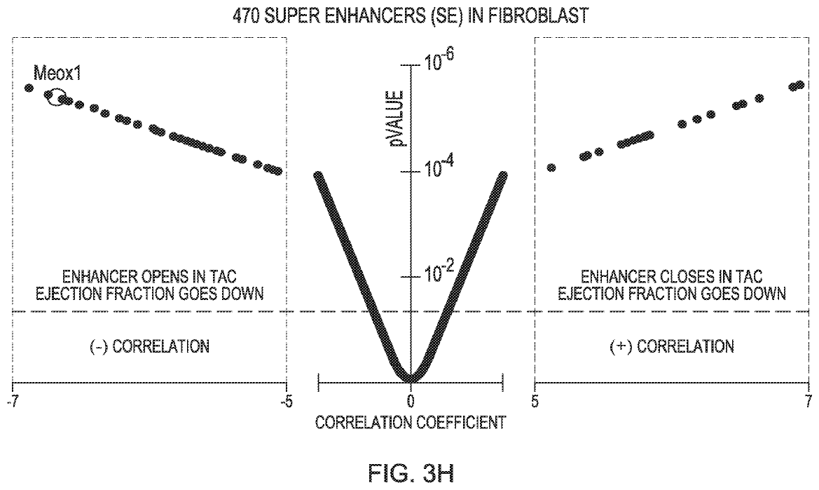

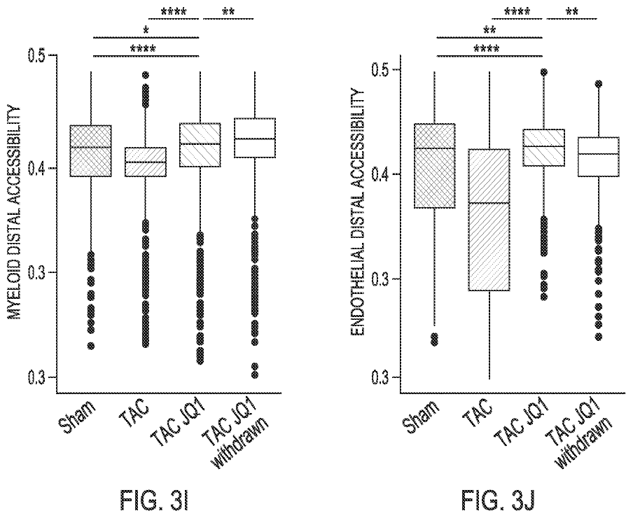

Interestingly, fibroblasts showed a significantly greater increase in chromatin accessibility after TAC that was reversibly attenuated with JQ1 treatment (FIG. 3A), a feature that was less evident in myeloid and endothelial cells (FIG. 3I-3J). This highlights how the fibroblast cell population preferentially undergoes chromatin activation during chronic heart failure that is partially dependent on BET proteins. In order to dissect dynamic and reversible changes in chromatin activation, the inventors defined open and closed distal elements across four of their samples and excluded the regions that were constitutively open across all conditions. As shown in FIG. 3B, robust reversibility of chromatin states occurred in response to stress and BET inhibition, particularly in fibroblasts.

A cluster of very sensitive and highly dynamic fibroblast distal elements was identified by the inventors that were closed in Sham, opened in TAC, closed by JQ1, and robustly re-accessible following JQ1 withdrawal (Cluster 2, FIG. 3B). GO analysis showed that these regions were in proximity of genes controlling heart growth and extracellular matrix (ECM) organization, two hallmark features of adverse cardiac remodeling and fibrosis. Interestingly, the inventors also identified a large cluster of fibroblast regions that opened from Sham to TAC that were insensitive to JQ1, highlighting a signature of stress-responsive chromatin activation that is BET-independent (Cluster 9, FIG. 3B).

Next the inventors explored how transcription factor (TF) binding motif accessibility changed in regions that were dynamically modulated in the three phenotypic transitions where significant changes in heart function occur: Sham to TAC, TAC to TAC-JQ1, and TAC-JQ1 to TAC-JQ1 withdrawn. In fibroblasts, TF binding motifs for CEBPB, JUN and MEOX1 showed enrichment in accessible regions in the Sham to TAC transition followed by loss of enrichment with BET inhibition that was then re-acquired with JQ1 withdrawal. These data indicate that chromatin dynamics occur at regions enriched with functionally relevant motifs for stress-activated TFs (FIG. 3C).

The inventors then sought to identify functionally relevant fibroblast and activated myoFB enhancers discovered during scATACseq. Studies indicate that enhancers can be pervasively transcribed and that this nascent transcriptional activity is a robust and independent indicator of enhancer activity. Hence, the inventors performed precision nuclear run-on sequencing (PROseq; Mahat et al., Nat. Protoc. 11: 1455-1476 (2016)) on cultured fibroblasts in vitro to map genome-wide RNA polymerase II nascent transcription and identify putatively active enhancers. Because PROseq requires large quantities of cells, the inventors generated an immortalized line from primary adult mouse cardiac fibroblasts and treated the immortalized cell line with TGF-β, a canonical stimulant for eliciting myoFB cell state transition in vitro (Dobaczewski et al. J. Mol. Cell Cardiol. 51, 600-606 (2011)). A set of distal elements were identified by the inventors that were significantly more transcribed after TGF-β stimulation and were located close to pro-fibrotic and pro-synthetic genes (FIG. 3D). Using the scATACseq data, the inventors identified the distal elements that were either opening or closing between Sham and TAC in vivo, and then assessed PROseq signals in the cultured FBs at these same regions. As shown FIG. 3E, in vitro TGF-β stimulation of cultured fibroblasts triggers global transcriptional changes that resemble those that occur in endogenous fibroblasts in vivo during heart failure pathogenesis. Visualization of the Postn locus illustrated where there is chromatin opening in vivo occurred after TAC and that was correlated with dynamic sensitivity to JQ1 exposure. Within this large enhancer, PROseq revealed a specific region—Peak 11—that was heavily transcribed following TGF-β treatment (Peak 11). scATACseq co-accessibility analysis between the Postn promoter and the Peak 11 region showed low co-accessibility in the Sham state, a robust increase in co-accessibility in response to TAC, and modulation of co-accessibility in response to JQ1 exposure.

CRISPR interference (CRISPRi) deploying a catalytically inactive Cas9protein (dCas9) fused to the KRAB repressor protein (Gilbert et al. Cell 154, 442-451 (2013)) was used with a guide RNA specific to the PeakI 1 region to drive sequence-specific repression of this particular regulatory element in fibroblasts. The inventors found that this Peak 11 region is essential for Postn induction following TGF-β stimulation (FIG. 3F).

Having demonstrated the dynamic transcriptional control of Postn, a marker of myoFBs, the inventors hypothesized that the integrated single cell transcriptomic and epigenomic approach described herein could be leveraged to discover novel mechanisms controlling cellular stress responses during disease pathogenesis. Hence, the inventors built an unbiased enhancer discovery pipeline to unveil distal elements that could play a role in the progression and reversal of heart failure. A catalog of cell population-enriched large enhancers (also known as stretch- or super-enhancers) was assembled for fibroblasts, myeloid and endothelial cells using our scATACseq data in the diseased heart (TAC). As BET inhibition robustly improved heart function in the TAC model, the inventors correlated the degree of accessibility of these enhancers in fibroblasts, myeloid and endothelial cells with LV ejection fraction. This correlation analysis between enhancer chromatin accessibility and a physiological trait (in this case LV ejection fraction) is summarized in FIG. 3G. Enhancer elements were defined as having a negative correlation if their accessibility was anti-correlated with heart function (i.e., these enhancers were opening from Sham to TAC, a setting where cardiac function decreases). Conversely, enhancers with a positive correlation were those that closed from Sham to TAC. A Volcano plot of correlation coefficients was generated for each cell type (FIG. 3H). Of the 470 large enhancers identified in fibroblasts, forty-eight showed a strong negative correlation while twenty-two showed a strong positive correlation (FIG. 3H).

### Example 4: Meox1 is a Myofibroblast-Specific Transcription Factor

This Example illustrates that Meox1 is a myofibroblast-specific transcription factor.

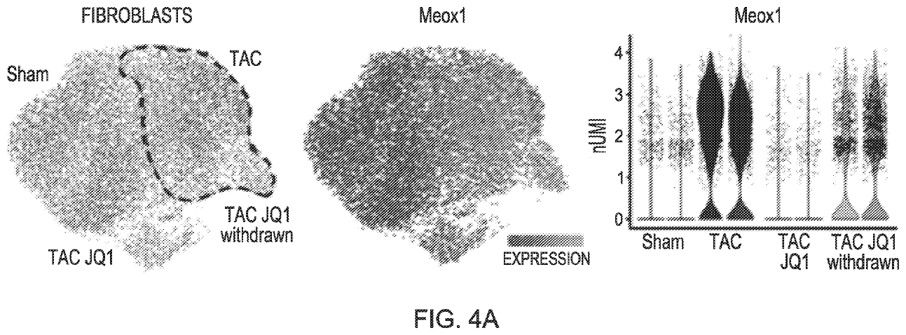

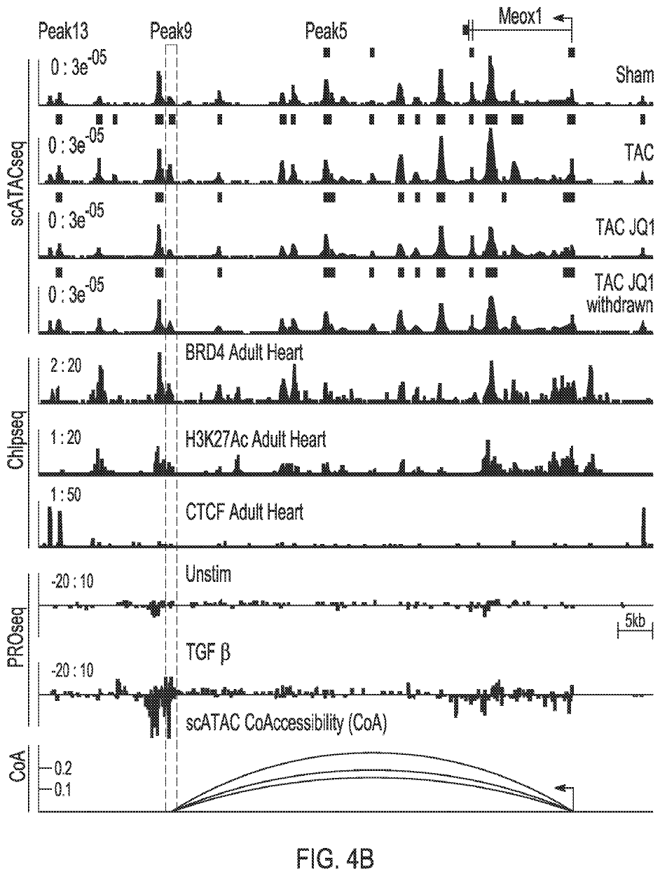

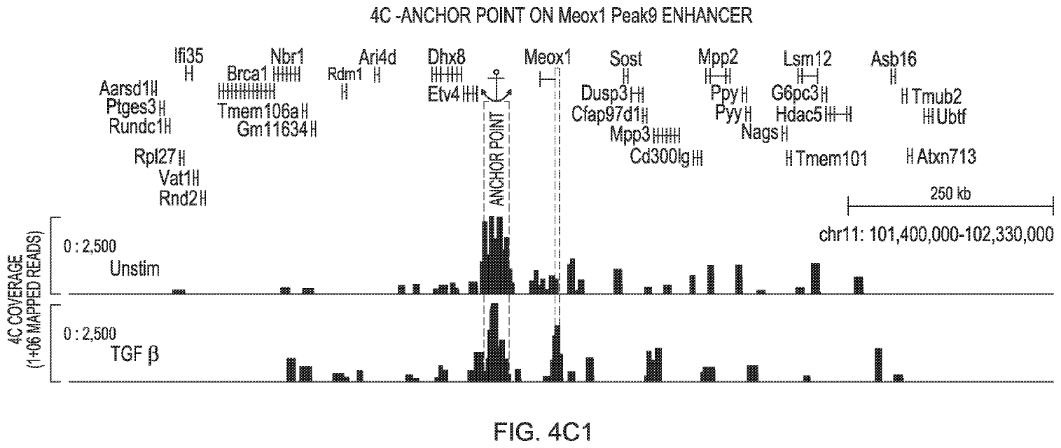

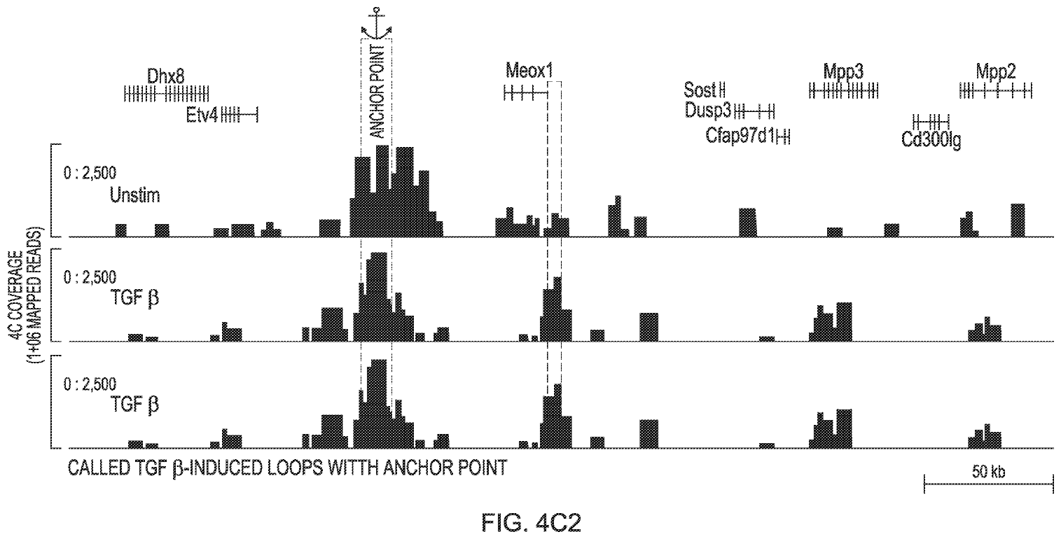

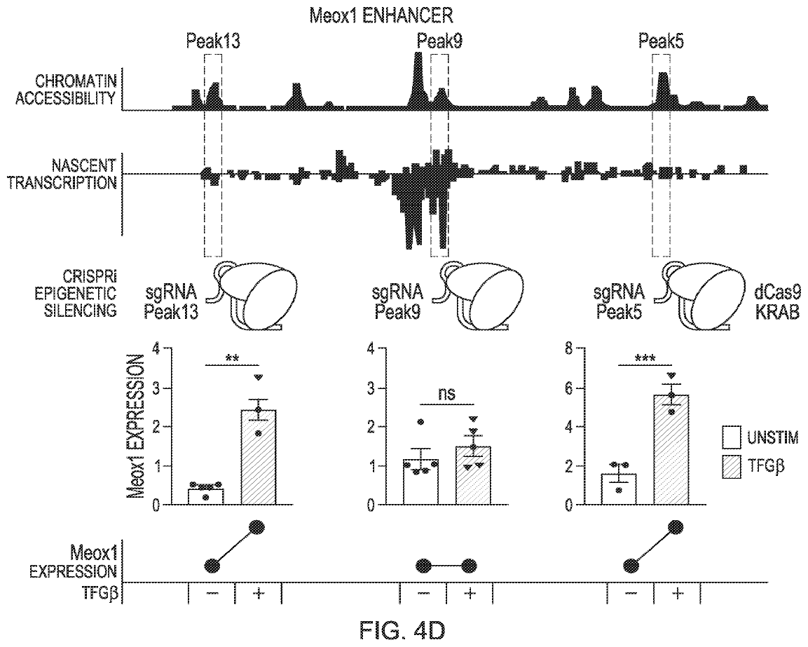

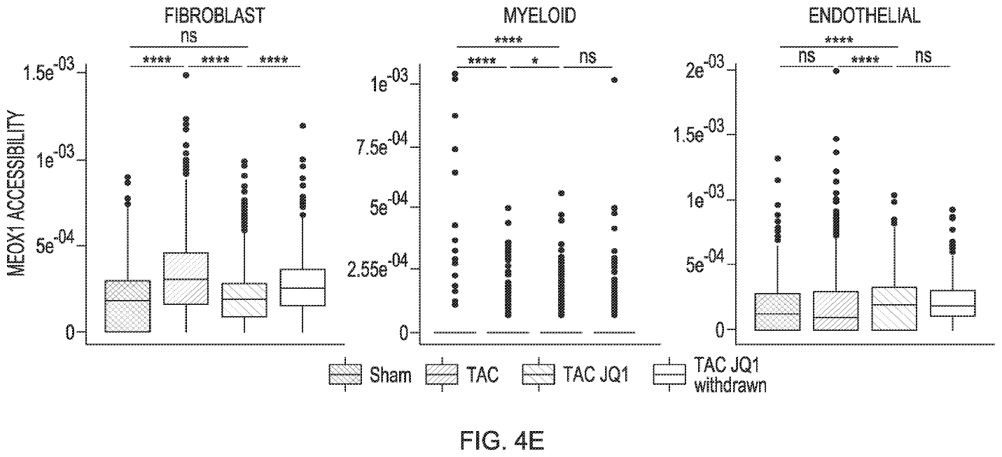

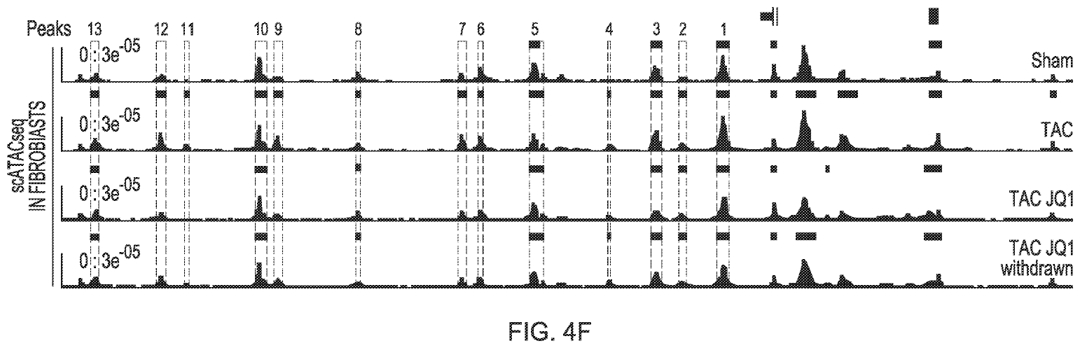

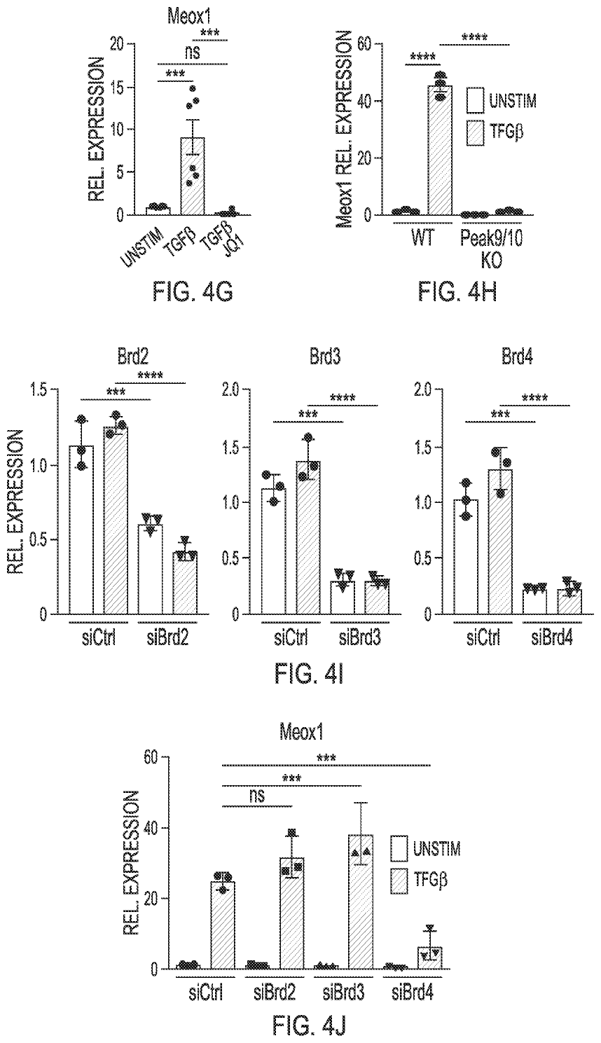

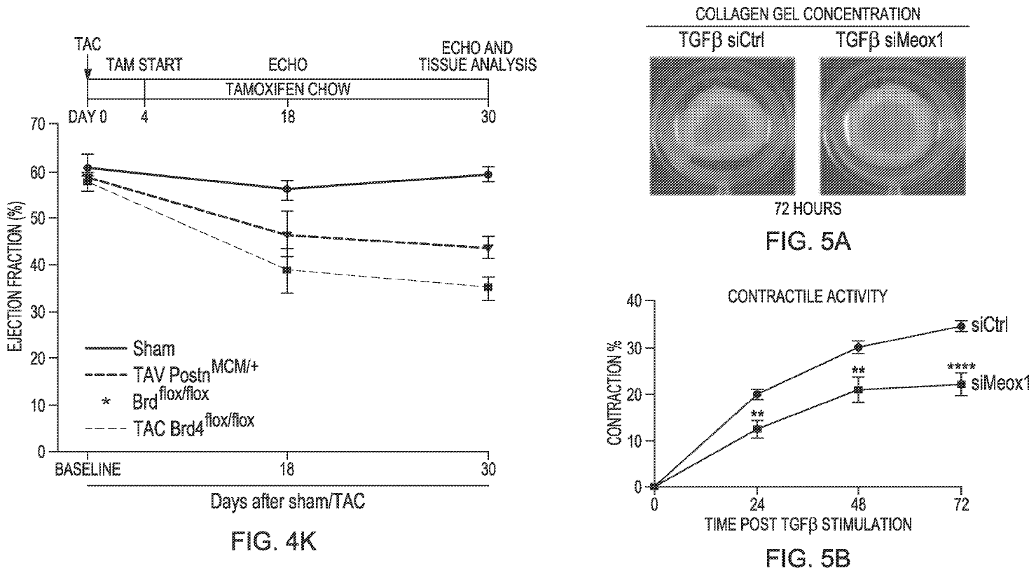

One of the most negatively correlated elements in fibroblasts was a large enhancer downstream of Meox1 (FIG. 3H), a homeodomain-containing transcription factor that is expressed in paraxial mesoderm and is required for sclerotome development. Meox1was particularly interesting because it was minimally expressed in the healthy mouse heart but highly upregulated in MyoFBs following TAC (FIG. 4A). BET inhibition abolished Meox1expression while JQ1 withdrawal was associated with its robust re-induction (FIG. 4A). This, combined with the corresponding enrichment of the MEOX1 DNA-binding motif in dynamically accessible regions of chromatin in endogenous cardiac fibroblasts (FIG. 3C), indicated dynamic upregulation and functional engagement of MEOX1 with key fibroblast regulatory elements under stress conditions.

The enhancer downstream of Meox1 was extremely sensitive to stress and JQ1 exposure in fibroblasts, but not in myeloid and endothelial cells (FIGS. 4B and 4E). The enhancer had 10 peaks that significantly opened from Sham to TAC conditions in fibroblasts such that the peaks became accessible in this transition, closed with JQ1 treatment back to a Sham level, and re-opened when JQ1 was withdrawn (FIG. 4B, 4E-4F). Analysis of the chromatin accessibility in all individual peaks of the Meox1enhancer showed that particular elements were dynamically modulated.

A sequence for a Mouse Meox1 Peak9/10 region on mouse chromosome 11 at positions 101,828,401 to 101,833,068 is shown below (SEQ ID NO: 74).

```
      1 GCATTAATTT TGAGTTTCCA AAATTCACAT TAGATAGTGT
    41 ATAGCTTGTA TTCTGAGCTT GAGGCAAGAC ACCCTTTCCA
    81 CTTTGTCTGG AGCGAGTGGC TCACTGGTAT CTTAGAGTCT
  121 TTGCTCAGTG TATGAGAAAC ACTGAGTCCC AGAATGAGCA
  161 CCTGCAAGCC CTTCTATTCA TCTATTAGCC CAGGGTGACC
  201 TTGAGCTTGT GATCCTGTTG CCTCAACACC TGAATGCTGG
  241 TTTTATGGGA CACACCACCA CACCTAAGTC CAGGACTGGG
  281 ACACTGAATG TCTTCTAAGC CTTCTTCTCT TCTAGCTACC
  321 AGCCCCTCCC CTGCCGGTGT TGCAGAATAT CTGATAAAAG
  361 TTAACACCCT TGCCTGGCTC AGCAGCTCCC ACTCTCTCTC
  401 TCCTGATTGA TCCCCCACCT CCAACCCTAA TCATTGCACC
  441 CCAACTTCCA CAATGTCACC CAGGGCACCT GCACATCTCA
  481 TCTCCCTGGC CCTTGACTAA ACTTTCCAGG GTCCTCAGCA
  521 CAAACTCCCT GTTGCCTGAA ATTACATCAG CTTTTTGACG
  561 AAACTTGAGT CAGATGTGTG TGTGTGTGTG TGAAATAATT
  601 CTCTCTTTTA AAAAATATCC ATTTATTTTT ATCTTGTGTA
  641 CATGAATGTT TTGTCTGCAT GCACGTCTGT GTATCACGTG
  681 TGTGCAGTAC CTGTAGAGGC CTGAAGAGAT TGTGAGCCAC
  721 TATGTGGGTG CTGGGAACCA AACCTGGGCT GTTAGGAAGA
  761 ATAACCACGG AGTCACCTCT CCAGCCCAGA AATGCTATTT
  801 TCTCTCTCTC TTTTAGAAAA CAGAACAAAG AGTCTAGCAG
  841 AGCAGGCCTA GCCCTCAGAT CCTCTGCCCC AGTGCTAGCA
  881 GACTCCCCAT GGTGTGGGTG CATGTATATC CCTTCTCTTC
  921 CCTGCTTCTC TCTTCGCTCT GGGTCCATTC CTCTACACCC
  961 ACCCACACCC CCTGACTTGC CTCTTTTATT TTAAAACATT
1001 AGTTTGTCTA TTTGATGTTT GTCTGTAATT GTGTGTGCTT
1041 GAATGCAGAG CTCTGAGGGC AACTTGTGGA GTCAGATCTC
1081 TCCGTCTGTC ATGTGGGTTC TGAGTTTAGA ACTCAGGCTA
1121 TTAGCTTTGG CCGCAAGCCC TTTCTCCCAC TGAGCCATTT
1161 TAGTAGCCCT TACCTGCCTT TTGAAGCAGC GTCAATCTCT
1201 TCTGTGCCTG GTGCCTGATC CAAGGCCCGC CCCCCCAAAC
1241 CCCCATTTTG GTTCTTTATT TACTGGGCTC TATGAACCTT
1281 GAGGGTAGGT AACACCTTTC ATATCTCCTC TGTCTCTAGT
1321 CCCACACCCT CCACACAGGG TCAGGCACAA AGTAGGCATG
1361 AATGAGTGAA TGAGTGAATG AATGAATGAA CGAAATAAAC
1401 ACTGCAGCAG AAAAAAATCT TTTCCTTATT GTTTCTTGGC
1441 TAGATCTGGG GCAGAGCTCT GTGACCCCCC TAGCCTCAAA
1481 CCAGAGCTCG GAACATCAGG TTTGGAGGTC CAGGTTCCTG
1521 GTTCCCCTAG CCCAAAGAGG ACAATAAAGG CCTCCTCTGG
1561 AGAGCCGGGT CACTCCACCG GAGGACATTA AAACAAAACT
1601 GAATTAACTA CTCTTCACAT GGAACCCAGC TTCGTGAGTG
1641 TGCTAAGTGT CTTATTAAAC AAACAACCCT AAAAGATAGA
1681 CGCGCCCCGC TATCGTTTCT CATAGTAAGA GAAAAACCAA
1721 AGGCCCAGGG CTGGGGGCTT TGGGGGAAAC CTGGCTCTTA
1761 TTCTTTGGGG CCGGAGGTCA CTGGAACAGC TGCTGCCTGG
1801 GGATCAAGCT CAAGGTTCTG GAGAGATGGA AAAACCTGGC
1841 CTGCCGCTTT TCCTCCGCGG CCCCTCAGCT GCTAACAGGA
1881 GCGAGCTGAG CGAACAGAAA CAGAGGCGCT GTGACCGGCC
1921 CAGATCCGAG CTTCCTCTGA AATTTCCAGC CTCCTTTTTC
1961 CTGTCGGATT CCAGACAGAT GAAACTTTCC TGGCCCTGGC
2001 CTGTTCCTGG CAGCATCTCT CCTCTGAATC ACCATAAATC
2041 AGGTCTGGGG GTGAGCAGTG GTTTTTTTTC CTGATTGACA
2081 AGCTGCTGCC TCGGTATAGC AGGATCTCGG CTTCATCGGC
2121 TAATAAAATA GGTCGGGGTG GGGTGGGGGG GGGGGGACGC
2161 GGGAGTGTTG ACTAAGCCCT CCCCGACACA AGTCTGTTTA
2201 GGTATCCCTT TGTATTTGAA AGTTTTAAAT CTGTCTATGA
2241 TTATTATTTC ATTGAGAAGG GCTGCGGATA TATAGATATG
2281 GGCCTTTGGG GGGCTGCGGT GGTCGTAAGC AACACCATTG
2321 TTTGAGGGAT CACATAGGAG GCCTGTACAT TCCTTAAAGT
2361 CTCCATCATC CATGAGTCCC CAATTAATTT CAATACAAAC
2401 ACCGGATCTG GCCTCCTCTG TGTCTCTGTC TCTGCAGCCA
2441 TAACACATGA GGTCATGCAG TTTTGGTTAA CCAACCGGAG
2481 GTGCCTGATG GAGGTGGGGG GCTTCCAGCC CTCGGGAGGC
2521 TGGGGTATCT GCCCTGCCTG ACTCCAGCAG GGCCGTTTAT
2561 TGAAGCGAGG GGTCATGGGC ACCCATACTC ACTTCTTTGA
2601 TGGCTTTTCC TCACCCCTGC CCCCTTCATC TCAAGCCACC
2641 CATCTGTCAT AGTGCCACCC TGTGTTTTTG TTGTTTGTTT
2681 TGIAACAGGG TCTTGGAGTC TCCCAGGCTA GTCAAGGAAG
2721 GCCTTGTACT TCTGATCTTC CTGTC7TCTG TCCCTGGTTT
2761 TATGAAATGG GGGATTGAAT TTAGGGCTTC ATGCATGTCA
2801 GGCAAGCTAC ATGCTACCTC TTTTCCCCTT TAGTTTTAGG
2841 TAGGGACAGG GTCTCATGTA TCCAAGAATG GGAATGGCTT
2881 CAGACTTGCT GTGTAGCTGA AGATGATCTT GAACTCTTGA
2921 TCCTGCTGTG TCCATCTCTT GCGTGCTGGG ATTATAGGCA
2961 TGTACCACGC TACACCTGGT TGGCACTCCT GGGATCAAAC
3001 CCAGTGCTCT GTTCAATCTA CCCTCCCATC CCCCCTTCAC
3041 CATCCCCTCT CCCCCTCCCC TCCCTCCTTT GCCCCTCTCT
3081 CCCATCCCTC TTCCTACCTT CCCCCTCTGT CCCCTCCCCT
3121 TCCTCCCCTT CCTTCCTTCC CTCTCCTCTC CCTCCTCCTG
3161 CCTACCATCC CCTCCCTCCC ACTCCTTACC ATCCCTCTCC
3201 CTAACAAACC CTTCCCTCAC TTTTTTCTTT TTGACCCCTC
3241 CCCTAATCTT CCCTTTTAAG ATAGGATCTC ACTGTGTAGC
3281 CTAGCCTGGT CTCAAATTCA AGACCCTCCT CTCTCAGCCT
3321 CCTGGGTGCT GGGGTACAGG CATACACCAT TACAGGCTTC
3361 CACCCTGTGG TTCTTTAAAC TCTCCTCCCC TTTAGTACTC
3401 TTTGCATACG CAGCTCTGTC CACTTGGAGT GTCTTGTGGC
3441 CTTTTAATCT GTCTCCTTGT TTACAGCAG3 AGCCTTCTTC
3481 CTCCAGGAAG GCTTTCTGCC TCATTTCATC CGGGGTTAGT
3521 TCCTGCTTCT ACACAGCCTG TTGGTTCTCA GACAGCCATT
3561 CACATCAGAA TCACCTGGAG GGTTTATGAA AGTGTTGACT
3601 GGTAGCCTGC TCCCACATTC TGCATTATCT GACTTGGACT
3641 GGGCTGGGCC TGCCAACGTG AATTTCTCGC AAGCTCTCAG
3681 GGGACTCCGA TGCTACCACA GCCTCTGGCG TCATCCTCCT
3721 TGATATCATT TATCCTGCTG GCTTGCTACT ACTCACTTAT
3761 TTGTGTGCCT CTCCCTAGGG ACAGCTGGGA TTGTCTTTTT
3801 CCTCTCTAAG CCTATGGTTT GGACAGCTGT GTGCACAGTG
3841 AAGCTGCTCT CGCTGGAGGT TTGCTGATTA AATGAGTTGT
3881 AGATTCAGAG GAGGCTGGCT GACTGCCTAG CTTAGTAGAA
3921 GGCTGGAGGA GGCGCCTATC TCCCAGAGGA GGCTGTTGGG
3961 CAGGAGGGCT GGTTTTCGGG GACCACAGGA TGGTGAGTGG
4001 GGATTGTCTT CATCAAGGGA AATGAGCACA ACTCTCCCCT
4041 TCTGTGCACA TCTCTAGGAA GAGATGGGAG GCCAGTGGTT
4081 CAGGTGCCAA CAGGAGGAGG AGGGTATTGA ACTGTCTTAG
4121 TTAAGGTTTT ACTGCTGTGA ACAGACACCA TGACCAAGGC
4161 AACTCTTATA AGGGCAACAT TTAATTAGGG CTGGCTTACA
4201 GGTTCAGAGG TTCAGCCCAG TATCATCAAG GTGGGAACCT
4241 GGCAGCATCC AGGGCAGGCA TGGTGCAGGA AGAGCTGAGA
4281 GTTCTACATC TACATCTGAA GGCTGCTAGC AGAATACTGG
4321 CTTCCAGGCA GCTAGGATGA GGGCCTTAAA GCCCACACCC
4361 ACAGTGACAC ACCTACTCCA ACAGAGCCAC ACCTTGTAAT
4401 AAAGCCACTC CCTGGACTGA GCATATACAA ACCATCACAT
4441 GAAATCCACC TGATTTTTCT CTCTGAGACC TGGGAAGGCT
4481 GGATGGTGAA AGAATACATG GCTGTTCTTT TCGTTCCATG
4521 GTGTGTGTGT GTATGTACAT GCACATGTGT GTGTGCATAT
4561 GTGTAGGCCA GAAGTCAATG GTGTGTGTTT TAATAATTCT
4601 CTATCTTTTA TTTTCGGAGG CAAGGTCTCT TGTTTAATGT
4641 GGAGCTCACA GAGTCAGCCA TACTTGCT
```

Publicly available BRD4 and H3K27ac ChIP-Seqdata from adult mouse LV tissue corroborated the active enhancer marks at the Meox1 locus, and CTCF ChIPseq was consistent with the absence of contact insulation between the Meox1gene and the enhancer, raising the possibility that this enhancer regulates Meox1 (FIG. 4B). Importantly, Meox1 mRNA expression was induced in cultured fibroblasts treated with TGF-β (FIG. 4G).

TGF-β-induced Meox1 upregulation was suppressed by JQ1 and knockdown of each of three BETs (Brd2, Brd3 or Brd4) individually with siRNAs demonstrated that Meox1 induction was dependent on BRD4, but not BRD2 or BRD3 (FIG. 4I-4J). Among the individual scATAC-Seq peaks in this locus that showed increased accessibility with TAC in vivo, PROseq of cultured fibroblasts was able to identify a specific region located 62 kilobases (kb) downstream of the Meox1promoter (Peak 9/10) that featured a striking increase in nascent transcription following TGF-β stimulation (FIG. 4B). Notably, this 780-base pair (bp) element showed stronger TGF-β stimulated transcription than the Meox1 gene body itself and was also one of the most differentially transcribed regions across the whole genome in response to TGF-β stimulation (FIG. 4B). The Meox1promoter and the Peak 9/10 region showed low co-accessibility in the Sham state, a strong increase in co-accessibility in response to TAC, and modulation of co-accessibility in response to BET inhibition (FIG. 4B). Chromosome conformation capture analysis of this locus in cultured fibroblasts revealed a robust increase in contact between the Peak 9/10 enhancer region and the Meox1 promoter in response to TGF-β stimulation (FIG. 4C), consistent with dynamic contact between these elements. Compared to the other regions within the large Meox1 regulatory element, Peak 9/10 featured strong chromatin accessibility and nascent transcription, two features that are strong predictors of a functionally relevant enhancer. To definitively interrogate the endogenous function of Peak 9/10, the inventors performed a series of CRISPRi experiments in the Meox1 locus using guide strands specifically targeted to individual sites and found that the Peak 9/10 element was required for Meox1 transactivation upon TGF-β stimulation (FIG. 4H) while other accessible regions identified in vivo were not (data not shown).

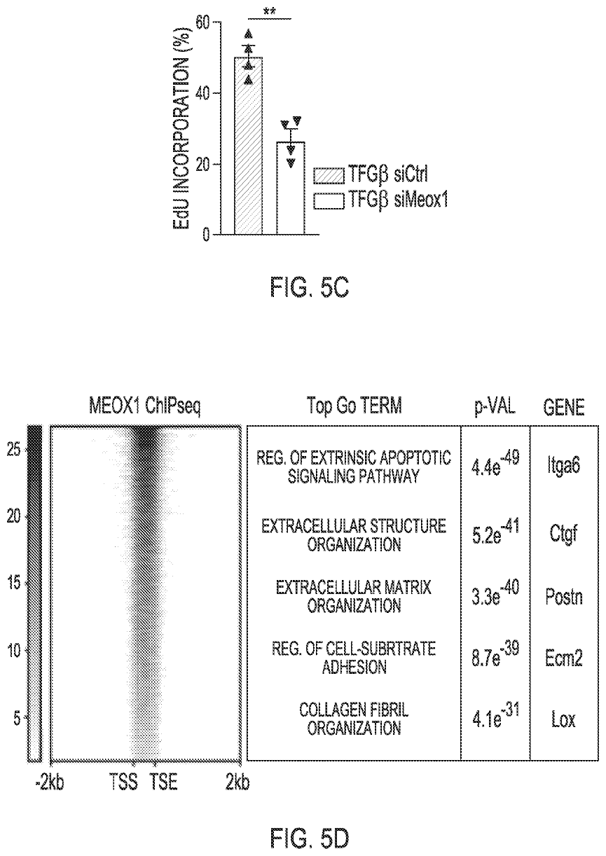

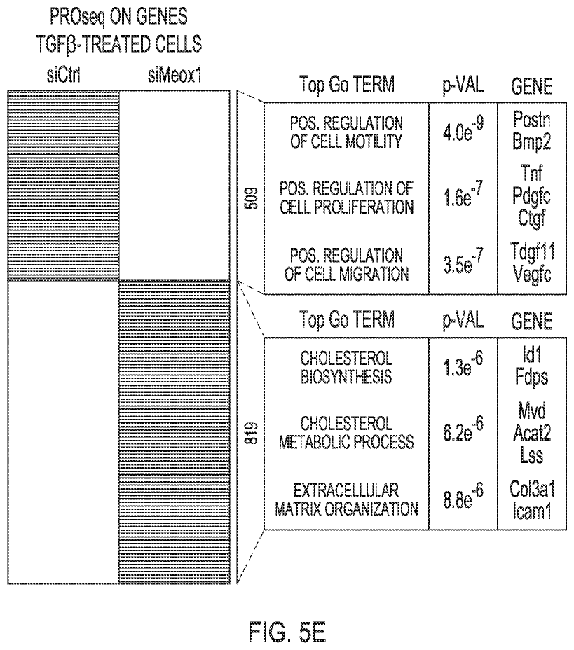

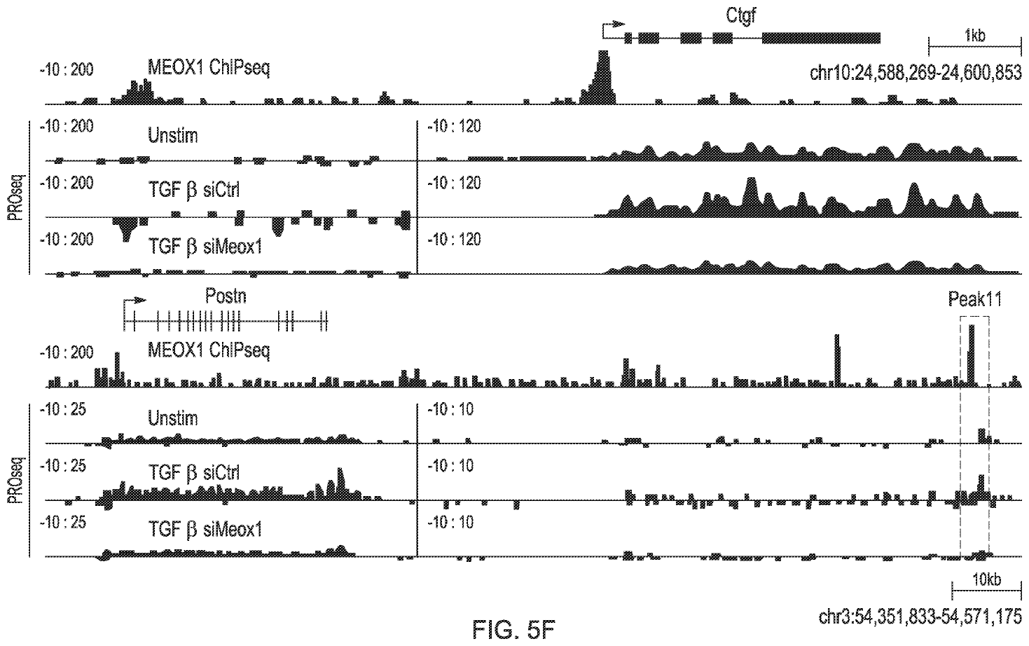

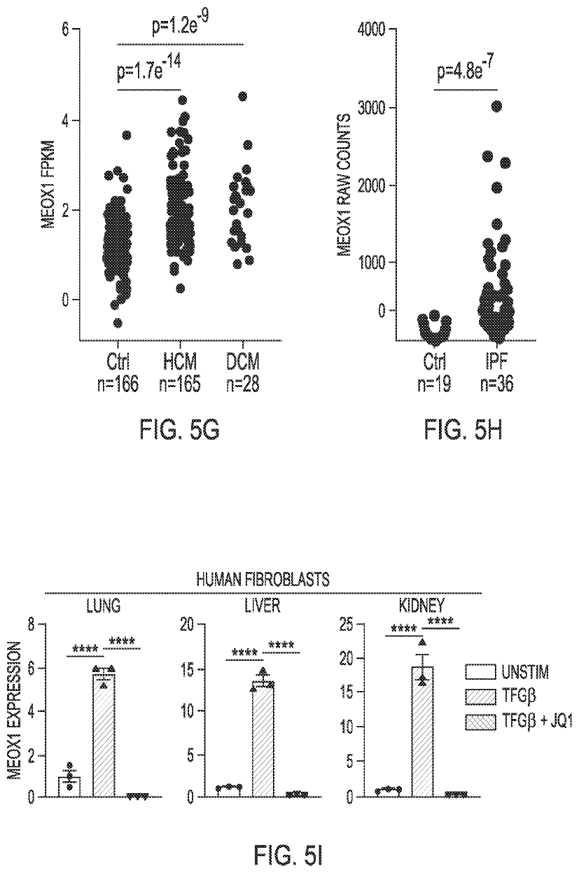

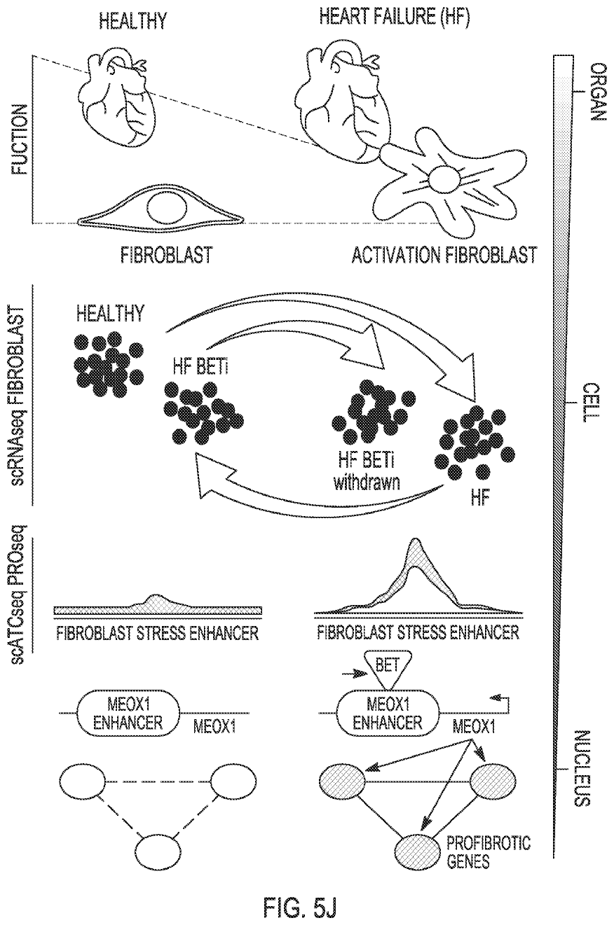

The inventors next investigated the function of MEOX1, hypothesizing that this poorly characterized homeobox transcription factor might directly regulate gene programs involved in fibrotic disease. Knockdown of Meox1 by a siRNA led to significant reduction in TGF-β-stimulated collagen-gel contraction and EdU-incorporation, confirming that MEOX1 was required for contractile and proliferative phenotypic transitions, two functional hallmarks of MyoFBs in disease pathogenesis (FIG. 5A-5C). ChIPseq showed that MEOX1 binds genes involved in fibroblast homeostasis and response to stress (FIG. 5D). GO analysis of the highly MEOX1-bound genes showed enrichment for terms linked to apoptosis, ECM/collagen organization and cell adhesion.

In order to understand whether MEOX1 controls the transcription of stress-responsive pro-fibrotic genes, the inventors performed PROseq in TGF-β treated fibroblasts in the presence of either a control or a Meox1-targeting siRNA. 509 genes were significantly less transcribed when Meox1 was depleted, while 819 were more transcribed (FIG. 5E). Notably, GO analysis of the Meox1-dependent genes revealed enrichment for pro-fibrotic processes such as regulation of cell motility, proliferation, and migration. Among these genes were classical markers of cardiac MyoFB activation, including Ctgf and Postn, which showed MEOX1 enrichment at their promoters and proximal regulatory elements (including the PostnPeak11 enhancer described in FIG. 3F), regions that featured strong decrease in transcription following Meox1depletion (FIG. 5F). These findings indicate that MEOX1 functions as an essential transcriptional mediator of the fibroblast to myoFB switch associated with fibrotic disease. Ysing recently publicly available single cell data from the human adult heart (see, heartcellatlas.org), the inventors found that MEOX1 was specifically expressed in the same subset of activated fibroblasts as POSTN.

The inventors next explored whether MEOX1 activation during fibrotic disease was conserved from rodents to humans. Single cell data from the human adult heart indicated that MEOX1 was expressed in activated fibroblasts and together with POSTN was one of the top genes determining the cluster of activated fibroblasts. A recent atlas of chromatin accessibility from the human fetal heart indicated that the syntenic region of Peak9/10 was characterized, by the strongest signal of accessible chromatin in the MEOX1 distal element in fibroblasts. Like heart failure, many grievous human diseases feature maladaptive fibroblast activation.

As fibroblasts can have tissue-specific behaviors, the inventors investigated whether MEOX1 was also induced in fibroblasts derived from human lung, liver and kidney, three organs that often develop substantial fibrosis in the setting of chronic organ dysfunction. Similar to our findings in the heart, MEOX1 expression was induced by TGFβ and suppressed by JQ1 in fibroblasts from human lung, liver and kidney (FIG. 5I). Furthermore, MEOX1 expression was significantly up-regulated in heart tissue from patients with cardiomyopathy (n=193) and in lung tissue from patients with idiopathic pulmonary fibrosis (n=36), two human diseases that prominently feature pathological fibrosis (FIGS. 5G-5H).

Notably, MEOX1expression was significantly up-regulated in human diseases that prominently feature fibrosis, such as heart tissue from patients with cardiomyopathy and lung tissue from patients with idiopathic pulmonary fibrosis (FIG. 5G-5J).

### REFERENCES

1. Filippakopoulos, P. et al. Selective inhibition of BET bromodomains. Nature 468, 1067-1073 (2010).
2. Anand, P. et al. BET bromodomains mediate transcriptional pause release in heart failure. Cell 154, 569-582 (2013).
3. Valente, M. et al. Optimized Heart Sampling and Systematic Evaluation of Cardiac Therapies in Mouse Models of Ischemic Injury: Assessment of Cardiac Remodeling and Semi-Automated Quantification of Myocardial Infarct Size. Curr. Protoc. Mouse Biol. 5, 359-391 (2015).
4. Li, D., Wu, J., Bai, Y., Zhao, X. & Liu, L. Isolation and culture of adult mouse cardiomyocytes for cell signaling and in vitro cardiac hypertrophy. J. Vis. Exp. (2014). doi:10.3791/51357
5. Love, M. I., Huber, W. & Anders, S. Moderated estimation of fold change and dispersion for RNA-seq data with DESeq2. Genome Biol. 15, 550 (2014).
6. Becht, E. et al. Dimensionality reduction for visualizing single-cell data using UMAP. Nat. Biotechnol. 37, 38-44 (2018).
7. Hu, K. H. et al. ZipSeq: barcoding for real-time mapping of single cell transcriptomes. Nat. Methods 17, 833-843 (2020).
8. Przytycki, P. F. & Pollard, K. S. Semi-supervised identification of cell populations in single-cell ATAC-seq. BioRxiv (2019). doi:10.1101/847657
9. Stone, N. R. et al. Context-Specific Transcription Factor Functions Regulate Epigenomic and Transcriptional Dynamics during Cardiac Reprogramming. Cell Stem Cell 25, 87-102.e9 (2019).
10. Whyte, W. A. et al. Master transcription factors and mediator establish super-enhancers at key cell identity genes. Cell 153, 307-319 (2013).
11. Acharya, A., Baek, S. T., Banfi, S., Eskiocak, B. & Tallquist, M. D. Efficient inducible Cre-mediated recombination in Tcf21 cell lineages in the heart and kidney. Genesis 49, 870-877 (2011).
12. Kwak, H., Fuda, N. J., Core, L. J. & Lis, J. T. Precise maps of RNA polymerase reveal how promoters direct initiation and pausing. Science 339, 950-953 (2013).
13. Bolger, A. M., Lohse, M. & Usadel, B. Trimmomatic: a flexible trimmer for Illumina sequence data. Bioinformatics 30, 2114-2120 (2014).
14. Langmead, B. & Salzberg, S. L. Fast gapped-read alignment with Bowtie 2. Nat. Methods 9, 357-359 (2012).
15. Ramirez, F. et al. deepTools 2: a next generation web server for deep-sequencing data analysis. Nucleic Acids Res. 44, W160-5 (2016).
16. Guo, Y., Mahony, S. & Gifford, D. K. High resolution genome wide binding event finding and motif discovery reveals transcription factor spatial binding constraints. PLoS Comput. Biol. 8, e1002638 (2012).
17. Liao, Y., Smyth, G. K. & Shi, W. featureCounts: an efficient general purpose program for assigning sequence reads to genomic features. Bioinformatics 30, 923-930 (2014).
18. Quinlan, A. R. & Hall, I. M. BEDTools: a flexible suite of utilities for comparing genomic features. Bioinformatics 26, 841-842 (2010).
19. Stadhouders, R. et al. Multiplexed chromosome conformation capture sequencing for rapid genome-scale high-resolution detection of long-range chromatin interactions. Nat. Protoc. 8, 509-524 (2013).
20. Krijger, P. H. L., Geeven, G., Bianchi, V., Hilvering, C. R. E. & de Laat, W. 4C-seq from beginning to end: A detailed protocol for sample preparation and data analysis. Methods 170, 17-32 (2020).
21. Travers, J. G. et al. Pharmacological and Activated Fibroblast Targeting of Gβγ-GRK2 After Myocardial Ischemia Attenuates Heart Failure Progression. J. Am. Coll. Cardiol. 70, 958-971 (2017).
22. Chen, E. Y. et al. Enrichr: interactive and collaborative HTML5 gene list enrichment analysis tool. BMC Bioinformatics 14, 128 (2013).
23. Rockey, D. C., Bell, P. D. & Hill, J. A. Fibrosis—A Common Pathway to Organ Injury and Failure. N. Engl. J. Med. 373, 96 (2015).
24. Jun, J.-I. & Lau, L. F. Resolution of organ fibrosis. J. Clin. Invest. 128, 97-107 (2018).
25. Anand, P. et al. BET bromodomains mediate transcriptional pause release in heart failure. Cell 154: 569-582 (2013).
26. Spiltoir, J. I. et al. BET acetyl-lysine binding proteins control pathological cardiac hypertrophy. J. Mol. Cell Cardiol. 63, 175-179 (2013).
27. Duan, Q. et al. BET bromodomain inhibition suppresses innate inflammatory and profibrotic transcriptional networks in heart failure. Sci. Transl. Med. 9 (2017).
28. Stratton, M. S. et al. Dynamic chromatin targeting of BRD4 stimulates cardiac fibroblast activation. Circ. Res. 125, 662-677 (2019).
29. Antolic, A. et al. BET bromodomain proteins regulate transcriptional reprogramming in genetic dilated cardiomyopathy. BioRxiv (2020).
30. Stratton, M. S. et al. Signal-Dependent Recruitment of BRD4 to Cardiomyocyte Super-Enhancers Is Suppressed by a MicroRNA. Cell Rep. 16, 1366-1378 (2016).
31. Virani, S. S. et al. Heart Disease and Stroke Statistics-2020 Update: A Report from the American Heart Association. Circulation 141, el39-e596 (2020).
32. Shi, J. & Vakoc, C. R. The mechanisms behind the therapeutic activity of BET bromodomain inhibition. Mol. CeI154, 728-736 (2014).
33. Filippakopoulos, P. et al. Selective inhibition of BET bromodomains. Nature 468, 1067-1073 (2010).
34. Nicodeme, E. et al. Suppression of inflammation by a synthetic histone mimic. Nature 468, 1119-1123 (2010).
35. Taniguchi, Y. The Bromodomain and Extra-Terminal Domain (BET) Family: Functional Anatomy of BET Paralogous Proteins. Int. J. Mol. Sci. 17, (2016).
36. Albrecht, B. K. et al. Identification of a Benzoisoxazoloazepine Inhibitor (CPI-0610) of the Bromodomain and Extra-Terminal (BET) Family as a Candidate for Human Clinical Trials. J. Med. Chem. 59, 1330-1339 (2016).
37. Loven, J. et al. Selective inhibition of tumor oncogenes by disruption of super-enhancers. Cell 153, 320-334 (2013).
38. Brown, J. D. et al. BET bromodomain proteins regulate enhancer function during adipogenesis. Proc. Natl. Acad. Sci. USA 115, 2144-2149 (2018).
39. Prabhu, S. D. & Frangogiannis, N. G. The biological basis for cardiac repair after myocardial infarction: from inflammation to fibrosis. Circ. Res. 119, 91-112 (2016).
40. Fu, X. et al. Specialized fibroblast differentiated states underlie scar formation in the infarcted mouse heart. J. Clin. Invest. 128, 2127-2143 (2018).
41. Kanisicak, O. et al. Genetic lineage tracing defines myofibroblast origin and function in the injured heart. Nat. Commun. 7, 12260 (2016).
42. Buenrostro, J. D., Wu, B., Chang, H. Y. & Greenleaf, W. J. ATAC-seq: A Method for Assaying Chromatin Accessibility Genome-Wide. Curr. Protoc. Mol. Biol. 109, 21.29. 1-21.29.9 (2015).
43. Przytycki, P. F. & Pollard, K. S. Semi-supervised identification of cell populations in single-cell ATAC-seq. BioRxiv (2019).
44. Lu, D. et al. Meox1 accelerates myocardial hypertrophic decompensation through Gata 4. Cardiovasc. Res. 114, 300-311 (2018).
45. Li, W., Notani, D. & Rosenfeld, M. G. Enhancers as non-coding RNA transcription units: recent insights and future perspectives. Nat. Rev. Genet. 17, 207-223 (2016).
46. Mahat, D. B. et al. Base-pair-resolution genome-wide mapping of active RNA polymerases using precision nuclear run-on (PRO-seq). Nat. Protoc. 11, 1455-1476 (2016).
47. Dobaczewski, M., Chen, W. & Frangogiannis, N. G. Transforming growth factor (TGF)-β signaling in cardiac remodeling. J. Mol. Cell Cardiol. 51, 600-606 (2011).
48. Gilbert, L. A. et al. CRISPR-mediated modular RNA-guided regulation of transcription in eukaryotes. Cell 154, 442-451 (2013).
49. Whyte, W. A. et al. Master transcription factors and mediator establish super-enhancers at key cell identity genes. Cell 153, 307-319 (2013).
50. Parker, S. C. J. et al. Chromatin stretch enhancer states drive cell-specific gene regulation and harbor human disease risk variants. Proc. Natl. Acad. Sci. USA110, 17921-17926 (2013).
51. Litvinukova, M. et al. Cells and gene expression programs in the adult human heart. BioRxiv (2020).
52. Sivakumar, P. et al. RNA sequencing of transplant-stage idiopathic pulmonary fibrosis lung reveals unique pathway regulation. ERJ Open Research 5 (2019).
53. Yang, J. et al. Targeting LOXL2 for cardiac interstitial fibrosis and heart failure treatment. Nat. Commun. 7, 13710 (2016).
54. Li, D., Wu, J., Bai, Y., Zhao, X. & Liu, L. Isolation and culture of adult mouse cardiomyocytes for cell signaling and in vitro cardiac hypertrophy. J. Vis. Exp. (2014).
55. Love, M. I., Huber, W. & Anders, S. Moderated estimation of fold change and dispersion for RNA-seq data with DESeq2. Genome Biol. 15,550 (2014).
56. Becht, E. et al. Dimensionality reduction for visualizing single-cell data using UMAP. Nat. Biotechnol. (2018).
57. Hu, K. H. et al. ZipSeq: Barcoding for Real-time Mapping of Single Cell Transcriptomes. BioRxiv (2020).
58. Stone, N. R. et al. Context-Specific Transcription Factor Functions Regulate Epigenomic and Transcriptional Dynamics during Cardiac Reprogramming. Cell Stem Cell 25, 87-102.e9 (2019).
59. Acharya, A., Baek, S. T., Banfi, S., Eskiocak, B. & Tallquist, M. D. Efficient inducible Cre-mediated recombination in Tcf21 cell lineages in the heart and kidney. Genesis 49, 870-877 (2011).
60. Kwak, H., Fuda, N. J., Core, L. J. & Lis, J. T. Precise maps of RNA polymerase reveal how promoters direct initiation and pausing. Science 339, 950-953 (2013).
61. Langmead, B. & Salzberg, S. L. Fast gapped-read alignment with Bowtie 2.

Nat. Methods 9, 357-359 (2012).
62. Guo, Y., Mahony, S. & Gifford, D. K. High resolution genome wide binding event finding and motif discovery reveals transcription factor spatial binding constraints. PLoS Comput. Biol. 8, e1002638 (2012).
63. Liao, Y., Smyth, G. K. & Shi, W. featureCounts: an efficient general purpose program for assigning sequence reads to genomic features. Bioinformatics 30, 923-930 (2014).
64. Quinlan, A. R. & Hall, 1. M. BEDTools: a flexible suite of utilities for comparing genomic features. Bioinformatics 26, 841-842 (2010).
65. Stadhouders, R. et al. Multiplexed chromosome conformation capture sequencing for rapid genome-scale high-resolution detection of long-range chromatin interactions. Nat. Protoc. 8, 509-524 (2013).
66. Krijger, P. H. L., Geeven, G., Bianchi, V., Hilvering, C. R. E. & de Laat, W. 4C-seq from beginning to end. A detailed protocol for sample preparation and data analysis. Methods 170, 17-32 (2020).
67. Travers, J. G. et al. Pharmacological and Activated Fibroblast Targeting of Gβγ-GRK2 After Myocardial Ischemia Attenuates Heart Failure Progression. J. Am.

Coll. Cardiol. 70, 958-971 (2017).
68. Chen, E. Y. et al. Enrichr: interactive and collaborative HTML5 gene list enrichment analysis tool. BMC Bioinformatics 14, 128 (2013).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements:

1. A method comprising contacting at least one test agent with a population of cells to provide a test assay mixture and measuring Meox1 levels to thereby identify one or more Meox1 modulating agents.
2. The method of statement 1, wherein the population of cells comprises fibroblasts, resting fibroblasts, myofibroblasts or a combination thereof.
3. The method of statement 1 or 2, wherein the population of cells comprises activated fibroblasts.
4. The method of any of statements 1-3, wherein the fibroblasts are activated by TGFβ
5. The method of any of statements 1-4, wherein measuring Meox1 levels comprises measuring chromatin accessibility of a Meox1 regulatory element.
6. The method of statement 5, wherein the Meox1 regulatory element is an enhancer.
7. The method of statement 5 or 6, wherein the Meox1 regulatory element is a peak 9/10 enhancer.
8. The method of statement 5, 6 or 7, wherein the Meox1 regulatory element is on human chromosome 17 between about positions 43,589,381 and 43,595,263.
9. The method of any of statements 1-8, wherein the population of cells comprises fibroblasts from cardiac tissues, lung tissues, liver tissues, kidney tissues, or a combination thereof.
10. The method of any of statements 1-9, wherein measuring Meox1 levels comprises measuring Meox1 transcript or protein levels or wherein measuring Meox1 levels comprises measuring absolute numbers of observed Meox1 transcripts (UMI counts) per gene per cell.
11. The method of any of statements 1-10, wherein one or more of the Meox1 modulating agents increase Meox1 levels.
12. The method of any of statements 1-10, wherein one or more of the Meox1 modulating agents reduce Meox1 levels.
13. The method of any of statements 1-12, wherein one or more of the Meox1 modulating agents reduce Meox1 enhancer activity.
14. The method of any of statements 1-13, wherein one or more of the Meox1 modulating agents reduce chromosomal accessibility of a Meox1 enhancer.
15. The method of statement 14, wherein the Meox1 enhancer is on human chromosome 17 between about positions 43,589,381 and 43,595,263.
16. The method of any of statements 1-15, further comprising administering one or more of the Meox1 modulating agents to an animal model of a heart condition or disease and determining whether one or more of the Meox1 modulating agents reduces the symptoms or severity of the heart condition or disease to thereby identity a therapeutic agent.
17. The method of statement 16, further comprising administering one or more of the therapeutic agents to a patient.
18. The method of statement 17, wherein the patient is need thereof of the one or more of the therapeutic agents.
19. The method of statement 16, 17, or 18, wherein the animal model or the patient has cardiac fibrosis, lung fibrosis, kidney fibrosis, liver fibrosis, heart failure, congestive heart failure, myocardial infarction, cardiac ischemia, myocarditis, arrhythmia cardiomyopathy, dilated cardiomyopathy, coronary artery disease, hypertension, valvular heart disease, hypertrophic cardiomyopathy (HCM), familial dilated cardiomyopathy (FDCM), restrictive cardiomyopathy (RCM), arrhythmogenic cardiomyopathy (AVC), unclassified cardiomyopathy, or a combination thereof.
20. The method of any of statements 1-19, wherein the population of cells are from a patient or subject seeking treatment for a heart condition or disease.
21. The method of statement 20, wherein the patient or subject exhibits increased Meox1 levels in cardiac fibroblasts, increased Meox1 nascent transcription, increased chromatin accessibility in a Meox1 regulatory element within cardiac fibroblasts, or a combination thereof.
22. The method of statement 20 or 21, comprising knockout or knockdown of the Meox1 regulatory element within the patient's or subject's fibroblasts, myofibroblasts or a combination thereof.
23. The method of statement 20, 21, or 22, comprising in vivo knockout or knockdown of the Meox1 regulatory element within the patient's or subject's fibroblasts.
24. The method of statement 23, wherein knockout or knockdown of the Meox1 regulatory element comprises CRISPR modification of the Meox1 regulatory element, contacting a Meox1 inhibitory nucleic acid with the Meox1 regulatory element, or a combination thereof.
25. The method of statement 24, wherein the Meox1 inhibitory nucleic acid is an antisense oligonucleotide, a small interfering RNA (siRNA), a small hairpin RNA (shRNA), a CRISPR guide RNA, a CRISPR ribonucleoprotein comprising a guide RNA and a cas nuclease, or a combination thereof.
26. The method of statement 22-24 or 25, comprising in vitro knockout or knockdown of the Meox1 regulatory element within a population of the patient's fibroblasts to generate modified fibroblasts and reintroducing the modified fibroblasts to the patient.
27. The method of any of statements 20-25 wherein the fibroblasts are cardiac fibroblasts, myofibroblasts, or a combination thereof.
28. A method comprising administering a therapeutic agent to a subject comprising fibroblasts exhibiting increased chromatin accessibility in a Meox1 regulatory element, increased Meox1 expression, increased Meox1 nascent transcript levels, or a combination thereof.
29. The method of statement 28, wherein the Meox1 regulatory element is an enhancer.
30. The method of statement 28 or 29, wherein the Meox1 regulatory element is a peak 9/10 enhancer.
31. The method of any of statement 28-30, wherein the Meox1 regulatory element is on human chromosome 17 between about positions 43,589,381 and 43,595,263.
32. The method of any of statements 28-31, wherein the therapeutic agent is a guide RNA, a ribonucleoprotein complex comprising a cas nuclease, an inhibitory nucleic acid, a chromatin stabilizing agent, or a combination thereof.
33. A method comprising administering to a subject an agent that modulates Meox1 transcription, Meox1 translation, or MEOX1 protein function, to thereby treat a cardiac disease or condition.
34. The method of statement 33, wherein the agent inhibits a combination of one or more of Meox1 transcription, Meox1 translation, or MEOX1 protein function.
35. The method of statement 33 or 34, wherein the agent inhibits Meox1 transcription, Meox1 translation, or MEOX1 protein function.
36. The method of statement 33, 34, or 35, wherein the agent directly or indirectly modulates an enhancer operably linked to a Meox1 gene.
37. The method of statement 33-35 or 36, wherein the agent binds to an enhancer operably linked to a Meox1 gene or MEOX1 coding region.
38. The method of statement 33-36 or 37, wherein the agent is an RNA interference (RNAi) nucleic acid that reduces Meox1 translation.
39. The method of statement 33-37 or 38, wherein the Meox1 inhibitory nucleic acid is an antisense oligonucleotide, a small interfering RNA (siRNA), a small hairpin RNA (shRNA), or a combination thereof.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed:

1. A method comprising contacting at least one test agent with a population of cells to provide a test assay mixture and measuring Meox1 levels to thereby identify one or more Meox1 modulating agents.

2. The method of claim 1, wherein the population of cells comprises fibroblasts, activated fibroblasts, resting fibroblasts, myofibroblasts, activated myofibroblasts, or a combination thereof.

3. The method of claim 1, wherein measuring Meox1 levels comprises measuring chromatin accessibility of a Meox1 enhancer, measuring Meox1 transcript levels, measuring nascent Meox1 transcript levels, or a combination thereof.

4. The method of claim 3, wherein the Meox1 enhancer is on human chromosome 17 between about positions 43,589,381 and 43,595,263.

5. The method of claim 1, wherein measuring Meox1 levels comprises measuring absolute numbers of observed Meox1 transcripts or Meox1 nascent transcripts per gene per cell.

6. The method of claim 1, wherein at least one of the test agents is an antisense oligonucleotide, a small interfering RNA (siRNA), a small hairpin RNA (shRNA), a CRISPR guide RNA, a CRISPR ribonucleoprotein comprising a guide RNA and a cas nuclease, or a combination thereof.

7. The method of claim 1, wherein one or more of the Meox1 modulating agents reduces Meox1 levels, reduces Meox1 enhancer activity, or a combination thereof.

8. The method of claim 1, wherein one or more of the Meox1 modulating agents reduces chromatin accessibility of a Meox1 enhancer, reduces Meox1 transcript levels, reduces nascent Meox1 transcript levels, or a combination thereof.

9. The method of claim 1, further comprising administering one or more of the Meox1 modulating agents to an animal model of a heart condition or disease and determining whether one or more of the Meox1 modulating agents reduces the symptoms or severity of the heart condition or disease to thereby identity a therapeutic agent.

10. The method of claim 9, further comprising administering one or more of the test agents or therapeutic agents to a subject.

11. The method of claim 10, wherein the subject has or is suspected of having cardiac fibrosis, lung fibrosis, kidney fibrosis, liver fibrosis, heart failure, congestive heart failure, myocardial infarction, cardiac ischemia, myocarditis, arrhythmia cardiomyopathy, dilated cardiomyopathy, coronary artery disease, hypertension, valvular heart disease, hypertrophic cardiomyopathy (HCM), familial dilated cardiomyopathy (FDCM), restrictive cardiomyopathy (RCM), arrhythmogenic cardiomyopathy (AVC), unclassified cardiomyopathy, or a combination thereof.

12. The method of claim 1, wherein the population of cells is from a patient seeking treatment for a condition or disease.

13. The method of claim 12, wherein the patient having the population of cells exhibits increased Meox1 levels in fibroblasts, increased nascent Meox1 levels in fibroblasts, increased chromatin accessibility in a Meox1 enhancer, within fibroblasts, or a combination thereof.

14. The method of claim 13, wherein the fibroblasts are cardiac fibroblasts, lung fibroblasts, liver fibroblasts, kidney fibroblasts, or a combination thereof.

15. A method comprising contacting cells with an agent that inhibits Meox1 RNA transcription, Meox1 chromatin accessibility, Meox1 RNA processing, or Meox1 translation.

16. The method of claim 15, wherein the cells comprise fibroblasts, myofibroblasts, activated fibroblasts, activated myofibroblasts, or a combination thereof.

17. The method of claim 16, wherein the fibroblasts are cardiac fibroblasts, lung fibroblasts, liver fibroblasts, kidney fibroblasts, or a combination thereof.

18. The method of claim 15, wherein the agent knocks down or knocks out Meox1 transcription, knocks down or knocks out Meox1 enhancer activity, or a combination thereof.

19. The method of claim 15, wherein the agent comprises one or more inhibitory nucleic acids, guide RNAs, cas nucleases, cas nuclease: guide RNA ribonucleoprotein complexes, or combinations thereof.

20. The method of claim 15, wherein contacting the cells occurs in vitro.

21. The method of claim 20, which further comprises isolating modified cells and administering them to a subject with a cardiac condition or cardiac disease.

22. The method of claim 20 wherein the cells contacted in vitro were from the subject later administered the modified cells.

23. The method of claim 15, wherein contacting cells occurs in vivo by administering the agent to a subject.

24. The method of claim 23, wherein the subject has or is suspected of having cardiac fibrosis, lung fibrosis, kidney fibrosis, liver fibrosis, heart failure, congestive heart failure, myocardial infarction, cardiac ischemia, myocarditis, arrhythmia cardiomyopathy, dilated cardiomyopathy, cardiac artery disease, hypertension, valvular heart disease, hypertrophic cardiomyopathy (HCM), familial dilated cardiomyopathy (FDCM), restrictive cardiomyopathy (RCM), arrhythmogenic cardiomyopathy (AVC), unclassified cardiomyopathy, or a combination thereof.
